(19) United States
(12) Patent Application Publication
(10) Pub. No.: US 2010/0323967 A1
(43) Pub. Date: Dec. 23, 2010

(54) RNA Antagonist Compounds for the Modulation of MCL-1

(75) Inventors: Jens Bo Rode Hansen, Charlottenlund (DK); Anja Mølhart Høg, Virum (DK)

Correspondence Address: STERNE, KESSLER, GOLDSTEIN & FOX P.L.L.C., 1100 NEW YORK AVENUE, N.W., WASHINGTON, DC, 20005, US

(73) Assignee: Santaris Pharma A/S, Horsholm (DK)

(21) Appl. No.: 12/746,745

(22) PCT Filed: Dec. 5, 2008
(86) PCT No.: PCT/EP08/66920

§ 371 Date: Sep. 7, 2010

Related U.S. Application Data
(60) Provisional application No. 61/012,191, filed on Dec. 7, 2007.
(60) Provisional application No. 61/095,955, filed on Sep. 11, 2008.

(51) Int. Cl. A61K 38/14; C07H 21/00; C07H 21/04; A61K 31/7088; C12N 5/071; A61K 31/711; C07K 14/00; A61P 35/00; A61P 37/00

(52) U.S. Cl. 514/16.6; 536/23.1; 514/44 A; 435/375; 530/395; 514/19.3; 514/20.9

(57) ABSTRACT

Oligonucleotides directed against the Mcl-1 gene are developed for modulating the expression of Mcl-1 protein. The compositions comprise oligonucleotides, particularly antisense oligonucleotides, targeted to nucleic acids encoding Mcl-1. Methods of using these compounds for modulation of Mcl-1 expression and for the treatment of diseases associated with over expression of Mcl-1 are provided. Examples of such diseases include cancer and systemic mastocytosis.

### RELATED CASES

This application claims the benefit under 35 U.S.C. §119(e) of U.S. Provisional Application Ser. No. 61/012,191, filed 7 Dec. 2007, and U.S. 61/095,955, filed 11 Sep. 2008, the disclosures of which are both incorporated herein by reference in their entirety.

### FIELD OF THE INVENTION

The present invention provides compounds, conjugates, compositions and methods for modulating the expression of Mcl-1. In particular, this invention relates to oligomeric compounds (oligomers), which target the Mcl-1 mRNA in a cell, leading to reduced expression of Mcl-1. Reduction of Mcl-1 expression is beneficial for a range of medical disorders, such as cell proliferation disorders, such as cancer or mastocytosis.

### BACKGROUND

The role of Mcl-1 in regulating cell fate has made it a target of interest in many studies of apoptosis and hyperproliferative diseases. Many reports have demonstrated the importance of inhibiting Mcl-1 expression to increase apoptosis and regulate neoplastic disease. Mcl1 mRNA exists in two forms generated by alternative splicing of the pre-mRNA, a longer gene product (isoform 1) enhances cell survival by inhibiting apoptosis, whilst a alternatively spliced shorter gene product promotes apoptosis.

Several studies have reported use of Mcl-1 antisense oligonucleotides or siRNA to inhibit Mcl-1 expression, increase apoptosis, decrease cell viability and/or decrease tumor weight of normal cells, cancer cell lines or xenograft tumors. Henson et al. ((2006) Clin. Cancer Res. 12(3):845-853) disclose a Mcl-1 antisense oligonucleotide which sensitizes breast cancer cell lines to apoptosis. Selzer et al. ((2002) Mol. Med. S(72:877-884) disclose inhibition of Mcl-1 expression in melanoma cells using an antisense oligonucleotide targeting Mcl-1 and Skvara et al. ((2005) Anticancer Res. 25(4):2697-2703) show a Mcl-1 antisense oligonucleotide which sensitizes human melanoma cells to ionizing radiation-induced apoptosis. Sieghart et al. ((2006) J. Hepatol. 44(1):\5\-\51) discuss the finding the Mcl-1 is overexpressed in hepatocellular carcinoma cells lines and disclose a Mcl-1 antisense oligonucleotide which decreases protein expression, increases apoptosis, decreases cell survival and sensitizes HCC cells to chemotherapy. Similarly, Song et al. ((2005) Cancer Biol. Ther. 4 (3)-0.267-276) show that Mcl-1 is overexpressed in human lung cancer cells and treatment with a Mcl-1 antisense oligonucleotide resulted in an increase in apoptosis. Mcl-1 inhibition also caused sensitization of the lung cancer cells to apoptosis induced by chemotherapeutic agents and radiation.

Aichberger et al. ((2005) Blood J05(8J:3303-30U) discuss a Mcl-1 antisense oligonucleotide and siRNA which inhibit expression of Mcl-1 in chronic myeloid leukemia cells and decreased cell viability. Mcl-1 antisense oligonucleotide synergized with the BCR/ABL inhibitor Imatinib to produce growth arrest. Derenne et al. ((2002) Blood 100(1):\94-\99) and Zhang et al. ((2002) Blood 99(6):1 and 85-IS93) discuss the use of Mcl-1 antisense oligonucleotide to decrease expression of Mcl-15 decrease cell viability and increase apoptosis of multiple myeloma cells lines and primary cells.

Mcl-1 has also been shown to exhibit increased expression in a variety of hematopoietic cells lines, including B cells, monocytes, macrophages and polymorphonuclear cells, and treatment of these cells with Mcl-1 antisense oligonucleotide reduces target expression, and increases apoptosis (Michels et al. (2004) Oncogene 23(28):4 and 18-4827; Sly et al. (2003) J. Immunol. 170(1):430-437; Liu et al. (2001) J. Exp. Med. 194(2):\U-\26; Moulding et al. (2000) Blood 96(5)\\156-17 '3; and Leuenroth e/tf/. (2000) J. Leukoc. Biol 68:158-166).

Thallinger et al. ((2004) CHn. Cancer Res. 70:4185-4191) disclose an antisense oligonucleotide targeted to Mcl-1 which decreases expression of Mcl-1 in sarcoma xenotransplants and when used in combination with cyclophosphamide, reduces tumor weight and increases tumor cell apoptosis. Thallinger et al. ((2003) J. Invest. Dermatol. 120(6):1081-1086) show a Mcl-1 antisense oligonucleotide administered systemically with or without dacarbazine in a human melanoma SCID mouse xenotransplantation model decreased target protein expression, increased apoptosis and decreased tumor weight.

U.S. Pat. No. 5,470,955 reports on antibodies which specifically bind the Mcl-1 polypeptide and their use to treat cell proliferative disorders associated with Mcl-1, such as myeloid cell leukaemia. U.S. Pat. No. 5,470,955 also refers to antisense polynucleotides, complementary to the Mcl-1 sequence, which are capable of inhibiting production of the Mcl-1 polypeptide including antisense oligomers of about 15 nucleotides.

U.S. Pat. No. 6,001,992 reports on 20 antisense gapmer phosphorothioate oligonucleotides targeting Mcl-1 consisting of 5 nucleobase 2′-MOE wings, and a central region of 10 DNA nucleotides. The efficacy of the oligomers for down-regulation of Mcl-1 in cell culture ranged between 2.6% to 62.5% with 100 nM dosage. Five of the oligonucleotides were found to down-regulate Mcl-1 mRNA in the treated cells by at least 50%.

WO 2007/109174 also reports on antisense gapmer phosphorothioate oligonucleotides targeting Mcl-1 consisting of 5 nucleobase 2′-MOE wings, and a central region of 10 DNA nucleotides, some of which are substantially overlapping with those disclosed in U.S. Pat. No. 6,001,992. Although the experimental systems appear somewhat different, analysis of about 80 oligonucleotides identified 12 oligonucleotides which inhibited Mcl-1 mRNA by at least 70%, although several of these overlap with oligonucleotides which were comparatively ineffective.

WO 2007/147613 reports on methods for the treatment of myelodysplastic syndromes, such as systemic mastocytosis, lymphomas and leukemias and solid tumors with a pharmaceutical combination of a FLT-3 kinase inhibitor and an antisense oligonucleotide or Mcl1 specific RNAi construct.

In an exemplary aspect, the present invention provides improved oligonucleotides, particularly oligonucleotides based on locked nucleic acid chemistry, which target the Mcl-1 mRNA.

### SUMMARY OF THE INVENTION

The invention provides an oligomer of between 10-50, such as 10-30 nucleotides, in length, which comprises a contiguous nucleotide sequence of a total of between 10-30 nucleotides, wherein said contiguous nucleotide sequence is at least 80% (e.g., 85%, 90%, 95%, 98%, or 99%) homologous to a region corresponding to the reverse complement of a mammalian Mcl1 gene or mRNA, or naturally occurring variant thereof.

The invention provides an oligomer of between 10-50, such as 10-30 nucleotides, in length, which comprises a contiguous nucleotide sequence of a total of between 10-30 nucleotides, wherein said contiguous nucleotide sequence is at least 80% (e.g., 85%, 90%, 95%, 98%, or 99%) homologous to a nucleic acid sequence selected from the group consisting of SEQ ID NO 2-18, SEQ ID NO 77-82 and SEQ ID NO 95-117.

The invention provides for a conjugate comprising the oligomer according to the invention, and at least one non-nucleotide or non-polynucleotide moiety covalently attached to said oligomer.

The invention provides for a pharmaceutical composition comprising the oligomer or the conjugate according to the invention, and a pharmaceutically acceptable diluent, carrier, salt or adjuvant.

The oligomer or conjugate of the invention may be a medicament. The invention provides for the oligomer or the conjugate according to invention, for use as a medicament, such as for the treatment of cell proliferation disorders, such as cancer or mastocytosis.

The invention provides for the use of an oligomer or the conjugate according to the invention, for the manufacture of a medicament for the treatment of cell proliferation disorders, such as cancer or mastocytosis.

The invention provides for a method of treating cell proliferation disorders, such as cancer or mastocytosis, said method comprising administering an (e.g. an effective amount of) an oligomer, a conjugate or a pharmaceutical composition according to the invention, to a patient suffering from, or likely to suffer from cell proliferation disorders, such as cancer or mastocytosis.

The invention provides for a method for the inhibition of Mcl1 in a cell which is expressing Mcl1, said method comprising administering an (e.g. an effective amount of) oligomer, or a conjugate according to the invention to said cell so as to effect the inhibition of Mcl1 in said cell.

In some exemplary embodiments, the oligomer comprises at least one LNA unit. Suitably the at least one LNA unit is within the contiguous nucleotides sequence.

The invention provides for a pharmaceutical composition comprising the oligomer or conjugate according to the invention, a further active ingredient and a pharmaceutically acceptable diluent, carrier, salt or adjuvant.

The invention further provides for an oligomer or conjugate according to the invention, for use in medicine.

The invention provides for a medicament comprising the oligomer or conjugate of the invention, and optionally a further active ingredient. The medicament suitably comprises a pharmaceutically acceptable diluent, carrier, salt or adjuvant.

The invention further provides for the use of the oligomer of the invention for the manufacture of a medicament for the treatment of one or more of the diseases referred to herein, such as cell hyperproliferation disorders, such as cancer or mastocytosis.

The invention further provides for an oligomer according to the invention, for use for the treatment of one or more of the diseases referred to herein, such as cell hyperproliferation disorders, such as cancer or mastocytosis. The invention further provides for an oligomer according to the invention, for use for the treatment of one or more of the diseases referred to herein.

Pharmaceutical and other compositions comprising the oligomer of the invention are also provided. Further provided are methods of down-regulating the expression of Mcl-1 in cells or tissues comprising contacting said cells or tissues, in vitro or in vivo, with one or more of the oligomers, conjugates or compositions of the invention.

Also disclosed are methods of treating an animal or a human, suspected of having or being prone to a disease or condition, associated with expression, or over-expression, of Mcl-1, by administering to said animal or human a therapeutically or prophylactically effective amount of one or more of the oligomers, conjugates or compositions of the invention. Further, methods of using oligomers for the inhibition of expression of Mcl-1, and for treatment of diseases associated with activity of Mcl-1 are provided.

The invention provides for a method for treating a cell hyperproliferation disease such as cancer and inflammatory diseases, such as rheumatoid arthritis, in a patient, said method comprising administering an oligomer, a conjugate, or a pharmaceutical composition according to the invention to a patient in need thereof.

The invention provides for a method for treating a cell hyperproliferation disease such as myelodysplastic syndromes, such as systemic mastocytosis, lymphomas and leukemias and solid tumors, in a patient, said method comprising administering an oligomer, a conjugate, or a pharmaceutical composition according to the invention to a patient in need thereof.

The invention provides for a method for treating an inflammatory disease, such as rheumatoid arthritis, in a patient, said method comprising administering an oligomer, a conjugate, or a pharmaceutical composition according to the invention to a patient in need thereof.

The invention provides for a method of inhibiting or reducing the expression of Mcl-1 in a cell or a tissue, the method comprising the step of contacting said cell or tissue with on oligomer, a conjugate, or a pharmaceutical composition according to the invention so that expression of Mcl-1 is inhibited or reduced.

The invention provides for a method of triggering apoptosis in a cell, such as a cancer cell, said method comprising the step of contacting said cell or tissue with on oligomer, a conjugate, or a pharmaceutical composition according to the invention so that either expression of Mcl-1 is inhibited or reduced and/or apoptosis is triggered.

### BRIEF DESCRIPTION OF THE FIGURES

FIG. 1. Oligonucleotides presented in Table 3 were evaluated for their potential to knockdown Mcl-1 mRNA at concentrations of 1, 5 and 25 nM in 15PC3 cells 24 hours after transfection using quantitative real-time PCR. All results were normalised to GAPDH or beta-Actin and inhibition of Mcl-1 mRNA is shown as percent of untreated control (mock).

FIG. 2: SEQ ID NO: 36, 42, 47, 50, 55, 58, 61, 64, and 66 were evaluated for their potential to induce apoptosis in 15PC3 cells 24 and 48 hours after transfection at concentrations of 1, 5, and 25 nM measured as caspase 3/7 activity. All results were normalised to untreated control (mock) and is given as fold induction relative to untreated control.

FIG. 3. SEQ ID NO: 36, 42, 47, 50, 55, 58, 61, 64, and 66 were evaluated for their potential to induce apoptosis in HUH-7 cells 24, 48 and 72 hours after transfection at concentrations of 1, 5, and 25 nM measured as caspase 3/7 activity. All results were normalised to untreated control (mock) and is given as fold induction relative to untreated control.

FIG. 4. Location of preferred target regions of the human Mcl-1 mRNA targeted by oligomers according to the invention. Although 16mer target sites have been shown, suitably these target regions may comprise an additional 4 bases 5′ or 3′ to the regions shown—i.e. regions of up to 24 contiguous nucleotides.

FIG. 5. SEQ ID NO 70: Homo sapiens myeloid cell leukemia sequence 1 (BCL2-related) (MCL1), transcript variant 1, mRNA. Accession number NM_{—}021960

FIG. 6. SEQ ID NO 1: Homo sapiens myeloid cell leukemia sequence 1 (BCL2-related) (MCL1), transcript variant 2, mRNA, Accession number NM_{—}182763.

FIG. 7. SEQ ID NO 71: Homo sapiens myeloid cell leukemia sequence 1 (BCL2-related) (MCL1), transcript variant 1, protein.

FIG. 8. SEQ ID NO 72: Homo sapiens myeloid cell leukemia sequence 1 (BCL2-related) (MCL1), transcript variant 2, protein.

FIG. 9. SEQ ID NO 73: Mouse Mcl-1 mRNA sequence.

FIG. 10. SEQ ID NO 74: Mouse Mcl-1 protein sequence.

FIG. 11. SEQ ID NO 75: Rhesus monkey Mcl-1 mRNA sequence.

FIG. 12. SEQ ID NO 76: Rhesus monkey Mcl-1 protein sequence.

FIG. 13. Alignment between the two alternative splice variants of the Mcl-1 mRNA (SEQ ID NO 1 and SEQ ID NO 70). SEQ ID NO 1 is the mRNA sequence of the short, proapoptotic isoform of Mcl-1, SEQ ID NO:70 is the mRNA sequence of the long, antiapoptotic isoform of Mcl-1.

FIG. 14. Down regulation of Mcl1 mRNA normalised to GAPDH from unassisted uptake of anti Mcl1 oligomers (5 μM) in 15PC3 cells.

FIG. 15. SEQ ID NO: 50 and SEQ ID NO: 64 were evaluated for their potential to down-regulate Mcl-1 in vivo in mouse liver tissue. Animals were dosed daily with 5 mg/kg of antisense oligonucleotide or saline for 2 weeks. Liver tissue was harvested for RNA analysis 24 hours after the last dosing.

FIG. 16. SEQ ID NO: 90, SEQ ID NO: 91 and SEQ ID NO: 92 were evaluated for their potential to down-regulate Mcl-1 in vivo in mouse liver tissue. Animals were dosed thrice weekly with 10 mg/kg of antisense oligonucleotide or saline for a total of 7 doses. Liver tissue was harvested for RNA analysis 48 hours after the last dosing.

FIG. 17. Selected oligonucleotides presented in Table 5 were evaluated for their potential to down-regulate Mcl-1 mRNAs using natural uptake at 5 μM in 15PC3 cells.

FIG. 18. Selected oligonucleotides presented in Table 5 were evaluated for their potential to down-regulate Mcl-1 mRNAs after 96 hours incubation at 5 μM in Namalwa cells.

FIG. 19. Selected oligonucleotides presented in Table 5 were evaluated for their potential to induce Caspase 3/7 activity in Namalwa cells after 0, 24, 48, and 96 hours incubation at 5 μM.

FIG. 20. The cell viability of Namalwa cells after 0, 24, 48, and 96 hours incubation with selected oligonucleotides presented in Table 5 at 5 μM were measured via MTS assay.

FIG. 21. Selected oligonucleotides presented in Table 5 were evaluated for their potential to down-regulate Mcl-1 mRNAs after 96 hours incubation at 5 μM in K562 cells.

FIG. 22. Selected oligonucleotides presented in Table 5 were evaluated for their potential to induce Caspase 3/7 activity in K562 cells after 0, 24, 48, and 96 hours incubation at 5 μM.

FIG. 23. The cell viability of K562 cells after 0, 24, 48, and 96 hours incubation with selected oligonucleotides presented in Table 5 at 5 μM were measured via MTS assay.

FIG. 24. Selected oligonucleotides presented in Table 5 were evaluated for their potential to induce Caspase 3/7 activity in myeloid leukemia K562 cells after 72 hours incubation at 5 μM and after treatment with the histone deacetylase inhibitor Trichostatin A at indicated concentrations for 24 hours.

FIG. 25. Selected oligonucleotides presented in Table 5 were evaluated for their potential to induce Caspase 3/7 activity in myeloid leukemia K562 cells after 72 hours incubation at 5 μM and after treatment with the alkylating agent Cisplatin at indicated concentrations for 24 hours.

FIG. 26. The cell viability of Burkitt's lymphoma Namalwa cells after 96 hours incubation with selected oligonucleotides presented in Table 5 at 5 μM and after treatment with the glucocorticoid dexamethsaone for 48 hours. Cell viability was measured via MTS assay.

FIG. 27. Selected oligonucleotides presented in Table 5 were evaluated for their potential to induce Caspase 3/7 activity in hepatocellular carcinoma HepG2 cells after 96 hours incubation at 5 μM and after treatment with the alkylating agent Cisplatin at indicated concentrations for 48 hours.

FIG. 28: Cell viability when combining Mcl-1 targeting oligos SEQ ID NOs: 64 and 91 with scrambled control oligo or Bcl-2 targeting oligo SEQ ID NO: 128 in 15PC3 cell line using natural uptake.

FIG. 29: Caspase 3/7 induction when combining Mcl-1 targeting oligos SEQ ID NOs: 64 and 91 with scrambled control oligo or Bcl-2 targeting oligo SEQ ID NO: 128 in 15PC3 cell line using natural uptake.

### DETAILED DESCRIPTION OF THE INVENTION

### The Oligomer

The present invention employs oligomeric compounds (referred herein as oligomers), for use in modulating the function of nucleic acid molecules encoding mammalian Mcl1, such as the Mcl1 nucleic acids shown in SEQ ID 1 or 70, and naturally occurring variants of such nucleic acid molecules encoding mammalian Mcl1. The term “oligomer” in the context of the present invention, refers to a molecule formed by covalent linkage of two or more nucleotides (i.e. an oligonucleotide). The oligomer consists or comprises of a contiguous nucleotide sequence of between 10-50, such as 10-30 nucleotides in length. The term “oligomer” in the context of the present invention, refers to a molecule formed by covalent linkage of two or more nucleotides (i.e. an oligonucleotide). Herein, each single nucleotide, such as the nucleotides present in the oligomer of the invention, may also be referred to as a “monomer” or “unit”. In some embodiments, the oligomer consists or comprises of a contiguous nucleotide sequence of between 10-30 nucleotides in length (i.e. comprises or consists of from 10-30 covalently linked monomers).

In various embodiments, the compound of the invention does not comprise RNA (units). It is preferred that the compound according to the invention is a linear molecule or is synthesised as a linear molecule. The oligomer is a single stranded molecule, and preferably does not comprise short regions of, for example, at least 3, 4 or 5 contiguous nucleotides, which are complementary to equivalent regions within the same oligomer (i.e. duplexes)—in this regards, the oligomer is not (essentially) double stranded. In some embodiments, the oligomer is essentially not double stranded, such as is not a siRNA. In various embodiments, the oligomer of the invention may consist entirely of the contiguous nucleotide region. Thus, the oligomer is not substantially self complementary. siRNAs comprise of 2 complementary short RNA (or equivalent nucleobase units) sequences, such as between 21 and 23 nts long, with, typically a 2 nt 3′ overhang on either end. In order to enhance in vivo uptake, the siRNAs may be conjugated, such as conjugated to a sterol, such as a cholesterol group (typically at the 3′ or 5′ termini of one or both of the strands).

The invention further provides target sequences in the Mcl1 mRNA or gene, or an allelic variant thereof, in particular those corresponding to a sequence selected from the group consisting of SEQ ID NOS: 2-18, 77-82, and 95-117, wherein antisense oligonucleotides corresponding to said target sequences are capable of down-regulating Mclb 1. A variant sequence may have at least 60%, more preferably at least 70%, more preferably at least 80%, more preferably at least 85%, more preferably at least 90%, more preferably at least 91%, at least 92%, at least 93%, at least 94%, at least 95% sequence homology to a target sequence in Mcl1. Typically, an oligomer of the invention corresponding to said variant sequences is still capable of down-regulating Mcl1.

Specific designs of LNA oligonucleotides are also disclosed, for example those shown in SEQ ID NOS 19-35, 83-88, 36-69, 89-94n and 118-127. The oligomers of the invention are considered to be potent inhibitors of Mcl1 mRNA and protein expression.

### The Target

Suitably the oligomer of the invention is capable of down-regulating expression of the Mcl1 gene. In this regards, the oligomer of the invention can effect the inhibition of Mcl1, typically in a mammalian such as a human cell. In some embodiments, the oligomers of the invention binds to the target nucleic acid and effect inhibition of expression of at least 10% or 20% compared to the normal expression level, more preferably at least a 30%, 40%, 50%, 60%, 70%, 80%, 90% or 95% inhibition compared to the normal expression level. In some embodiments, such modulation is seen when using between 0.04 and 25 nM, such as between 0.8 and 20 nM concentration of the compound of the invention. In the same or a different embodiment, the inhibition of expression is less than 100%, such as less than 98% inhibition, less than 95% inhibition, less than 90% inhibition, less than 80% inhibition, such as less than 70% inhibition. Modulation of expression level may be determined by measuring protein levels, e.g. by the methods such as SDS-PAGE followed by western blotting using suitable antibodies raised against the target protein. Alternatively, modulation of expression levels can be determined by measuring levels of mRNA, e.g. by northern blotting or quantitative RT-PCR. When measuring via mRNA levels, the level of down-regulation when using an appropriate dosage, such as between 0.04 and 25 nM, such as between 0.8 and 20 nM concentration, is, in some embodiments, typically to a level of between 10-20% the normal levels in the absence of the compound of the invention.

The invention therefore provides a method of down-regulating or inhibiting the expression of Mcl1 protein and/or mRNA in a cell which is expressing Mcl1 protein and/or mRNA, said method comprising administering the oligomer or conjugate according to the invention to said cell to down-regulating or inhibiting the expression of Mcl1 protein and/or mRNA in said cell. The administration is typically performed as an effective amount of said oligomer. The cell may, in some embodiments be a cancer cell. The cell is some embodiments may be a mast cell or a CD34+mast cell precursor cell. The cell is some embodiments may be an immune cell such as a B cell. Suitably the cell is a mammalian cell such as a human cell. The administration may occur, in some embodiments, in vitro. The administration may occur, in some embodiments, in vivo.

The term “target nucleic acid”, as used herein refers to the DNA or RNA encoding mammalian Mcl1 polypeptide, such as human Mcl1, such as SEQ ID NO: 1 or 70. Mcl1 encoding nucleic acids or naturally occurring variants thereof, and RNA nucleic acids derived therefrom, preferably mRNA, such as pre-mRNA, although preferably mature mRNA. In some embodiments, for example when used in research or diagnostics the “target nucleic acid” may be a cDNA or a synthetic oligonucleotide derived from the above DNA or RNA nucleic acid targets. The oligomer according to the invention is preferably capable of hybridising to the target nucleic acid. It will be recognised that SEQ ID NO: 1 and 70 are cDNA sequences, and as such, corresponds to mature Mcl1 mRNA target sequences, although uracil is replaced with thymidine in the cDNA sequences.

The term “naturally occurring variant thereof” refers to variants of the Mcl1 polypeptide of nucleic acid sequence which exist naturally within the defined taxonomic group, such as mammalian, such as mouse, monkey, and preferably human. Typically, when referring to “naturally occurring variants” of a polynucleotide the term also may encompass any allelic variant of the Mcl1 encoding genomic DNA which is found at the Chromosome Chr 1: 148.81-148.82 Mb by chromosomal translocation or duplication, and the RNA, such as mRNA derived therefrom. “Naturally occurring variants” may also include variants derived from alternative splicing of the Mcl1 mRNA. When referenced to a specific polypeptide sequence, e.g., the term also includes naturally occurring forms of the protein which may therefore be processed, e.g. by co- or post-translational modifications, such as signal peptide cleavage, proteolytic cleavage, glycosylation, etc.

### Oligomer Sequences

The oligomers comprise or consist of a contiguous nucleotide sequence which corresponds to the reverse complement of a nucleotide sequence present in SEQ ID NO: 1 or 70. Thus, in some embodiments, the oligomer can comprise or consist of, a sequence selected from the group consisting of SEQ ID NO 2-18, SEQ ID NO 77-82 and SEQ ID NO 95-117 wherein said oligomer (or contiguous nucleotide portion thereof) may optionally have one, two, or three mismatches against said selected sequence.

In some embodiments, the oligomers comprise or consist of a contiguous nucleotide sequence which is found in a sequence of nucleobases (bases) selected from the group consisting of SEQ ID NOs 118, 119, 120, 121, 122, 123, 124, 125, 126 and 127 or a sub-sequence of at least 10 contiguous nucleotides thereof.

The oligomer may comprise or consist of a contiguous nucleotide sequence which is fully complementary (perfectly complementary) to the equivalent region of a nucleic acid which encodes a mammalian Mcl1 (e.g., SEQ ID NO: 1 or 70). The oligomer can comprise or consist of an antisense nucleotide sequence.

In some embodiments, the oligomer may tolerate 1, 2, or 3 mismatches, when hybridising to the target sequence and still sufficiently bind to the target to show the desired effect, i.e. down-regulation of the target. Mismatches may, for example, be compensated by increased length of the oligomer nucleotide sequence and/or an increased number of nucleotide analogues, such as LNA, present within the nucleotide sequence.

In some embodiments, the contiguous nucleotide sequence comprises no more than 3, such as no more than 2 mismatches when hybridizing to the target sequence, such as to the corresponding region of a nucleic acid which encodes a mammalian Mcl1.

In some embodiments, the contiguous nucleotide sequence comprises no more than a single mismatch when hybridizing to the target sequence, such as the corresponding region of a nucleic acid which encodes a mammalian Mcl1.

The nucleotide sequence of the oligomers of the invention or the contiguous nucleotide sequence is preferably at least 80% homologous to a corresponding sequence selected from the group consisting of SEQ ID NO 2-18, SEQ ID NO 77-82 and SEQ ID NO 95-117, such as at least 85%, at least 90%, at least 91%, at least 92%, at least 93%, at least 94%, at least 95%, at least 96% homologous, at least 97% homologous, at least 98% homologous, at least 99% homologous, such as 100% homologous (identical).

The nucleotide sequence of the oligomers of the invention or the contiguous nucleotide sequence is preferably at least 80% homologous to the reverse complement of a corresponding sequence present in SEQ ID NO 1 or 70, such as at least 85%, at least 90%, at least 91%, at least 92% at least 93%, at least 94%, at least 95%, at least 96% homologous, at least 97% homologous, at least 98% homologous, at least 99% homologous, such as 100% homologous (identical).

The nucleotide sequence of the oligomers of the invention or the contiguous nucleotide sequence is preferably at least 80% complementary to a sub-sequence present in SEQ ID NO: 1 or 70, such as at least 85%, at least 90%, at least 91%, at least 92%, at least 93%, at least 94%, at least 95%, at least 96% complementary, at least 97% complementary, at least 98% complementary, at least 99% complementary, such as 100% complementary (perfectly complementary).

In some embodiments the oligomer (or contiguous nucleotide portion thereof) is selected from, or comprises, one of the sequences selected from the group consisting of SEQ ID NOS: 95, 96, 97, 98, 99, 100, 101, 102, 103, 104, 105, 106, 107, 108, 109, 110, 111, 112, 113, 114, 115, 116, and 117 or SEQ ID NOs 2, 3, 4, 5, 6, 7, 8, 9, 10, 11, 12, 13, 14, 15, 16, 17 and 18, or SEQ ID NO 77, 78, 79, 80, 81 and 82; or a sub-sequence of at least 10 contiguous nucleotides thereof, wherein said oligomer (or contiguous nucleotide portion thereof) may optionally comprise one, two, or three mismatches when compared to the sequence.

In some embodiments the oligomer (or contiguous nucleotide portion thereof) is selected from, or comprises, one of the sequences selected from the group consisting of SEQ ID NOS: 118, 119, 120, 121, 122, 123, 124, 125, 126 and 127 or a sub-sequence of at least 10 contiguous nucleotides thereof, wherein said oligomer (or contiguous nucleotide portion thereof) may optionally comprise one, two, or three mismatches when compared to the said sequence.

In some embodiments the sub-sequence may consist of 11, 12, 13, 14, 15, 16, 17, 18, 19, 20, 21, 22, 23, 24, 25, 26, 27, 28, or 29 contiguous nucleotides, such as between 12-22, such as between 12-18 nucleotides. Suitably, in some embodiments, the sub-sequence is of the same length as the contiguous nucleotide sequence of the oligomer of the invention.

However, it is recognised that, in some embodiments the nucleotide sequence of the oligomer may comprise additional 5′ or 3′ nucleotides, such as, independently, 1, 2, 3, 4 or 5 additional nucleotides 5′ and/or 3′, which are non-complementary to the target sequence. In this respect the oligomer of the invention, may, in some embodiments, comprise a contiguous nucleotide sequence which is flanked 5′ and or 3′ by additional nucleotides. In some embodiments the additional 5′ or 3′ nucleotides are naturally occurring nucleotides, such as DNA or RNA. In some embodiments, the additional 5′ or 3′ nucleotides may represent region D as referred to in the context of gapmer oligomers herein.

In some embodiments, the contiguous nucleotide sequence comprises a sequence of 10-16 nucleotides. Examples of oligonucleotide sequences comprising 14, 15 or 16 nucleotides are shown in SEQ ID NOS: 2-18. Shorter sequences can be derived therefrom. Longer sequences may include all, or at least 10 nucleotides from those exemplified SEQ ID NOs.

Oligonucleotide sequences which have fewer nucleotides, such as 10, 11, 12, 13, 14, or 15, may therefore have a contiguous nucleotide sequence which corresponds to a sub-sequence, for example of at least 8, at least 9, at least 10, at least 11, at least 12, at least 13, at least 14 or 15, contiguous nucleotides found in a sequence selected from the group consisting of SEQ ID NO 2-18, SEQ ID NO 77-82 and, SEQ ID NO 95-117. Hence, shorter sequences can be derived therefrom. Longer sequences may include all, or at least 10 nucleotides from those exemplified SEQ ID NOs. Typically if only a proportion of the sequence of the oligonucleotide is present a SEQ ID selected from SEQ ID NO 2-18, SEQ ID NO 77-82 and, SEQ ID NO 95-117, then the remaining portion of the sequence of the oligonucleotide is homologous to the corresponding nucleotides flanking said SEQ ID. In some embodiments, the oligomer can comprise or consist of, a sequence selected from the group consisting of SEQ ID NO 112, 113 and 114 or a subsequence there of, such as SEQ ID NOs 77, 78 and 79, wherein said oligomer (or contiguous nucleotide portion thereof) may optionally have one, two, or three mismatches against said selected sequence. In some embodiments, the oligomer can comprise or consist of, a sequence selected from the group consisting of SEQ ID NO 113, 114 and 115 or a subsequence there of, such as SEQ ID NOs 78, 79 and 80, wherein said oligomer (or contiguous nucleotide portion thereof) may optionally have one, two, or three mismatches against said selected sequence.

When determining “homology” between the oligomers of the invention (or contiguous nucleotide sequence) and the nucleic acid which encodes the mammalian Mcl1 or the reverse complement thereof, such as those disclosed herein, the determination of homology may be made by a simple alignment with the corresponding nucleotide sequence of the compound of the invention and the corresponding region of the nucleic acid which encodes the mammalian Mcl1 (or target nucleic acid), or the reverse complement thereof, and the homology is determined by counting the number of bases which align and dividing by the total number of contiguous nucleotides in the compound of the invention, and multiplying by 100. In such a comparison, if gaps exist, it is preferable that such gaps are merely mismatches rather than areas where the number of nucleotides within the gap differ between the nucleotide sequence of the invention and the target nucleic acid.

The terms “corresponding to” and “corresponds to” refer to the comparison between the nucleotide sequence of the oligomer or contiguous nucleotide sequence (a first sequence) and the equivalent contiguous nucleotide sequence of a further sequence selected from either i) a sub-sequence of the reverse complement of the nucleic acid target, such as the mRNA which encodes the Mcl1 protein, such as SEQ ID NO: 1 or 70, and/or ii) the sequence of nucleotides provided herein such as the group consisting of SEQ ID NO 2-18, SEQ ID NO 77-82 and SEQ ID NO 95-117, or sub-sequence thereof. Nucleotide analogues are compared directly to their equivalent or corresponding nucleotides. A first sequence which corresponds to a further sequence under i) or ii) typically is identical to that sequence over the length of the first sequence (such as the contiguous nucleotide sequence) or, as described herein may, in some embodiments, is at least 80% homologous to a corresponding sequence, such as at least 85%, at least 90%, at least 91%, at least 92% at least 93%, at least 94%, at least 95%, at least 96% at least 97% homologous, at least 98% homologous, at least 99% homologous, such as 100% homologous (identical).

The terms “corresponding nucleotide analogue” and “corresponding nucleotide” are intended to indicate that the nucleotide in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine.

### Length

The oligomers comprise or consist of a contiguous nucleotide sequence of a total of between 10, 11, 12, 13, 14, 15, 16, 17, 18, 19, 20, 21, 22, 23, 24, 25, 26, 27, 28, 29 or 30 contiguous nucleotides in length.

In some embodiments, the oligomers comprise or consist of a contiguous nucleotide sequence of a total of between 10-22, such as 12-18, such as 13-17 or 12-16, such as 13, 14, 15, 16 contiguous nucleotides in length.

In some embodiments, the oligomers comprise or consist of a contiguous nucleotide sequence of a total of 10, 11, 12, 13, or 14 contiguous nucleotides in length.

In some embodiments, the oligomer according to the invention consists of no more than 22 nucleotides, such as no more than 20 nucleotides, such as no more than 18 nucleotides, such as 15, 16 or 17 nucleotides. In some embodiments the oligomer of the invention comprises less than 20 nucleotides.

In some embodiments the oligomer (or contiguous nucleotide portion thereof) is selected from, or comprises, one of the sequences selected from the group consisting of: SEQ ID NO 2, 6, 9, 10, 13, 14, 15, 16 and/or 17, 77, 78, 79, 80, 81, and 82 or a sub-sequence of at least 10 contiguous nucleotides thereof, such as 11, 12, 13, 14, 15, or 16 contiguous nucleotides thereof, wherein said oligomer (or contiguous nucleotide portion thereof) may optionally comprise one, two, or three mismatches against said selected sequence.

In some embodiments the oligomer consists or comprises of a sequence which is, or corresponds to, a sequence selected from the group consisting of: SEQ ID NO 78 or 113, such as SEQ ID NO 90 or 91.

In some embodiments the oligomer consists or comprises of a sequence which is, or corresponds to, a sequence selected from the group consisting of: SEQ ID NO 16 or 109, such as SEQ ID NO 64.

As shown herein, in some exemplary aspects, oligomers of the invention, for example SEQ ID NO: 64, 91 and 90, show pronounced down-regulation of Mcl-1 mRNAs in various cell lines using natural uptake as well as in murine liver tissue. In the human chronic myelogenous leukemia K562 cell line, the human Burkitt's lymphoma cancer Namalwa cell line and the human hepatocyte carcinoma HepG2 cell line, SEQ ID NO: 64, 90 and 91, show induction of caspase 3/7 activity as well as reduced cell viability in concordance with the potency on Mcl-1 mRNA down-regulation. In some embodiments the oligomers of the invention is able to induce apoptosis in a cell into which it is introduced (e.g. in an effective amount), such as a cancer cell, such as the cell lines herein referred to (such as in the caspase assay herein disclosed). Some oligomers which we have found may be effective in inducing caspase activity include SEQ ID NO 2, SEQ ID NO 36, SEQ ID NO 77, SEQ ID NO 66, SEQ ID 17 and SEQ ID NO 92. Further such oligomers are illustrated in the examples.

In some embodiments the oligomer consists or comprises of a sequence which is, or corresponds to, a sequence selected from the group consisting of: SEQ ID NO 2, SEQ ID NO 36, SEQ ID NO 37, and SEQ ID NO 38, or a contiguous sequence of at least 12, 13, 14, 15, or 16 consecutive nucleotides present in said sequence, wherein the nucleotides present in the oligomer may be substituted with a corresponding nucleotide analogue, wherein said oligomer may optionally comprise one, two, or three mismatches against said selected sequence.

In some embodiments the oligomer consists or comprises of a sequence which is, or corresponds to, a sequence selected from the group consisting of: SEQ ID NO 77, SEQ ID NO 78, SEQ ID NO 79, SEQ ID NO 80, SEQ ID NO 81, SEQ ID NO 82, or a contiguous sequence of at least 12, 13, 14, 15, or 16 consecutive nucleotides present in said sequence, wherein the nucleotides present in the oligomer may be substituted with a corresponding nucleotide analogue, wherein said oligomer may optionally comprise one, two, or three mismatches against said selected sequence.

In some embodiments the oligomer of the invention may not induce apoptosis, for example as determined by the caspase assay. In this regards, whilst apoptosis may be desirable for the efficient killing of cells, e.g. cancer cells, it may, in some embodiments, be negatively associated to a more toxic effect on non-target cells and tissues. As such, depending upon the medical indication to be treated, it may be beneficial to select oligonucleotides which are very effective at triggering apoptosis, whilst others, where perhaps the medical indication is not immediately life threatening, an oligomer which is not as potent in triggering apoptosis may be appropriate.

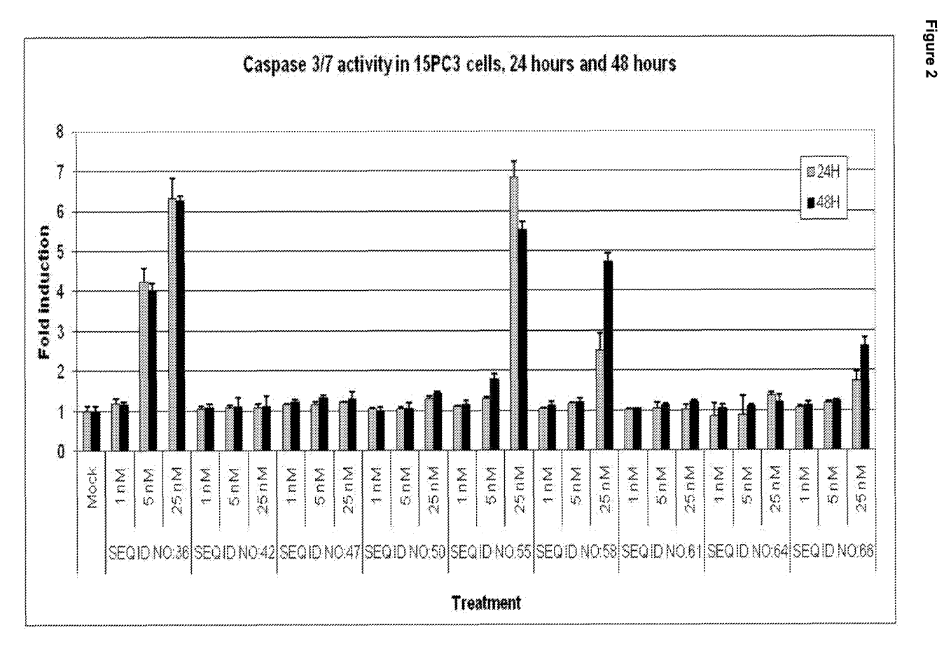

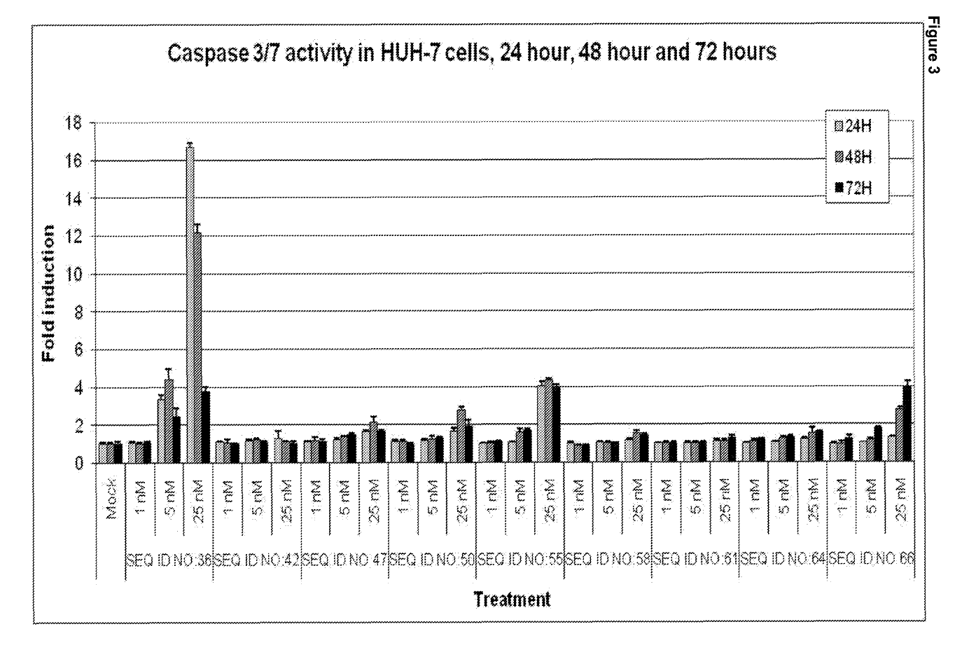

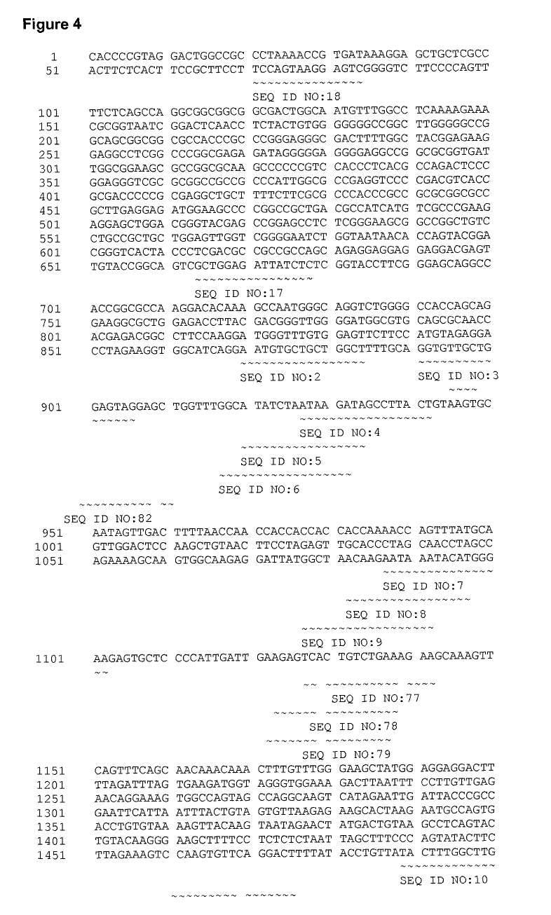

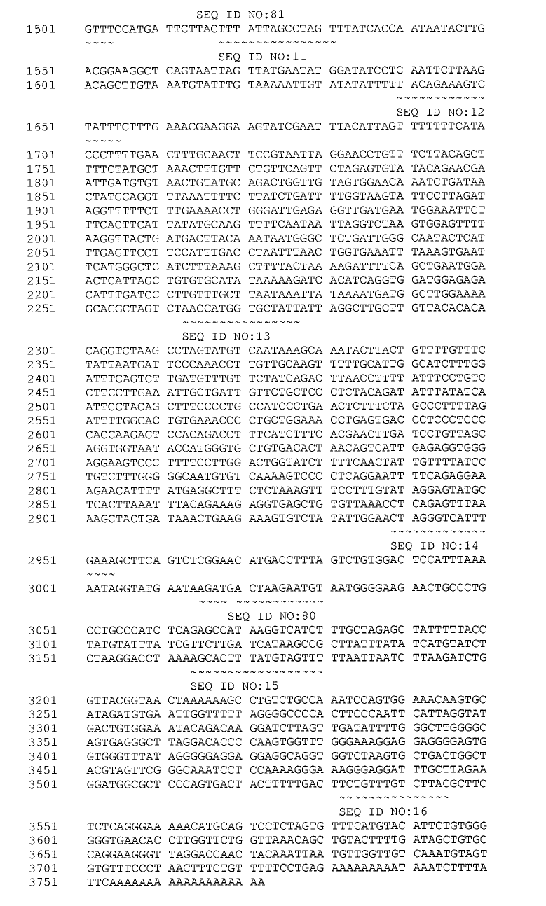

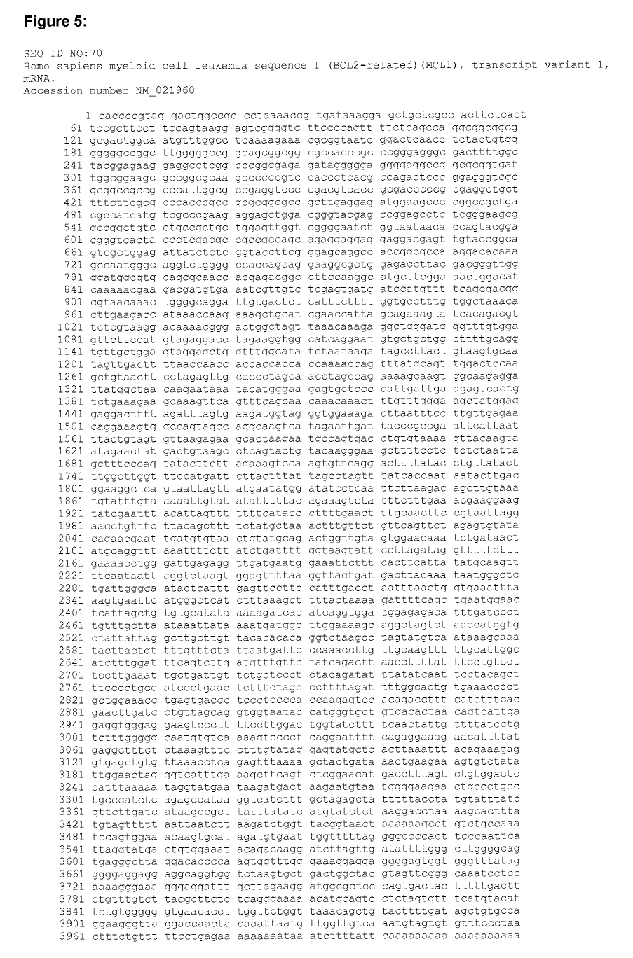

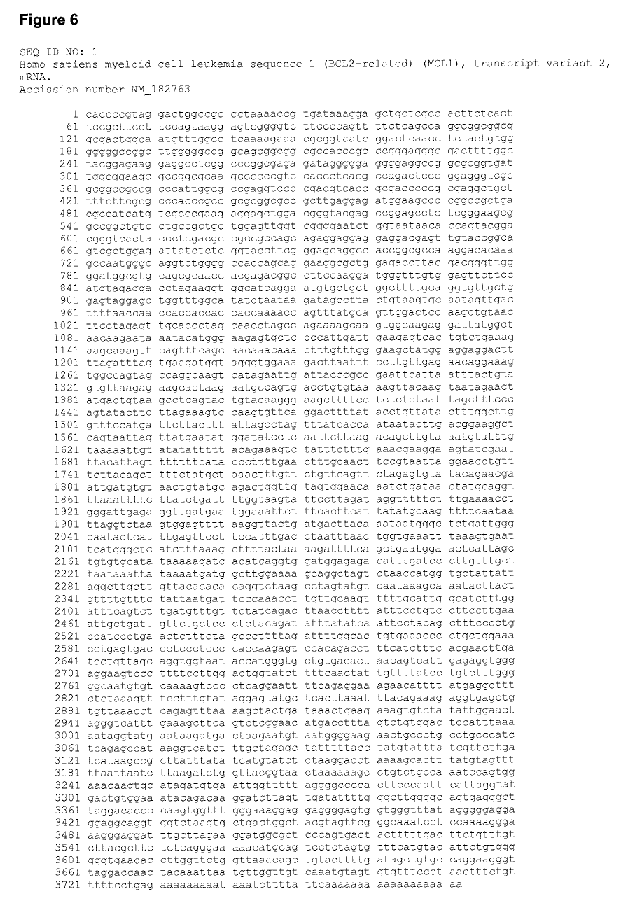

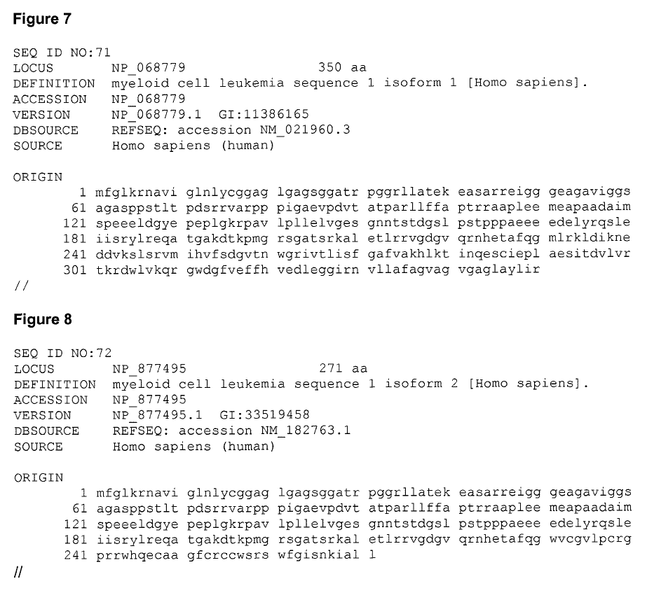

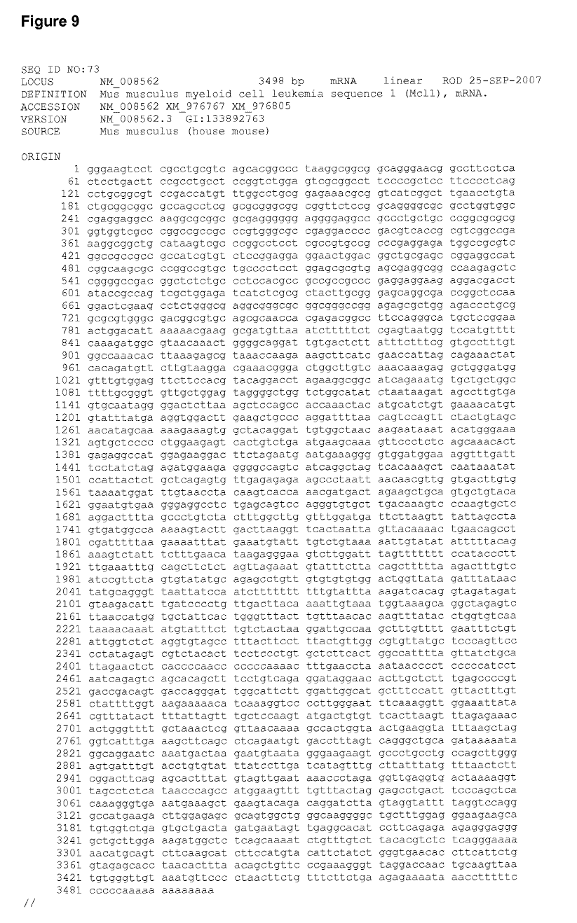

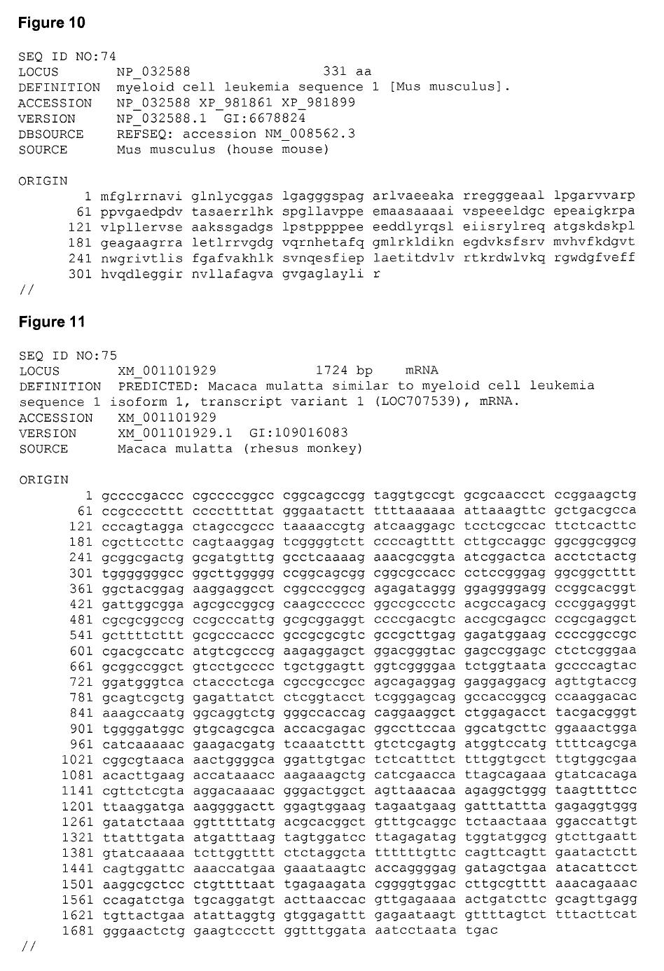

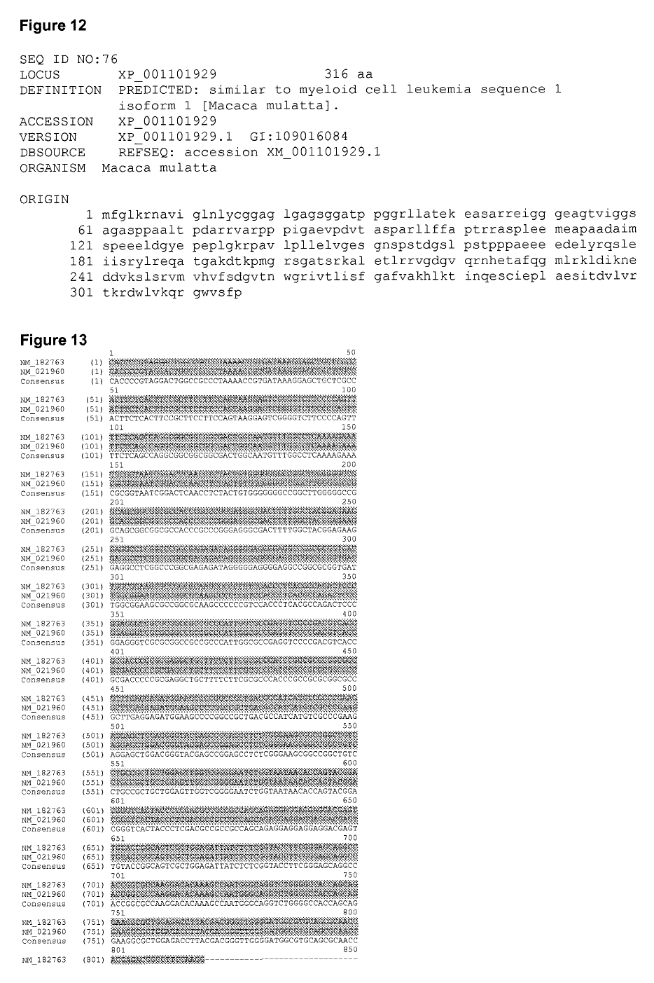

As shown in FIGS. 2 and 3, LNA oligomers such as SEQ ID No 36 are remarkably effective at triggering apoptosis in proliferating cells, and as such may be considered preferable. Therefore in some embodiments where the contiguous nucleotides sequence, or a sub-sequence thereof, is found within SEQ ID NO 95 or 2, such as oligomers 36, 37 or 38. Such oligomers may preferably comprise LNA units.

In some embodiments the oligomer according to the invention consists or comprises of a contiguous nucleotide sequence according to SEQ ID NO 2 or 95, such as SEQ ID NO 36, 37 and 38; or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 3 or 96, such as SEQ ID 39, or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 4 or 97, such as SEQ ID NO 40, or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 5 or 98, such as SEQ ID NO 41, or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 6 or 99, such as SEQ ID NO 42, 43 and 44; or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 7 or 100, such as SEQ ID NO 45, or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 8 or 101, such as SEQ ID 46, or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 9 or 102, such as SEQ ID 47, 48 and 49; or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 10 or 103, such as SEQ ID 50, 51 and 52; or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 11 or 104, such as SEQ ID 53, or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 12 or 105, such as SEQ ID 54, or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 13 or 106, such as SEQ ID 55, 56 and 57; or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 14 or 107, such as SEQ ID 58, 59 and 60; or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 15 or 108, such as SEQ ID 61, 62 and 63; or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 16 or 109, such as SEQ ID 64 and 65; or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 17 or 110, such as SEQ ID 66, 67 and 68; or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 18 or 111, such as SEQ ID 69, or a sub-sequence of at least 10 contiguous nucleotides thereof, such as 11, 12, 13, 14, 15 or 16 contiguous nucleotides thereof.

In some embodiments the oligomer according to the invention consists or comprises of a nucleotide sequence according to SEQ ID 77 or 112, such as 11, 12, 13, 14, 15 or 16 contiguous nucleotides thereof.

In some embodiments the oligomer according to the invention consists or comprises of a nucleotide sequence according to SEQ ID 78 or 113, such as 11, 12, 13, 14, 15 or 16 contiguous nucleotides thereof.

In some embodiments the oligomer according to the invention consists or comprises of a nucleotide sequence according to SEQ ID 79 or 114, such as 11, 12, 13, 14, 15 or 16 contiguous nucleotides thereof.

In some embodiments the oligomer according to the invention consists or comprises of a nucleotide sequence according to SEQ ID 80 or 115, such as 11, 12, 13, 14, 15 or 16 contiguous nucleotides thereof.

In some embodiments the oligomer according to the invention consists or comprises of a nucleotide sequence according to SEQ ID 81 or 116, such as 11, 12, 13, 14, 15 or 16 contiguous nucleotides thereof.

In some embodiments the oligomer according to the invention consists or comprises of a nucleotide sequence according to SEQ ID 82 or 117, such as 11, 12, 13, 14, 15 or 16 contiguous nucleotides thereof.

### Nucleotide Analogues

The term “nucleotide” as used herein, refers to a glycoside comprising a sugar moiety, a base moiety and a covalently linked phosphate group and covers both naturally occurring nucleotides, such as DNA or RNA, preferably DNA, and non-naturally occurring nucleotides comprising modified sugar and/or base moieties, which are also referred to as “nucleotide analogues” herein.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1:

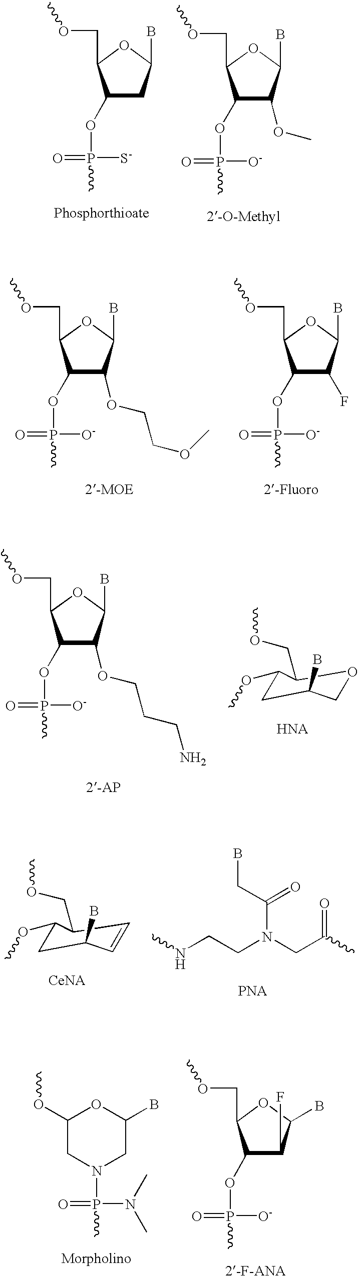

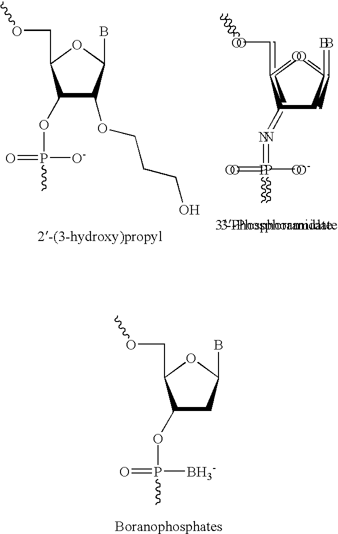

The oligomer may thus comprise or consist of a simple sequence of natural occurring nucleotides—preferably 2′-deoxynucleotides (referred to here generally as “DNA”), but also possibly ribonucleotides (referred to here generally as “RNA”), or a combination of such naturally occurring nucleotides and one or more non-naturally occurring nucleotides, i.e. nucleotide analogues. Such nucleotide analogues may suitably enhance the affinity of the oligomer for the target sequence.

Examples of suitable and preferred nucleotide analogues are provided by PCT/DK2006/000512 or are referenced therein.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

In some embodiments the oligomer comprises at least 2 nucleotide analogues. In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA.

It will be recognised that when referring to a preferred nucleotide sequence motif or nucleotide sequence, which consists of only nucleotides, the oligomers of the invention which are defined by that sequence may comprise a corresponding nucleotide analogue in place of one or more of the nucleotides present in said sequence, such as LNA units or other nucleotide analogues, which raise the duplex stability/Tₘ of the oligomer/target duplex (i.e. affinity enhancing nucleotide analogues).

In some embodiments, any mismatches between the nucleotide sequence of the oligomer and the target sequence are preferably found in regions outside the affinity enhancing nucleotide analogues, such as region B as referred to herein, and/or region D as referred to herein, and/or at the site of non modified such as DNA nucleotides in the oligonucleotide, and/or in regions which are 5′ or 3′ to the contiguous nucleotide sequence.

Examples of such modification of the nucleotide include modifying the sugar moiety to provide a 2′-substituent group or to produce a bridged (locked nucleic acid) structure which enhances binding affinity and may also provide increased nuclease resistance.

A preferred nucleotide analogue is LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). Most preferred is beta-D-oxy-LNA.

In some embodiments the nucleotide analogues present within the oligomer of the invention (such as in regions A and C mentioned herein) are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid-Christensen, 2002. Nucl. Acids. Res. 2002 30: 4918-4925, hereby incorporated by reference) units and 2′MOE units. In some embodiments there is only one of the above types of nucleotide analogues present in the oligomer of the invention, or contiguous nucleotide sequence thereof.

In some embodiments the nucleotide analogues are 2′-O-methoxyethyl-RNA (2′MOE), 2′-fluoro-DNA monomers or LNA nucleotide analogues, and as such the oligonucleotide of the invention may comprise nucleotide analogues which are independently selected from these three types of analogue, or may comprise only one type of analogue selected from the three types. In some embodiments at least one of said nucleotide analogues is 2′-MOE-RNA, such as 2, 3, 4, 5, 6, 7, 8, 9 or 10 2′-MOE-RNA nucleotide units. In some embodiments at least one of said nucleotide analogues is 2′-fluoro DNA, such as 2, 3, 4, 5, 6, 7, 8, 9 or 10 2′-fluoro-DNA nucleotide units.

In some embodiments, the oligomer according to the invention comprises at least one Locked Nucleic Acid (LNA) unit, such as 1, 2, 3, 4, 5, 6, 7, or 8 LNA units, such as between 3-7 or 4 to 8 LNA units, or 3, 4, 5, 6 or 7 LNA units. In some embodiments, all the nucleotide analogues are LNA. In some embodiments, the oligomer may comprise both beta-D-oxy-LNA, and one or more of the following LNA units: thio-LNA, amino-LNA, oxy-LNA, and/or ENA in either the beta-D or alpha-L configurations or combinations thereof. In some embodiments all LNA cytosine units are 5′ methyl-Cytosine. In some embodiments of the invention, the oligomer may comprise both LNA and DNA units. Preferably the combined total of LNA and DNA units is 10-25, preferably 10-20, even more preferably 12-16. In some embodiments of the invention, the nucleotide sequence of the oligomer, such as the contiguous nucleotide sequence consists of at least one LNA and the remaining nucleotide units are DNA units. In some embodiments the oligomer comprises only LNA nucleotide analogues and naturally occurring nucleotides (such as RNA or DNA, most preferably DNA nucleotides), optionally with modified internucleotide linkages such as phosphorothioate.

The term “nucleobase” refers to the base moiety of a nucleotide and covers both naturally occurring a well as non-naturally occurring variants. Thus, “nucleobase” covers not only the known purine and pyrimidine heterocycles but also heterocyclic analogues and tautomeres thereof.

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

In some embodiments, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

It should be recognised that, in some aspects, the term nucleobase may also be used to refer to a nucleotide which may be either naturally occurring or non-naturally occurring—in this respect the term nucleobase and nucleotide are, in some embodiments used interchangeably herein.

### LNA

The term “LNA” refers to a bicyclic nucleotide analogue, known as “Locked Nucleic Acid”. It may refer to an LNA monomer, or, when used in the context of an “LNA oligonucleotide” refers to an oligonucleotide containing one or more such bicyclic nucleotide analogues.

The LNA used in the oligonucleotide compounds of the invention preferably has the structure of the general formula I

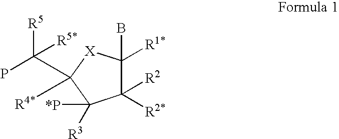

wherein X is selected from —O—, —S—, —N(R^{N}*)—, —C(R⁶R⁶*)—;

B is selected from hydrogen, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands;

P designates the radical position for an internucleotide linkage to a succeeding monomer, or a 5′-terminal group, such internucleotide linkage or 5′-terminal group optionally including the substituent R⁵ or equally applicable the substituent R⁵*;

P* designates an internucleotide linkage to a preceding monomer, or a 3′-terminal group;

R⁴* and R²* together designate a biradical consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z,

wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), and each of the substituents R¹*, R², R³, R⁵, R⁵*, R⁶ and R⁶*, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof;

In some embodiments R⁵* is selected from H, —CH₃, —CH₂—CH₃, —CH₂—O—CH₃, and —CH═CH₂.

In some embodiments, R⁴* and R²* together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O—, and —C(R^{a}R^{b})—S—, —C(R^{a}R^{b})—C(R^{c}R^{d})—S—, wherein R^{a}, R^{b}, R^{c}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂),

In a further embodiment R⁴* and R²* together designate a biradical (bivalent group) selected from —CH₂—O—, —CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, —CH(CH₂—O—CH₃)—O—.

For all chiral centers, asymmetric groups may be found in either R or S orientation.

Preferably, the LNA used in the oligomer of the invention comprises at least one LNA unit according to any of the formulas

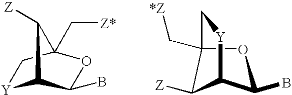

wherein Y is —O—, —O—CH₂—, —S—, —NH—, or N(R^{H}); Z and Z* are independently selected among an internucleotide linkage, a terminal group or a protecting group; B constitutes a natural or non-natural nucleotide base moiety, and R^{H} is selected from hydrogen and C₁₋₄-alkyl.

Specifically preferred LNA units are shown in scheme 2:

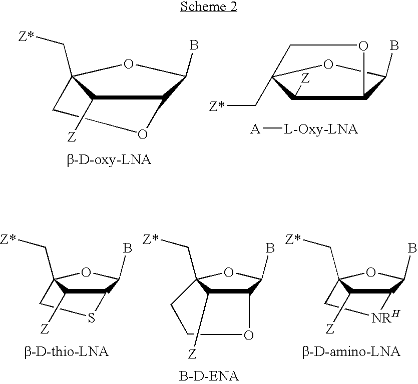

The term “thio-LNA” comprises a locked nucleotide in which Y in the general formula above is selected from S or —CH₂—S—. Thio-LNA can be in both beta-D and alpha-L-configuration.

The term “amino-LNA” comprises a locked nucleotide in which Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which Y in the general formula above represents —O— or —CH₂—O—. Oxy-LNA can be in both beta-D and alpha-L-configuration.

The term “ENA” comprises a locked nucleotide in which Y in the general formula above is —CH₂—O— (where the oxygen atom of —CH₂—O— is attached to the 2′-position relative to the base B).

In a preferred embodiment LNA is selected from beta-D-oxy-LNA, alpha-L-oxy-LNA, beta-D-amino-LNA and beta-D-thio-LNA, in particular beta-D-oxy-LNA.

### RNAse Recruitment

It is recognised that an oligomeric compound may function via non RNase mediated degradation of target mRNA, such as by steric hindrance of translation, or other methods, however, the preferred oligomers of the invention are capable of recruiting an endoribonuclease (RNase), such as RNase H.

It is preferable that the oligomer, or contiguous nucleotide sequence, comprises of a region of at least 6, such as at least 7 consecutive nucleotide units, such as at least 8 or at least 9 consecutive nucleotide units (residues), including 7, 8, 9, 10, 11, 12, 13, 14, 15 or 16 consecutive nucleotides, which, when formed in a duplex with the complementary target RNA is capable of recruiting RNase. The contiguous sequence which is capable of recruiting RNAse may be region B as referred to in the context of a gapmer as described herein. In some embodiments the size of the contiguous sequence which is capable of recruiting RNAse, such as region B, may be higher, such as 10, 11, 12, 13, 14, 15, 16, 17, 18, 19 or 20 nucleotide units.

EP 1 222 309 provides in vitro methods for determining RNaseH activity, which may be used to determine the ability to recruit RNaseH. A oligomer is deemed capable of recruiting RNase H if, when provided with the complementary RNA target, it has an initial rate, as measured in pmol/l/min, of at least 1%, such as at least 5%, such as at least 10% or less than 20% of the equivalent DNA only oligonucleotide, with no 2′ substitutions, with phosphorothioate linkage groups between all nucleotides in the oligonucleotide, using the methodology provided by Example 91-95 of EP 1 222 309.

In some embodiments, an oligomer is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

In other embodiments, an oligomer is deemed capable of recruiting RNaseH if, when provided with the complementary RNA target, and RNaseH, the RNaseH initial rate, as measured in pmol/l/min, is at least 20%, such as at least 40%, such as at least 60%, such as at least 80% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

Typically the region of the oligomer which forms the consecutive nucleotide units which, when formed in a duplex with the complementary target RNA is capable of recruiting RNase consists of nucleotide units which form a DNA/RNA like duplex with the RNA target—and include both DNA units and LNA units which are in the alpha-L configuration, particularly preferred being alpha-L-oxy LNA.

The oligomer of the invention may comprise a nucleotide sequence which comprises both nucleotides and nucleotide analogues, and may be in the form of a gapmer, a headmer or a mixmer.

A headmer is defined by a contiguous stretch of non-RNase recruiting nucleotide analogues at the 5′-end followed by a contiguous stretch of DNA or modified nucleotide units recognizable and cleavable by the RNase towards the 3′-end (such as at least 7 such nucleotides), and a tailmer is defined by a contiguous stretch of DNA or modified nucleotides recognizable and cleavable by the RNase at the 5′-end (such as at least 7 such nucleotides), followed by a contiguous stretch of non-RNase recruiting nucleotide analogues towards the 3′-end. Other chimeras according to the invention, called mixmers consisting of an alternate composition of DNA or modified nucleotides recognizable and cleavable by RNase and non-RNase recruiting nucleotide analogues. Some nucleotide analogues may also be able to mediate RNaseH binding and cleavage. Since α-L-LNA recruits RNaseH activity to a certain extent, smaller gaps of DNA or modified nucleotides recognizable and cleavable by the RNaseH for the gapmer construct might be required, and more flexibility in the mixmer construction might be introduced.

### Gapmer Design

Preferably, the oligomer of the invention is a gapmer. A gapmer oligomer is an oligomer which comprises a contiguous stretch of nucleotides which is capable of recruiting an RNAse, such as RNAseH, such as a region of at least 6 or 7 DNA nucleotides, referred to herein in as region B, wherein region B is flanked both 5′ and 3′ by regions of affinity enhancing nucleotide analogues, such as between 1-6 nucleotide analogues 5′ and 3′ to the contiguous stretch of nucleotides which is capable of recruiting RNAse—these regions are referred to as regions A and C respectively.

Preferably the gapmer comprises a (poly)nucleotide sequence of formula (5′ to 3′), A-B-C, or optionally A-B-C-D or D-A-B-C, wherein; region A (5′ region) consists or comprises of at least one nucleotide analogue, such as at least one LNA unit, such as between 1-6 nucleotide analogues, such as LNA units, and; region B consists or comprises of at least five consecutive nucleotides which are capable of recruiting RNAse (when formed in a duplex with a complementary RNA molecule, such as the mRNA target), such as DNA nucleotides, and; region C (3′ region) consists or comprises of at least one nucleotide analogue, such as at least one LNA unit, such as between 1-6 nucleotide analogues, such as LNA units, and; region D, when present consists or comprises of 1, 2 or 3 nucleotide units, such as DNA nucleotides.

In some embodiments, region A consists of 1, 2, 3, 4, 5 or 6 nucleotide analogues, such as LNA units, such as between 2-5 nucleotide analogues, such as 2-5 LNA units, such as 3 or 4 nucleotide analogues, such as 3 or 4 LNA units; and/or region C consists of 1, 2, 3, 4, 5 or 6 nucleotide analogues, such as LNA units, such as between 2-5 nucleotide analogues, such as 2-5 LNA units, such as 3 or 4 nucleotide analogues, such as 3 or 4 LNA units.

In some embodiments B consists or comprises of 5, 6, 7, 8, 9, 10, 11 or 12 consecutive nucleotides which are capable of recruiting RNAse, or between 6-10, or between 7-9, such as 8 consecutive nucleotides which are capable of recruiting RNAse. In some embodiments region B consists or comprises at least one DNA nucleotide unit, such as 1-12 DNA units, preferably between 4-12 DNA units, more preferably between 6-10 DNA units, such as between 7-10 DNA units, most preferably 8, 9 or 10 DNA units.

In some embodiments region A consist of 3 or 4 nucleotide analogues, such as LNA, region B consists of 7, 8, 9 or 10 DNA units, and region C consists of 3 or 4 nucleotide analogues, such as LNA. Such designs include (A-B-C) 3-10-3, 3-10-4, 4-10-3, 3-9-3, 3-9-4, 4-9-3, 3-8-3, 3-8-4, 4-8-3, 3-7-3, 3-7-4, 4-7-3, and may further include region D, which may have one or 2 nucleotide units, such as DNA units.

Further gapmer designs are disclosed in WO2004/046160 and are hereby incorporated by reference.

US provisional application, 60/977,409, hereby incorporated by reference, refers to ‘shortmer’ gapmer oligomers, which, in some embodiments may be the gapmer oligomer according to the present invention.

In some embodiments the oligomer is consisting of a contiguous nucleotide sequence of a total of 10, 11, 12, 13 or 14 nucleotide units, wherein the contiguous nucleotide sequence is of formula (5′-3′), A-B-C, or optionally A-B-C-D or D-A-B-C, wherein; A consists of 1, 2 or 3 nucleotide analogue units, such as LNA units; B consists of 7, 8 or 9 contiguous nucleotide units which are capable of recruiting RNAse when formed in a duplex with a complementary RNA molecule (such as a mRNA target); and C consists of 1, 2 or 3 nucleotide analogue units, such as LNA units. When present, D consists of a single DNA unit.

In some embodiments A consists of 1 LNA unit. In some embodiments A consists of 2 LNA units. In some embodiments A consists of 3 LNA units. In some embodiments C consists of 1 LNA unit. In some embodiments C consists of 2 LNA units. In some embodiments C consists of 3 LNA units. In some embodiments B consists of 7 nucleotide units. In some embodiments B consists of 8 nucleotide units. In some embodiments B consists of 9 nucleotide units. In some embodiments B comprises of between 1-9 DNA units, such as 2, 3, 4, 5, 6, 7 or 8 DNA units. In some embodiments B consists of DNA units. In some embodiments B comprises of at least one LNA unit which is in the alpha-L configuration, such as 2, 3, 4, 5, 6, 7, 8 or 9 LNA units in the alpha-L-configuration. In some embodiments B comprises of at least one alpha-L-oxy LNA unit or wherein all the LNA units in the alpha-L-configuration are alpha-L-oxy LNA units. In some embodiments the number of nucleotides present in A-B-C are selected from the group consisting of (nucleotide analogue units—region B—nucleotide analogue units): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, 3-10-1. In some embodiments the number of nucleotides in A-B-C are selected from the group consisting of: 2-7-1, 1-7-2, 2-7-2, 3-7-3, 2-7-3, 3-7-2, 3-7-4, and 4-7-3. In some embodiments both A and C consists of two LNA units each, and B consists of 8 or 9 nucleotide units, preferably DNA units.

### Internucleotide Linkages

The terms “linkage group” or “internucleotide linkage” are intended to mean a group capable of covalently coupling together two nucleotides, two nucleotide analogues, and a nucleotide and a nucleotide analogue, etc. Specific and preferred examples include phosphate groups and phosphorothioate groups.

The nucleotides of the oligomer of the invention or contiguous nucleotides sequence thereof are coupled together via linkage groups. Suitably each nucleotide is linked to the 3′ adjacent nucleotide via a linkage group.

Suitable internucleotide linkages include those listed within PCT/DK2006/000512, for example the internucleotide linkages listed on the first paragraph of page 34 of PCT/DK2006/000512 (hereby incorporated by reference).

It is, in some embodiments, preferred to modify the internucleotide linkage from its normal phosphodiester to one that is more resistant to nuclease attack, such as phosphorothioate or boranophosphate—these two, being cleavable by RNase H, also allow that route of antisense inhibition in reducing the expression of the target gene.

Suitable sulphur (S) containing internucleotide linkages as provided herein may be preferred. Phosphorothioate internucleotide linkages are also preferred, particularly for the gap region (B) of gapmers. Phosphorothioate linkages may also be used for the flanking regions (A and C, and for linking A or C to D, and within region D, as appropriate).

Regions A, B and C, may however comprise internucleotide linkages other than phosphorothioate, such as phosphodiester linkages, particularly, for instance when the use of nucleotide analogues protects the internucleotide linkages within regions A and C from endo-nuclease degradation—such as when regions A and C comprise LNA nucleotides.

The internucleotide linkages in the oligomer may be phosphodiester, phosphorothioate or boranophosphate so as to allow RNase H cleavage of targeted RNA. Phosphorothioate is preferred, for improved nuclease resistance and other reasons, such as ease of manufacture.

In one aspect of the oligomer of the invention, the nucleotides and/or nucleotide analogues are linked to each other by means of phosphorothioate groups.

It is recognised that the inclusion of phosphodiester linkages, such as one or two linkages, into an otherwise phosphorothioate oligomer, particularly between or adjacent to nucleotide analogue units (typically in region A and or C) can modify the bioavailability and/or bio-distribution of an oligomer—see WO2008/053314, hereby incorporated by reference.

In some embodiments, such as the embodiments referred to above, where suitable and not specifically indicated, all remaining linkage groups are either phosphodiester or phosphorothioate, or a mixture thereof.

In some embodiments all the internucleotide linkage groups are phosphorothioate. When referring to specific gapmer oligonucleotide sequences, such as those provided herein it will be understood that, in various embodiments, when the linkages are phosphorothioate linkages, alternative linkages, such as those disclosed herein may be used, for example phosphate (phosphodiester) linkages may be used, particularly for linkages between nucleotide analogues, such as LNA, units. Likewise, when referring to specific gapmer oligonucleotide sequences, such as those provided herein, when the C residues are annotated as 5′ methyl modified cytosine, in various embodiments, one or more of the Cs present in the oligomer may be unmodified C residues.

### Oligomeric Compounds

The oligomers of the invention may, in some exemplary embodiments, be selected from the group consisting of: SEQ ID NO 19-35 and 36-69, 89-94, and 118-127.

### Conjugates

In the context the term “conjugate” is intended to indicate a heterogenous molecule formed by the covalent attachment (“conjugation”) of the oligomer as described herein to one or more non-nucleotide, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol.

Therefore, in various embodiments, the oligomer of the invention may comprise both a polynucleotide region which typically consists of a contiguous sequence of nucleotides, and a further non-nucleotide region. When referring to the oligomer of the invention consisting of a contiguous nucleotide sequence, the compound may comprise non-nucleotide components, such as a conjugate component.

In various embodiments of the invention the oligomeric compound is linked to ligands/conjugates, which may be used, e.g. to increase the cellular uptake of oligomeric compounds. WO2007/031091 provides suitable ligands and conjugates, which are hereby incorporated by reference.

The invention also provides for a conjugate comprising the compound according to the invention as herein described, and at least one non-nucleotide or non-polynucleotide moiety covalently attached to said compound. Therefore, in various embodiments where the compound of the invention consists of a specified nucleic acid or nucleotide sequence, as herein disclosed, the compound may also comprise at least one non-nucleotide or non-polynucleotide moiety (e.g. not comprising one or more nucleotides or nucleotide analogues) covalently attached to said compound.

Conjugation (to a conjugate moiety) may enhance the activity, cellular distribution or cellular uptake of the oligomer of the invention. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

The oligomers of the invention may also be conjugated to active drug substances, for example, aspirin, ibuprofen, a sulfa drug, an antidiabetic, an antibacterial or an antibiotic.

In certain embodiments the conjugated moiety is a sterol, such as cholesterol.

In various embodiments, the conjugated moiety comprises or consists of a positively charged polymer, such as a positively charged peptides of, for example between 1-50, such as 2-20 such as 3-10 amino acid residues in length, and/or polyalkylene oxide such as polyethylglycol(PEG) or polypropylene glycol—see WO 2008/034123, hereby incorporated by reference. Suitably the positively charged polymer, such as a polyalkylene oxide may be attached to the oligomer of the invention via a linker such as the releasable linker described in WO 2008/034123.

By way of example, the following conjugate moieties may be used in the conjugates of the invention:

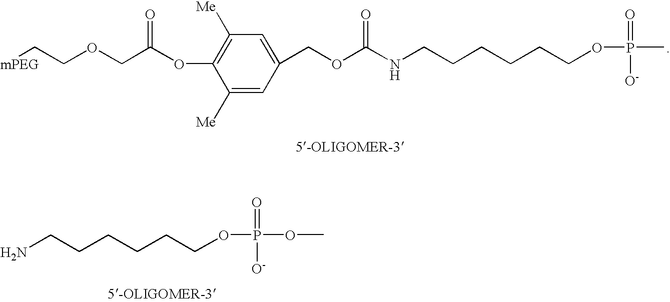

### Activated Oligomers

The term “activated oligomer,” as used herein, refers to an oligomer of the invention that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that is preferably hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl. In some embodiments, the functional moiety is self-cleaving. In other embodiments, the functional moiety is biodegradable. See e.g., U.S. Pat. No. 7,087,229, which is incorporated by reference herein in its entirety.

In some embodiments, oligomers of the invention are functionalized at the 5′ end in order to allow covalent attachment of the conjugated moiety to the 5′ end of the oligomer. In other embodiments, oligomers of the invention can be functionalized at the 3′ end. In still other embodiments, oligomers of the invention can be functionalized along the backbone or on the heterocyclic base moiety. In yet other embodiments, oligomers of the invention can be functionalized at more than one position independently selected from the 5′ end, the 3′ end, the backbone and the base.

In some embodiments, activated oligomers of the invention are synthesized by incorporating during the synthesis one or more monomers that is covalently attached to a functional moiety. In other embodiments, activated oligomers of the invention are synthesized with monomers that have not been functionalized, and the oligomer is functionalized upon completion of synthesis. In some embodiments, the oligomers are functionalized with a hindered ester containing an aminoalkyl linker, wherein the alkyl portion has the formula (CH₂)_{w}, wherein w is an integer ranging from 1 to 10, preferably about 6, wherein the alkyl portion of the alkylamino group can be straight chain or branched chain, and wherein the functional group is attached to the oligomer via an ester group (—O—C(O)—(CH₂)_{w}NH).

In other embodiments, the oligomers are functionalized with a hindered ester containing a (CH₂)_{w}-sulfhydryl (SH) linker, wherein w is an integer ranging from 1 to 10, preferably about 6, wherein the alkyl portion of the alkylamino group can be straight chain or branched chain, and wherein the functional group attached to the oligomer via an ester group (—O—C(O)—(CH₂)_{w}SH).

In some embodiments, sulfhydryl-activated oligonucleotides are conjugated with polymer moieties such as polyethylene glycol or peptides (via formation of a disulfide bond).

Activated oligomers containing hindered esters as described above can be synthesized by any method known in the art, and in particular by methods disclosed in PCT Publication No. WO 2008/034122 and the examples therein, which is incorporated herein by reference in its entirety.

In still other embodiments, the oligomers of the invention are functionalized by introducing sulfhydryl, amino or hydroxyl groups into the oligomer by means of a functionalizing reagent substantially as described in U.S. Pat. Nos. 4,962,029 and 4,914,210, i.e., a substantially linear reagent having a phosphoramidite at one end linked through a hydrophilic spacer chain to the opposing end which comprises a protected or unprotected sulfhydryl, amino or hydroxyl group. Such reagents primarily react with hydroxyl groups of the oligomer. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers of the invention have a functionalizing reagent coupled to a hydroxyl group on the backbone of the oligomer. In yet further embodiments, the oligomer of the invention is functionalized with more than one of the functionalizing reagents as described in U.S. Pat. Nos. 4,962,029 and 4,914,210, incorporated herein by reference in their entirety. Methods of synthesizing such functionalizing reagents and incorporating them into monomers or oligomers are disclosed in U.S. Pat. Nos. 4,962,029 and 4,914,210.

In some embodiments, the 5′-terminus of a solid-phase bound oligomer is functionalized with a dienyl phosphoramidite derivative, followed by conjugation of the deprotected oligomer with, e.g., an amino acid or peptide via a Diels-Alder cycloaddition reaction.

In various embodiments, the incorporation of monomers containing 2′-sugar modifications, such as a 2′-carbamate substituted sugar or a 2′-(O-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al., Tetrahedron Letters, 1991, 34, 7171.

In still further embodiments, the oligomers of the invention may have amine-containing functional moieties on the nucleobase, including on the N6 purine amino groups, on the exocyclic N2 of guanine, or on the N4 or 5 positions of cytosine. In various embodiments, such functionalization may be achieved by using a commercial reagent that is already functionalized in the oligomer synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.). In some embodiments

### Compositions

The oligomer of the invention may be used in pharmaceutical formulations and compositions. Suitably, such compositions comprise a pharmaceutically acceptable diluent, carrier, salt or adjuvant. PCT/DK2006/000512 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in PCT/DK2006/000512—which are also hereby incorporated by reference.

### Applications

The oligomers of the invention may be utilized as research reagents for, for example, diagnostics, therapeutics and prophylaxis.

In research, such oligomers may be used to specifically inhibit the synthesis of Mcl-1 protein (typically by degrading or inhibiting the mRNA and thereby prevent protein formation) in cells and experimental animals thereby facilitating functional analysis of the target or an appraisal of its usefulness as a target for therapeutic intervention.

In diagnostics the oligomers may be used to detect and quantitate Mcl-1 expression in cell and tissues by Northern blotting, in-situ hybridisation or similar techniques.

For therapeutics, an animal or a human, suspected of having a disease or disorder, which can be treated by modulating the expression of Mcl-1 is treated by administering antisense compounds in accordance with this invention. Further provided are methods of treating an animal, such as mouse or rat, or treating a human, suspected of having or being prone to a disease disorder or condition, such as those referred to herein, or those associated with expression of Mcl-1, by administering a therapeutically or prophylactically effective amount of one or more of the oligomers, conjugates or compositions of the invention.

The pharmaceutical composition according to the invention may be used for the treatment of conditions, diseases or disorders, such as those referred to herein, or those associated with expression of abnormal levels of Mcl-1.

The disease or disorder may be selected from the group consisting of; cell (hyper) proliferation disorders, such as cancer or mastocytosis.

In some embodiments the cancer may be selected from the group consisting of leukaemia, chronic myeloid leukemia, chronic lymphocytic leukemia (CLL), melanoma, multiple myeloma, hepatocellular carcinoma and acute lymphoid leukemia (ALL).

In some embodiments, the disease is an inflammatory disease, such as rheumatoid arthritis.

In some embodiments, the disease may be a myelodysplastic syndrome, such as systemic mastocytosis, lymphomas and leukemias and solid tumors.

The invention further provides for the use of the oligomer of the invention for the manufacture of a medicament for the treatment of one or more of the diseases referred to herein.

Suitable dosages, formulations, administration routes, compositions, dosage forms, combinations with other therapeutic agents, pro-drug formulations are also provided in PCT/DK2006/000512—which are hereby incorporated by reference.

The invention also provides for a pharmaceutical composition comprising a compound or a conjugate as herein described or a conjugate, and a pharmaceutically acceptable diluent, carrier or adjuvant. PCT/DK2006/000512 provides suitable and preferred pharmaceutically acceptable diluent, carrier and adjuvants—which are hereby incorporated by reference.

Pharmaceutical compositions comprising more than one active ingredient (i.e. comprise a further therapeutic agent or treatment).

The pharmaceutical composition according to the invention may further comprise other active ingredients, including those which are indicated as being useful for the treatment of hyperproliferative diseases and cancer, such as those referred to herein.

The further active ingredients (also referred to as further therapeutic agents) may for instance be selected from one or more of the following (the references referred to which indicate the benefit of combination of the further active ingredient with Mcl-1 targeting antisense oligomers, are hereby incorporated by reference):

PKC412, AM107 and/or Imatinib (Blood. 2007 Apr. 1; 109(7):3031-41.)
Cyclophosphamide (Clin. Cancer Res. 2004 Jun. 15; 10(12 Pt 1):4185-91)
Docetaxel and/or cisplatin (Cancer Biol. Ther. 2006 October; 5(10):1348-54.)
Dacarbazine (J. Invest Dermatol. 2003 June; 120(6):1081-6)
FLT-3 kinase inhibitor (such as those disclosed in WO 2007/147613, hereby incorporated by reference) including Gleevec™ (Imatinib mesylate).

In some embodiments the further therapeutic agents may be used in a combination therapy where the subject is administered the pharmaceutical composition of the invention and the further therapeutic agent, such as the FLT-3 kinase inhibitor, either in the same composition in as a seperate administration. In some embodiments, such a separate administration may be provided prior to, during or subsequent to the pharmaceutical composition of the invention. Suitably both the oligomer targeting Mcl1 and the further active ingredient are administered in effective amounts. In this respect, it is considered that for some further active ingredients, the down-regulation of Mcl1 is beneficial to the treatment with the further active ingredient and may alleviate a non-responsiveness or low-responsiveness to the further active ingredient.

In some embodiments, the further active ingredients is an alkylating agent, such as Cisplatin.

In some embodiments, the further active ingredients is a histone deacetylase inhibitor, such as Trichostatin A.

In some embodiments, the further active ingredients is a glucocorticoid, such as Dexamethasone.

The invention provides for use of an oligomer targeting Mcl1, such as one or more of the oligomers described herein, for the preparation of a medicament, wherein said medicament is for the use in the treatment of cancer in combination with a further active ingredient, such as a further active ingredient selected from the group consisting an alkylating agent, such as Cisplatin, a histone deacetylase inhibitor, such as Trichostatin A, and a glucocorticoid, such as Dexamethasone.

The invention provides for a medicament comprising an oligomer targeting Mcl1, such as one or more of the oligomers described herein, wherein said medicament is for the use in the treatment of cancer in combination with a further active ingredient, such as a further active ingredient selected from the group consisting an alkylating agent, such as Cisplatin, a histone deacetylase inhibitor, such as Trichostatin A, and a glucocorticoid, such as Dexamethasone.

The invention further relates to methods of treating a disease, such as those referred to herein, such as cancer, comprising administering to a patient in need there of an effective amount of an oligomer that targets Mcl1 mRNA in a cell and an effective amount of a further active ingredient, such as a further active ingredient selected from the group consisting an alkylating agent, such as Cisplatin, a histone deacetylase inhibitor, such as Trichostatin A, and a glucocorticoid, such as Dexamethasone, or a pharmaceutically acceptable derivative thereof.

The further active ingredient is typically administered in the form of a pharmaceutical composition which further comprises a pharmaceutically acceptable diluent, carrier, salt or adjuvant.

### Administration

The oligomer targeting Mcl1 may be administered at regular intervals (Dose intervals, DI) of between 3 days and two weeks, such as 4, 5, 6, 7, 8, 9, 0, 11, 12, 13 days, such as about 1 week, such as 6, 7 or 8 days. Suitably at least two doses are provides with a DI period between the two dosages, such as 3, 4, 5, 6, 7, 8, 9 or 10 dosages, each with a dose interval (DI) between each dose of LNA oligomer. The DI period between each dosage may the same, such as between 3 days and two weeks, such as 4, 5, 6, 7, 8, 9, 10, 11, 12, 13 days, such as about 1 week, such as 6, 7 or 8 days.

In some embodiments, each dose of the oligomer targeting Mcl1 may be between about 0.25 mg/kg-about 10 mg/kg, such as about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg. In some embodiments, each dose of the LNA oligomer targeting Mcl1 may be between about 2 mg/kg-about 8 mg/kg, or about 4 to about 6 mg/kg or about 4 mg/kg to about 5 mg/kg. In some embodiments, each dose of the LNA oligomer targeting Mcl1 is at least 2 mg/kg, such as 2, 3, 4, 5, 6, 7 or 8 mg/kg, such as 6 mg/kg.

Administration of the oligomer is typically performed by parenteral administration, such as subcutaneous, intramuscular, intravenous or intra peritoneal administration. Intravenously administration is preferred.

In some embodiments the dosage regime for the oligomer may be repeated after an initial dosage regime, indeed the dosage regime may be repeated as necessary in order to treat or prevent the progression of the disease.

One advantage of the oligomers targeting Mcl1 according to the invention is that they may be administered over a relatively short time period rather than continuously. This provides a marked improvement in the quality of life for the patient as they are not required to be hospital bound for long periods of time. Therefore in a preferred embodiment, the LNA oligomer targeting Mcl1 is not administered by continuous infusion. Each dose of the oligomer may therefore be administered to the patient in a time period of less than 12 hours, such as less than about 8 hours, less than about 4 hours, such as less than about 3 hours. Each dose of the LNA oligomer may therefore be administered to the patient in a time period of between about 1 and about 4 hours, such as between about 2 and about 3 hours, or about 2 hours. The oligomer may be administered to the patient in a time period of at least 30 minutes such as at least 1 hour. Such administrations may be given intravenously, for example.

A pharmaceutically effective dose of the further active ingredient may, in some embodiments be administered prior to, during or subsequently to the administration of one or more pharmaceutically effective doses of the LNA oligomer targeting Mcl1. Typically, one or more effective doses of the further active ingredient is administered so that the both the LNA oligomer and the further active ingredient provide their therapeutic benefit concurrently within the patient or subject.

Combination with Antisense Oligomers Targeting Bcl-2

In some aspects the further therapeutic agent (active ingredient) may be a compound which targets Bcl-2. The (Mcl-1) oligomers according to the invention may, in some embodiments be used in combination with compounds which target Bcl-2, such as antisense oligomers which are complementary to the Bcl-2 mRNA, such as the Genasense compound referred to as (G3139) which is complementary to the first six codons of the human BCl₂ sequence—(i.e. has a sequence ‘tctccagcgtccgccat’), and other antisense Bcl-2 oligomers, such as those disclosed in WO2005/061710, US2005/0170377, U.S. Pat. No. 6,291,668, WO02/17852, US2005/0032714, WO2004/046327, U.S. Provisional Applications U.S. 61/012,185, and U.S. 61/106,261, which are all hereby incorporated by reference.

In some embodiments, the oligomers which target Bcl2, which may be used in combination with the oligomers which target Mcl1, such as those disclosed herein, are LNA oligomers, such as gapmers and shortmers, which comprises a contiguous nucleotide sequence of a total of between 10-50 nucleotides, wherein said contiguous nucleotide sequence is at least 80%, such as at least homologous to a corresponding region of a nucleic acid which encodes a mammalian Bcl-2.

With respect to the structure of the oligomers, such as gapmers and shortmers, and conjugates thereof, which target Bcl-2, with the exception of the fact that such oligomers will be complementary to a corresponding region of the Bcl-2 mRNA, or in one case, comprise 1, 2, 3, or 4 mismatches to the corresponding region of the target Bcl-2, they may, in some embodiments may have the same structure as the oligomers according to the present invention (i.e. apart from the specific sequence of nucleotides).

The administration of a pharmaceutical composition comprising an oligomer which targets Bcl-2 may be performed as per the oligomers which target Mcl1 as disclosed herein, and may be performed prior to, during or after the administration of the pharmaceutical composition comprising the oligomer of the invention, are as part of the same pharmaceutical composition.

The invention therefore provides for methods for the simultaneous (concurrent) inhibition of expression of both Bcl-2 and Mcl-1 in a cell, such as a cancer cell, which is expressing Bcl-2 and Mcl-1, said method comprising
a. administering an effective amount of a Bcl2 inhibitor, such as a oligomer which targets

Bcl2, such as SEQ ID NO 128 or a conjugate or pharmaceutical composition thereof, to said cell so as to inhibit Bcl-2 in said cell,
b. administering an effective amount of a Mcl1 inhibitor, such as a oligomer which targets Mcl1, such as oligomer according to the invention, or a conjugate or pharmaceutical composition thereof, to said cell so as to inhibit Mcl1 in said cell,

wherein steps a) and b) may be performed in any order or simultaneously and lead to the simultaneous inhibition (down-regulation) of both Mcl-1 and Bcl-2 inhibition in said cell; wherein said method is performed either in vivo or in vitro. Suitably the cell may be a cancer cell in a subject, such as a human subject suffering from cancer. In some embodiments, the method may result in cell death such as apoptosis of the cell.

Suitable Bcl2 inhibitors to be used in conjunction with the Mcl1 inhibitor are referenced above and include also include those disclosed in U.S. Provisional Application Ser. No. U.S. 61/012,185, filed 7 Dec. 2007, and U.S. 61/106,261, filed 17 Oct. 2008, the disclosures of which are both incorporated herein by reference in their entirety.

### Kits of Parts

The invention also provides a kit of parts wherein a first part comprises the oligomer, the conjugate and/or the pharmaceutical composition according to the invention and a further part comprises an further active ingredient (i.e. further therapeutic agent), such as those referred to herein. It is therefore envisaged that the kit of parts may be used in a method of treatment, as referred to herein, where the method comprises administering both the first part and the further part, either simultaneously or one after the other.

The invention also provides a kit of parts wherein a first part comprises the oligomer, the conjugate and/or the pharmaceutical composition according to the invention and a further part comprises an antisense oligonucleotide capable of lowering the expression of Bcl 2. It is therefore envisaged that the kit of parts may be used in a method of treatment, as referred to herein, where the method comprises administering both the first part and the further part, either simultaneously or one after the other.

### Medical Methods and Use

The oligomers, conjugates, and compositions according to the invention can be used for the treatment of cell (hyper) proliferation conditions such as cancer and mastocytosis.

In some embodiments, the disease is a myelodysplastic syndrome, such as systemic mastocytosis, lymphomas and leukemias and solid tumors

The cancer, as referred to herein, is in some embodiments selected from the group consisting of melanoma, leukemia, myeloma, lymphoma, glioma, and carcinoma.

In some embodiments the cancer is selected from the group consisting of leukemia, chronic myeloid leukemia (CML), chronic lymphocytic leukemia (CLL) and acute lymphoid leukemia (ALL), malignant glioma, melanoma, multiple myeloma, and hepatocellular carcinoma.

In some embodiments the cancer selected from the group consisting of leukemia, melanoma, myeloma, and melanoma.

In some embodiments the cancer is leukemia, such as chronic lymphocytic leukemia (CLL), acute lymphoid leukemia (ALL), and chronic myeloid leukemia.

In some embodiments the cancer is lymphoma, such as non-hodgkin's lymphomas, follicular lymphoma and diffuse large B-cell lymphoma. In some embodiments the cancer is myleoma such as multiple myeloma. In some embodiments the cancer is melanoma, such as malignant melanoma. In one embodiment the cancer is malignant glioma. In some embodiments the cancer is a carcinoma such as a hepatocellular carcinoma.

The mastocytosis may, in some embodiments, be systemic mastocytosis.

The oligomers, conjugates, and compositions may, in one preferable embodiment, may be for the use in the treatment of the cancers.

In some embodiments, the cancer is liver or kidney cancer.

In some embodiments, for example for the treatment of brain cancer, it is preferred that phosphorothioate linkages are not used in the compound according to the invention.

The oligomers, conjugates, and compositions may, in one preferable embodiment, may be for the use in the treatment of the cancers.

The oligomers, conjugates, and compositions according to the invention can be used for the treatment of inflammatory diseases, such as rheumatoid arthritis.

In some embodiments, the oligomers, conjugates, and compositions according to the invention can be used for the treatment of inflammatory disorders, such as inflammatory disorders selected from the group consisting of: Acute and chronic inflammatory disorders such as sepsis, rheumatoid arthritis (RA), juvenile idiopathic arthritis, ankylosing spondylitis (Bechterew's disease), inflammatory bowel diseases (Crohn's diseases and ulcerative colitis), severe psoriasis, chronic uveitis, sarcoidosis, Wegener's granulomatosis, and other diseases with inflammation as a central feature.

It will be recognised that as Mcl-1 is indicated in inflammation, anti-Mcl-1 oligomers, such as the oligomers referred to herein, may also be used in the treatment of infectious diseases, particularly infectious diseases where an inflammation is a feature.

The invention further provides use of a compound of the invention in the manufacture of a medicament for the treatment of a disease, disorder or condition as referred to herein.

Generally stated, one aspect of the invention is directed to a method of treating a mammal suffering from or susceptible to diseases, disorders or conditions disclosed herein, or those associated with expression or abnormal levels of Mcl-1, comprising administering to the mammal and therapeutically effective amount of an oligomer targeted to Mcl-1 that comprises one or more LNA units.

An interesting aspect of the invention is directed to the use of an oligomer (compound) as defined herein or as conjugate as defined herein for the preparation of a medicament for the treatment of a disease, disorder or condition as referred to herein.

The methods of the invention are preferably employed for treatment or prophylaxis against diseases caused by abnormal levels of Mcl-1.

Furthermore, the invention described herein encompasses a method of preventing or treating a disease comprising a therapeutically effective amount of a Mcl-1 modulating oligomer to a human in need of such therapy. The invention further encompasses the use of a short period of administration of a Mcl-1 modulating oligonucleotide compound. Such uses and methods may further comprise the administration of a further therapeutic compound such as those referred to herein.

The oligomers of the invention may also be conjugated to active drug substances, for example, aspirin, ibuprofen, a sulfa drug, an antidiabetic, an antibacterial or an antibiotic.

Alternatively stated, the invention is furthermore directed to a method for treating abnormal levels of Mcl-1, said method comprising administering a oligomer of the invention, or a conjugate of the invention or a pharmaceutical composition of the invention to a patient in need thereof and further comprising the administration of a further chemotherapeutic agent. Said further administration may be such that the further chemotherapeutic agent is conjugated to the compound of the invention, is present in the pharmaceutical composition, or is administered in a separate formulation.

The invention also relates to an oligomer, a composition or a conjugate as defined herein for use as a medicament.

The invention further relates to use of a compound, composition, or a conjugate as defined herein for the manufacture of a medicament for the treatment of a disease or disorders as referred to herein, such as diseases or disorders associated with abnormal levels of Mcl-1. Such treatment may also comprise the administration (or use) of a further therapeutic agent, as referred to herein, for example an FLT-3 kinase inhibitor.

In some embodiments, said abnormal levels of Mcl-1 is in the form of, or causes, or is characterised by a disease or disorder as referred to herein, such as hyper cell proliferation, such as cancer, such as the cancers referred to herein.

Moreover, the invention relates to a method of treating a subject suffering from a disease or disorder or condition referred to herein, such as a disease or disorder selected from cell (hyper)proliferation disorders, such as cancers, the cancers referred to herein, the method comprising the step of administering a pharmaceutical composition as defined herein to the subject in need thereof. Such methods may further comprise the administration of a further therapeutic agent, as referred to herein, for example an FLT-3 kinase inhibitor or a Bcl1 antisense oligomer.

A patient who is in need of treatment is a patient suffering from or likely to suffer from the disease or disorder).

### Embodiments

The following embodiments of the present invention may be used in combination with the other embodiments described herein.

1. An oligomer of between 10-50 nucleotides in length which comprises a contiguous nucleotide sequence of a total of between 10-50 nucleotides, wherein said contiguous nucleotide sequence is at least 80% homologous to a corresponding region of a nucleic acid which encodes a mammalian Mcl-1, wherein said contiguous nucleotide sequence is present in a nucleic acid sequence selected from the group consisting of SEQ ID NOs 95-117 and/or 2-18 and/or 77-82, wherein optionally, said oligomer comprises at least one LNA unit.
2. The oligomers according to embodiment 1, wherein said contiguous nucleotide sequence is present in a nucleic acid sequence selected from the group consisting of SEQ ID NOs 100, 101, 104, 108, 110, 111 and 111, or 7, 8, 11, 15, 16, 17 and 18.
3. The oligomers according to embodiment 1, wherein said contiguous nucleotide sequence is present in a nucleic acid sequence selected from the group consisting of SEQ ID NOs 95, 96, 97, 98, 99, 100, 101, 102, 103, 105, 106, and 107 (and/or 113, 114 and 11%), or SEQ ID NO 2, 3, 4, 5, 6, 7, 8, 9, 10, 12, 13, and 14 (and/or 78, 79 and 80), wherein said oligomer comprises at least one LNA unit.
4. The oligomer according to any one of embodiments 1-3, wherein the contiguous nucleotide sequence comprises no more than 3, such as no more than 2 mismatches to the corresponding region of a nucleic acid which encodes a mammalian Mcl-1.
5. The oligomer according to embodiment 4, wherein said contiguous nucleotide sequence comprises no more than a single mismatch to the corresponding region of a nucleic acid which encodes a mammalian Mcl-1.
6. The oligomer according to embodiment 5, wherein said contiguous nucleotide sequence comprises no mismatches, (i.e. is complementary to) the corresponding region of a nucleic acid which encodes a mammalian Mcl-1.
7. The oligomer according to any one of embodiments 1-6, wherein the nucleotide sequence of the oligomer consists of the contiguous nucleotide sequence.
8. The oligomer according to any one of embodiments 1-7, wherein the nucleic acid which encodes a mammalian Mcl-1 is the human Mcl-1 nucleotide sequence such as SEQ ID No 1, or a naturally occurring allelic variant thereof.
9. The oligomer according to any one of embodiments 1-8, wherein the contiguous nucleotide sequence is complementary to a corresponding region of both the human Mcl-1 nucleic acid sequence and a non-human mammalian Mcl-1 nucleic acid sequence, such as the mouse or monkey Mcl-1 nucleic acid sequence.
10. The oligomer according to any one of embodiments 1 to 9, wherein the contiguous nucleotide sequence comprises a contiguous subsequence of at least 7, nucleotide residues which, when formed in a duplex with the complementary Mcl-1 target RNA is capable of recruiting RNaseH.
11. The oligomer according to embodiment 10, wherein the contiguous nucleotide sequence comprises of a contiguous subsequence of at least 8, at least 9 or at least 10 nucleotide residues which, when formed in a duplex with the complementary Mcl-1 target RNA is capable of recruiting RNaseH.
12. The oligomer according to any one of embodiments 10 or 11 wherein said contiguous subsequence is at least 9 or at least 10 nucleotides in length, such as at least 12 nucleotides or at least 14 nucleotides in length, such as 14, 15 or 16 nucleotides residues which, when formed in a duplex with the complementary Mcl-1 target RNA is capable of recruiting RNaseH.
13. The oligomer according to embodiment any one of embodiments 1-12 wherein said oligomer is conjugated with one or more non-nucleotide compounds.
14. The oligomer according to any one of embodiments 1-13, wherein said oligomer has a length of between 10-22 nucleotides.
15. The oligomer according to any one of embodiments 1-13, wherein said oligomer has a length of between 12-18 nucleotides.
16. The oligomer according to any one of embodiments 1-13, wherein said oligomer has a length of 14, 15 or 16 nucleotides.
17. The oligomer according to any one of embodiments 1-16, wherein said continuous nucleotide sequence corresponds to a contiguous nucleotide sequence present in a nucleic acid sequence selected from the group consisting of SEQ ID NO 36, 42, 47, 50, 55, 58, 61, 64, or 66.
18. The oligomer according to any one of embodiments 1-17, wherein the oligomer or contiguous nucleotide sequence is selected from the group consisting of SEQ ID NO 36-69, or wherein the oligomer or contiguous nucleotide sequence consists or comprises of an equivalent nucleotide sequence to the nucleotide sequence selected from SEQ ID NO 36-69,
19. The oligomer according to any some embodiments 1-18 wherein said oligomer is single stranded.
20. The oligomer according to any one of embodiments 1-19, wherein said contiguous nucleotide sequence comprises at least one affinity enhancing nucleotide analogue.
21. The oligomer according to embodiment 20, wherein said contiguous nucleotide sequence comprises a total of 2, 3, 4, 5, 6, 7, 8, 9 or 10 affinity enhancing nucleotide analogues, such as LNA, such as between 5 and 8 affinity enhancing nucleotide analogues.
22. The oligomer according to any one of embodiments 1-21 which comprises at least one affinity enhancing nucleotide analogue, wherein the remaining nucleotides are selected from the group consisting of DNA nucleotides and RNA nucleotides, preferably DNA nucleotides.
23. The oligomer according to any one of embodiments 1-22, wherein the oligomer comprises of a sequence of nucleotides of formula, in 5′ to 3′ direction, A-B-C, and optionally of formula A-B-C-D, wherein:
A consists or comprises of at least one nucleotide analogue, such as 1, 2, 3, 4, 5 or 6 nucleotide analogues, preferably between 2-5 nucleotide analogues, preferably 2, 3 or 4 nucleotide analogues, most preferably 2, 3 or 4 consecutive nucleotide analogues and;
B consists or comprises at least five consecutive nucleotides which are capable of recruiting RNAseH (when formed in a duplex with a complementary RNA molecule, such as the Mcl-1 mRNA target), such as DNA nucleotides, such as 5, 6, 7, 8, 9, 10, 11 or 12 consecutive nucleotides which are capable of recruiting RNAseH, or between 6-10, or between 7-9, such as 8 consecutive nucleotides which are capable of recruiting RNAseH, and;
C consists or comprises of at least one nucleotide analogue, such as 1, 2, 3, 4, 5, or 6 nucleotide analogues, preferably between 2-5 nucleotide analogues, such as 2, 3 or 4 nucleotide analogues, most preferably 2, 3 or 4 consecutive nucleotide analogues, and;
D when present, consists or comprises, preferably consists, of one or more DNA nucleotide, such as between 1-3 or 1-2 DNA nucleotides.
24. The oligomer according to embodiment 23, wherein region A consists or comprises of 2, 3 or 4 consecutive nucleotide analogues.
25. The oligomer according to any one of embodiments 23-24, wherein region B consists or comprises of 7, 8, 9 or 10 consecutive DNA nucleotides or equivalent nucleotides which are capable of recruiting RNAseH when formed in a duplex with a complementary RNA, such as the Mcl-1 mRNA target.
26. The oligomer according to any one of embodiments 23-25, wherein region C consists or comprises of 2, 3 or 4 consecutive nucleotide analogues.
27. The oligomer according to any one of embodiments 23-26, wherein region D consists, where present, of one or two DNA nucleotides.
28. The oligomer according to any one of embodiments 23-27, wherein:
A Consists or comprises of 3 contiguous nucleotide analogues;
B Consists or comprises of 7, 8, 9 or 10 contiguous DNA nucleotides or equivalent nucleotides which are capable of recruiting RNAseH when formed in a duplex with a complementary RNA, such as the Mcl-1 mRNA target;
C Consists or comprises of 3 contiguous nucleotide analogues;
D Consists, where present, of one or two DNA nucleotides.
29. The oligomer according to embodiment 23, wherein the contiguous nucleotide sequence consists of 10, 11, 12, 13 or 14 nucleotides, and wherein;
A Consists of 1, 2 or 3 contiguous nucleotide analogues;
B Consists of 7, 8, or 9 consecutive DNA nucleotides or equivalent nucleotides which are capable of recruiting RNAseH when formed in a duplex with a complementary RNA, such as the Mcl-1 mRNA target;
C Consists of 1, 2 or 3 contiguous nucleotide analogues;
D Consists, where present, of one DNA nucleotide.
30. The oligomer according to anyone of embodiments 23-28, wherein B comprises at least one LNA nucleotide which is in the alpha-L configuration, such as alpha-L-oxy LNA.
31. The oligomer according to any one of embodiments 1-30, wherein the nucleotide analogue(s) are independently or collectively selected from the group consisting of: Locked Nucleic Acid (LNA) units; 2′-O-alkyl-RNA units, 2′-OMe-RNA units, 2′-amino-DNA units, 2′-fluoro-DNA units, PNA units, HNA units, and INA units.
32. The oligomer according to embodiment 31 wherein all the nucleotide analogues(s) are LNA units.
33. The oligomer according to any one of embodiments 1-32, which comprises 1, 2, 3, 4, 5, 6, 7. 8. 9 or 10 LNA units such as between 2 and 8 nucleotide LNA units.
34. The oligomer according to any one of the embodiments 30-32, wherein the LNAs are independently selected from oxy-LNA, thio-LNA, and amino-LNA, in either of the beta-D and alpha-L configurations or combinations thereof.
35. The oligomer according to embodiment 33, wherein the LNAs are all beta-D-oxy-LNA.
36. The oligomer according to any one of embodiments 22-34, wherein the nucleotide analogues or nucleotides of regions A and C are beta-D-oxy-LNA.
37. The oligomer according to any one of embodiments 1-36, wherein at least one of the nucleotides present in the oligomeris a modified nucleotide selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.
38. The oligomer according to any one of embodiments 1-37, wherein said oligomer hybridises with a corresponding mammalian Mcl-1 mRNA with a Tₘ of at least 40° C. of 50° C.
39. The oligomer according to any one of embodiments 1-38, wherein said oligomer hybridises with a corresponding mammalian Mcl-1 mRNA with a Tₘ of no greater than 80° C.
40. The oligomer according to any one of embodiments 1-39, wherein the internucleoside linkages are independently selected from the group consisting of: phosphodiester, phosphorothioate and boranophosphate.
41. The oligomer according to embodiment 40, wherein the oligomer comprises at least one phosphorothioate internucleoside linkage.
42. The oligomer according to embodiment 41, wherein the internucleoside linkages adjacent to or between DNA or RNA units, or within region B are phosphorothioate linkages.
43. The oligomer according to embodiment 40 or 41, wherein the linkages between at least one pair of consecutive nucleotide analogues is a phosphodiester linkage.
44. The oligomer according to embodiment 40 or 41, wherein all the linkages between consecutive nucleotide analogues are phosphodiester linkages.
45. The oligomer according to embodiment 40 wherein all the internucleoside linkages are phosphorothioate linkages.
46. A conjugate comprising the oligomer according to any one of the embodiments 1-45 and at least one non-nucleotide or non-polynucleotide moiety covalently attached to said compound.
47. A pharmaceutical composition comprising an oligomer as defined in any of embodiments 1-45 or a conjugate as defined in embodiment 46, and a pharmaceutically acceptable diluent, carrier, salt or adjuvant.
48. A pharmaceutical composition according to 47, wherein the oligomer is constituted as a pro-drug.
49. A pharmaceutical composition according to any one of embodiments 46-48, which further comprises a further therapeutic agent such as an active ingredient which targets Bcl-2 such as antisense oligomers which target Bcl-2 mRNA.
50. A pharmaceutical composition according to any one of embodiments 46-49, which further comprises a FLT-3 kinase inhibitor.
51. Use of an oligomer as defined in any one of the embodiments 1-45, or a conjugate as defined in embodiment 46, for the manufacture of a medicament for the treatment of a disease or disorder selected from the group consisting of hyperproliferative disorder such as cancer and mastocytosis; and inflammatory diseases, such as rheumatoid arthritis.
52. A method for treating an disease or disorder selected from the group consisting of hyperproliferative disorder such as cancer and mastocytosis, and inflammatory diseases, said method comprising administering an oligomer as defined in one of the embodiments 1-45, or a conjugate as defined in embodiment 46, or a pharmaceutical composition as defined in any one of the embodiments 47-50, to a patient in need thereof.
53. A method of reducing or inhibiting the expression of Mcl-1 in a cell or a tissue, the method comprising the step of contacting said cell or tissue with an oligomer as defined in one of the embodiments 1-45, or a conjugate as defined in embodiment 46, or a pharmaceutical composition as defined in any one of the embodiments 47-50, so that expression of Mcl-1 is reduce or inhibited.
54. A method of triggering apoptosis in a cell, such as a cancer cell, said method comprising the step of contacting said cell or tissue with an oligomer as defined in one of the embodiments 1-45, or a conjugate as defined in embodiment 46, or a pharmaceutical composition as defined in any one of the embodiments 47-50, so that so that either expression of Mcl-1 is inhibited or reduced and/or apoptosis is triggered.

### EXAMPLES

### Example 1

### Monomer Synthesis

The LNA monomer building blocks and derivatives were prepared following published procedures and references cited therein—see WO07/031,081 and the references cited therein.

### Example 2

### Oligonucleotide Synthesis

Oligonucleotides were synthesized according to the method described in WO07/031,081. Table 1 shows examples of antisense oligonucleotide sequences of the invention. Tables 2-4 show examples of antisense oligonucleotides (oligos) of the invention.

### Example 3

### Design of the Oligonucleotides

In accordance with the present invention, a series of different oligonucleotides were designed to target different regions of human Mcl-1 (GenBank accession number NM_{—}021960 and NM_{—}182763, SEQ ID NO: 70 and 1).

TABLE 1
Some Exemplary Oligomer Motif sequences of the invention

| | | | Target |
|---|---|---|---|
| SEQ ID NO | Sequence (5′-3′) | Length (bases) | site (NM_182763) |
| SEQ ID NO: 2 | AAGCCAGCAGCACATT | 16 | 870-885 |
| SEQ ID NO: 3 | CTACTCCAGCAACACC | 16 | 891-906 |
| SEQ ID NO: 4 | AAGGCTATCTTATTAG | 16 | 924-939 |
| SEQ ID NO: 5 | CTATCTTATTAGATAT | 16 | 920-935 |
| SEQ ID NO: 6 | TCTTATTAGATATGCC | 16 | 917-932 |
| SEQ ID NO: 7 | TTCCCATGTATTTATT | 16 | 1087-1102 |
| SEQ ID NO: 8 | ATGTATTTATTCTTGT | 16 | 1082-1097 |
| SEQ ID NO: 9 | TTTATTCTTGTTAGCC | 16 | 1077-1092 |
| SEQ ID NO: 10 | AAACCAAGCCAAAGTA | 16 | 1489-1504 |
| SEQ ID NO: 11 | ACTAGGCTAATAAA | 14 | 1518-1531 |
| SEQ ID NO: 12 | AAATAGACTTTCTGTA | 16 | 1640-1655 |
| SEQ ID NO: 13 | AATAGCACCATGGTT | 15 | 2263-2277 |
| SEQ ID NO: 14 | TTTCAAATGACCCTAG | 16 | 2939-2954 |
| SEQ ID NO: 15 | AACTACATAAAGTGCT | 16 | 3164-3179 |
| SEQ ID NO: 16 | GTAAGACAAACAGA | 14 | 3532-3545 |
| SEQ ID NO: 17 | GAGATAATCTCCAGC | 15 | 664-678 |
| SEQ ID NO: 18 | GACTCCTTACTGGA | 14 | 71-84 |
| SEQ ID NO: 77 | GCTTCTTTCAGACAGT | 16 | 1129-1144 |
| SEQ ID NO: 78 | TTTCAGACAGTGACTC | 16 | 1124-1139 |
| SEQ ID NO: 79 | TTCAGACAGTGACTCT | 16 | 1123-1138 |
| SEQ ID NO: 80 | TTACATTCTTAGTCAT | 16 | 3017-3032 |
| SEQ ID NO: 81 | AAAGTCCTGAACACTT | 16 | 1462-1477 |
| SEQ ID NO: 82 | CAGCTCCTACTCCAGC | 16 | 897-912 |
| SEQ ID NO: 95 | GCAAAAGCCAGCAGCACATTCCTG | 24 | 866-889 |
| SEQ ID NO: 96 | GCTCCTACTCCAGCAACACCTGCA | 24 | 887-910 |
| SEQ ID NO: 97 | CAGTAAGGCTATCTTATTAGATAT | 24 | 920-943 |
| SEQ ID NO: 98 | AAGGCTATCTTATTAGATATGCCA | 24 | 916-939 |
| SEQ ID NO: 99 | GCTATCTTATTAGATATGCCAAAC | 24 | 913-936 |
| SEQ ID NO: 100 | ACTCTTCCCATGTATTTATTCTTG | 24 | 1083-1106 |
| SEQ ID NO: 101 | TCCCATGTATTTATTCTTGTTAGC | 24 | 1078-1101 |
| SEQ ID NO: 102 | TGTATTTATTCTTGTTAGCCATAA | 24 | 1073-1096 |
| SEQ ID NO: 103 | ATGGAAACCAAGCCAAAGTATAAC | 24 | 1485-1508 |
| SEQ ID NO: 104 | GATAAACTAGGCTAATAAAGTAAG | 24 | 1513-1536 |
| SEQ ID NO: 105 | AAAGAAATAGACTTTCTGTAAAAA | 24 | 1636-1659 |
| SEQ ID NO: 106 | TAATAATAGCACCATGGTTAGACT | 24 | 2258-2281 |
| SEQ ID NO: 107 | AAGCTTTCAAATGACCCTAGTTCC | 24 | 2935-2958 |
| SEQ ID NO: 108 | TAAAAACTACATAAAGTGCTTTTA | 24 | 3160-3183 |
| SEQ ID NO: 109 | GAAGCGTAAGACAAACAGAAGTCA | 24 | 3527-3550 |
| SEQ ID NO: 110 | ACCGAGAGATAATCTCCAGCGACT | 24 | 660-683 |
| SEQ ID NO: 111 | ACCCCGACTCCTTACTGGAAGGAA | 24 | 66-89 |
| SEQ ID NO: 112 | CTTTGCTTCTTTCAGACAGTGACT | 24 | 1125-1148 |
| SEQ ID NO: 113 | CTTCTTTCAGACAGTGACTCTTCA | 24 | 1120-1143 |
| SEQ ID NO: 114 | TTCTTTCAGACAGTGACTCTTCAA | 24 | 1119-1142 |
| SEQ ID NO: 115 | CCCATTACATTCTTAGTCATCTTA | 24 | 3013-3036 |
| SEQ ID NO: 116 | TATAAAAGTCCTGAACACTTGGAC | 24 | 1462-1481 |
| SEQ ID NO: 117 | AAACCAGCTCCTACTCCAGCAACA | 24 | 893-916 |

TABLE 2
Exemplary Oligonucleotide
designs of the invention

| SEQ ID NO | Sequence (5′-3′) |
|---|---|
| SEQ ID NO: 19 | AsAsGscₛcₛaₛgₛcₛaₛgₛcₛaₛcₛAsTsT |
| SEQ ID NO: 20 | CsTsAscₛtₛcₛcₛaₛgₛcₛaₛaₛcₛAsCsC |
| SEQ ID NO: 21 | AsAsGsgₛcₛtₛaₛtₛcₛtₛtₛaₛtₛTsAsG |
| SEQ ID NO: 22 | CsTsAstₛcₛtₛtₛaₛtₛtₛaₛgₛaₛTsAsT |
| SEQ ID NO: 23 | TsCsTstₛaₛtₛtₛaₛgₛaₛtₛaₛtₛGsCsC |
| SEQ ID NO: 24 | TsTsCscₛcₛaₛtₛgₛtₛaₛtₛtₛtₛAsTsT |
| SEQ ID NO: 25 | AsTsGstₛaₛtₛtₛtₛaₛtₛtₛcₛtsTsGsT |
| SEQ ID NO: 26 | TsTsTsaₛtₛtₛcₛtₛtₛgₛtₛtₛaₛGsCsC |
| SEQ ID NO: 27 | AsAsAscₛcₛaₛaₛgₛcₛcₛaₛaₛaₛGsTsA |
| SEQ ID NO: 28 | AsCsTsaₛgₛgₛcₛtₛaₛaₛtₛAsAsA |
| SEQ ID NO: 29 | AsAsAstₛaₛgₛaₛcₛtₛtₛtₛcₛtₛGsTsA |
| SEQ ID NO: 30 | AsAsTsaₛgₛcₛaₛcₛcₛaₛtₛgₛGsTsT |
| SEQ ID NO: 31 | TsTsTscₛaₛaₛaₛtₛgₛaₛcₛcₛcₛTsAsG |
| SEQ ID NO: 32 | AsAsCstₛaₛcsaₛtₛaₛaₛaₛgₛtₛGsCsT |
| SEQ ID NO: 33 | GsTsAsaₛgₛaₛcₛaₛaₛaₛcₛAsGsA |
| SEQ ID NO: 34 | GsAsGsaₛtₛaₛaₛtₛcₛtₛcₛcₛAsGsC |
| SEQ ID NO: 35 | GsAsCstₛcscₛtₛtₛaₛcₛtₛGsGsA |
| SEQ ID NO: 83 | GsCsTstₛcₛtₛtₛtₛcₛaₛgₛaₛcₛAsGsT |
| SEQ ID NO: 84 | TsTsTscₛaₛgₛaₛcₛaₛgₛtₛgₛaₛCsTsC |
| SEQ ID NO: 85 | TsTsCsaₛgₛaₛcₛaₛgₛtₛgₛaₛcₛTsCsT |
| SEQ ID NO: 86 | TsTsAscₛaₛtₛtₛcₛtₛtₛaₛgₛtₛCsAsT |
| SEQ ID NO: 87 | AsAsAsgₛtₛcₛcₛtₛgₛaₛaₛcₛaₛCsTsT |
| SEQ ID NO: 88 | CsAsGscₛtₛcₛcₛtₛₐₛcₛtₛcₛcₛAsGsC |

In SEQ ID NOs: 19-35, upper case letters indicates nucleotide analogue units, such as LNA units, and subscript “s” represents phosphorothiote linkage.
Lower case letters represent nucleotide units. Absence of “s” (if any) indicates phosphodiester linkage.

### Example 4

### In Vitro Model: Cell Culture

The effect of antisense oligonucleotides on target nucleic acid expression can be tested in any of a variety of cell types provided that the target nucleic acid is present at measurable levels. The target can be expressed endogenously, by natural uptake into cells, or by transient or stable transfection of a nucleic acid encoding said target. The expression level of target nucleic acid can be routinely determined using, for example, Northern blot analysis, Real-Time PCR, Ribonuclease protection assays. The following cell types are provided for illustrative purposes, but other cell types can be routinely used, provided that the target is expressed in the cell type chosen.

Cells were cultured in the appropriate medium as described below and maintained at 37° C. at 95-98% humidity and 5% CO₂. Cells were routinely passaged 2-3 times weekly.

15PC3: The human prostate cancer cell line 15PC3 was cultured in DMEM (Sigma)+10% fetal bovine serum (FBS)+2 mM Glutamax I+gentamicin (25 μg/ml).
Namelwa: The human Burkitt's lymphoma cancer cell line Namalwa was cultured in RPMI 1640 with glutamax (Sigma)+10 mM HEPES+1 mM sodium pyrovate+7.5% FBS+gentamicin (25 μg/ml).
K562: The human chronic myelogenous leukaemia cell line was cultured in RPMI 1640 with glutamax (Sigma)+10% FBS+gentamicin (25 μg/ml).
HepG2: The human hepatocyte carcinoma cell line was cultured in Eagle's Minimum Essential Medium (Sigma)+10% FBS+gentamicin (25 μg/ml).

### Example 5

### In Vitro Model: Treatment with Antisense Oligonucleotide

LipofectAMINE Transfection: The cell lines listed in example 4 were treated with oligonucleotide using the cationic liposome formulation LipofectAMINE 2000 (Gibco) as transfection vehicle. Cells were seeded in 6-well cell culture plates (NUNC) and treated when 80-90% confluent. Oligo concentrations used ranged from 1 nM to 25 nM final concentration. Formulation of oligo-lipid complexes were carried out essentially as described by the manufacturer using serum-free OptiMEM (Gibco) and a final lipid concentration of 5 μg/ml LipofectAMINE 2000. Cells were incubated at 37° C. for 4 hours and treatment was stopped by removal of oligo-containing culture medium. Cells were washed and serum-containing media was added. After oligo treatment cells were allowed to recover for 20 hours before they were harvested for RNA analysis.

Natural Uptake: The cell lines 15PC3, K562 and Namalwa listed in example 4 were incubated with oligo dissolved in sterile water without any transfection vehicle. Cells were seeded in 6-well cell culture plates (NUNC) and incubated with oligo when 10-30% confluent. Oligo concentrations used ranged from 1 μM to 10 μM, final concentration. Cells were incubated at 37° C. in the oligo containing normal growth serum for 1 to 15 days before they were harvested for RNA analysis

### Example 6

### In Vitro Model: Extraction of RNA and cDNA Synthesis

### Total RNA Isolation and First Strand Synthesis

Total RNA was extracted from cells transfected as described above and using the Qiagen

RNeasy kit (Qiagen cat. no. 74104) according to the manufacturer's instructions. First strand synthesis was performed using Reverse Transcriptase reagents from Ambion according to the manufacturer's instructions.

For each sample 0.5 μg total RNA was adjusted to (10.8 μl) with RNase free H₂O and mixed with 2 μl random decamers (50 μM) and 4 μl dNTP mix (2.5 mM each dNTP) and heated to 70° C. for 3 min after which the samples were rapidly cooled on ice. After cooling the samples on ice, 2 μl 10× Buffer RT, 1 μl MMLV Reverse Transcriptase (100 U/μl) and 0.25 μl RNase inhibitor (10 U/μl) was added to each sample, followed by incubation at 42° C. for 60 min, heat inactivation of the enzyme at 95° C. for 10 min and then the sample was cooled to 4° C.

### Example 7

### In Vitro Model: Analysis of Oligonucleotide Inhibition of Mcl-1 Expression by Real-time PCR

Antisense modulation of Mcl-1 expression can be assayed in a variety of ways known in the art. For example, Mcl-1 mRNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA.

Methods of RNA isolation and RNA analysis such as Northern blot analysis is routine in the art and is taught in, for example, Current Protocols in Molecular Biology, John Wiley and Sons.

Real-time quantitative (PCR) can be conveniently accomplished using the commercially available Multi-Color Real Time PCR Detection System, available from Applied Biosystem.

Real-time Quantitative PCR Analysis of Mcl-1 mRNA Levels: The sample content of human Mcl-1 mRNA was quantified using the human Mcl-1 ABI Prism Pre-Developed TaqMan Assay Reagents (Applied Biosystems cat. no. Hs00766187_ml according to the manufacturer's instructions.

Glyceraldehyde-3-phosphate dehydrogenase (GAPDH) mRNA quantity or beta actin (ACTB) mRNA quantity was used as an endogenous control for normalizing any variance in sample preparation.

The sample content of human GAPDH mRNA was quantified using the human GAPDH ABI Prism Pre-Developed TaqMan Assay Reagent (Applied Biosystems cat. no. 4310884E) according to the manufacturer's instructions.

The sample content of human beta actin mRNA was quantified using the human ACTB ABI Prism Pre-Developed TaqMan Assay Reagent (Applied Biosystems cat. no. 4310681E) according to the manufacturer's instructions.

The sample content of murine Mcl-1 mRNA was quantified using the murine Mcl-1 ABI Prism Pre-Developed TaqMan Assay Reagents (Applied Biosystems cat. no. Mm00725832_s1) according to the manufacturer's instructions.

The sample content of murine b-actin mRNA was quantified using the ACTB Mcl-1 ABI Prism Pre-Developed TaqMan Assay Reagents (Applied Biosystems cat. no. 4352341 E) according to the manufacturer's instructions

The sample content of murine GAPDH mRNA was quantified using the murine GAPDH ABI Prism Pre-Developed TaqMan Assay Reagents (Applied Biosystems cat. no. 435239E) according to the manufacturer's instructions

The cDNA from the first strand synthesis performed as described in Example 6 was diluted 2-20 times, and analyzed by real time quantitative PCR using Taqman 7500 FAST or 7900 FAST from Applied Biosystems. The primers and probe were mixed with 2× Taqman Fast Universal PCR master mix (2×) (Applied Biosystems Cat.#436-4103) and added to 4 μl cDNA to a final volume of 10 μl. Each sample was analysed in duplicate. Assaying 2 fold dilutions of a cDNA that had been prepared on material purified from a cell line expressing the RNA of interest generated standard curves for the assays. Sterile H₂O was used instead of cDNA for the no template control. PCR program: 60° C. for 2 minutes, then 95° C. for 30 seconds, followed by 40 cycles of 95° C., 3 seconds, 60° C., 20-30 seconds. Relative quantities of target mRNA sequence were determined from the calculated Threshold cycle using the Applied Biosystems Fast System SDS Software Version 1.3.1.21. or SDS Software Version 2.3.

### Example 8

### In Vitro Analysis: Antisense Inhibition of Human Mcl-1 Expression by Oligonucleotide Compounds

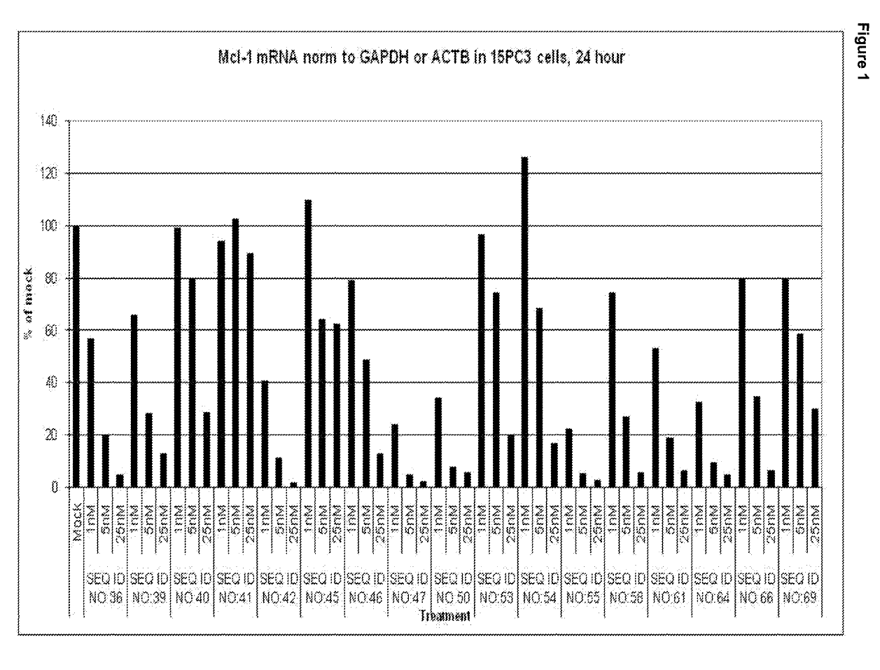

Oligonucleotides presented in Table 3 were evaluated for their potential to knockdown of Mcl-1 at concentrations of 1, 5 and 25 nM (see FIG. 1).

TABLE 3
Antisense Inhibition of Human
Mcl-1 expression by oligonucleotides.

| | | Percent inhibition |
|---|---|---|
| Test | | of Mcl-1 in 15PC3 |
| substance | Sequence (5′-3′) | cells |
| SEQ ID NO: 36 | AsAsGscₛcₛaₛgₛcₛaₛgₛcₛaₛcₛAsTsT | 95 |
| SEQ ID NO: 37 | AsGsCₛcₛaₛgₛcₛaₛgₛcₛaₛcₛAsT | n.d. |
| SEQ ID NO: 38 | GsCₛcₛaₛgₛcₛaₛgₛcₛaₛCₛA | n.d. |
| SEQ ID NO: 39 | CsTsAscₛtₛcₛcₛaₛgₛcₛaₛaₛcₛAsCsC | 87 |
| SEQ ID NO: 40 | AsAsGsgₛcₛtₛaₛtₛcₛtₛtₛaₛtₛTsAsG | 71 |
| SEQ ID NO: 41 | CsTsAstₛcₛtₛtₛaₛtₛtₛaₛgₛaₛTsAsT | 11 |
| SEQ ID NO: 42 | TsCsTstₛaₛtₛtₛaₛgₛaₛtₛaₛtₛGsCsC | 98 |
| SEQ ID NO: 43 | CsTsTₛaₛtₛtₛaₛgₛaₛtₛaₛtₛGsC | n.d. |
| SEQ ID NO: 44 | TsTₛaₛtₛtₛaₛgₛaₛtₛaₛTₛG | n.d. |
| SEQ ID NO: 45 | TsTsCscₛcₛaₛtₛgₛtₛaₛtₛtₛtₛAsTsT | 38 |
| SEQ ID NO: 46 | AsTsGstₛaₛtₛtₛtₛaₛtₛtₛcₛtₛTsGsT | 87 |
| SEQ ID NO: 47 | TsTsTsaₛtₛtₛcₛtₛtₛgₛtₛtₛaₛGsCsC | 98 |
| SEQ ID NO: 48 | TsTsAₛtₛtₛcₛtₛtₛgₛtₛtₛaₛGsC | n.d. |
| SEQ ID NO: 49 | TsAₛtₛtₛcₛtₛtₛgₛtₛtₛAₛG | n.d. |
| SEQ ID NO: 50 | AsAsAₛcₛcₛaₛaₛgₛcₛcₛaₛaₛaₛGsTsA | 94 |
| SEQ ID NO: 51 | AsAsCscₛaₛaₛgₛcₛcₛaₛaₛaₛGsT | n.d. |
| SEQ ID NO: 52 | AsCₛcₛaₛaₛgₛcₛcₛaₛaₛAₛG | n.d. |
| SEQ ID NO: 53 | AsCsTsaₛgₛgₛcₛtₛaₛaₛtₛAsAsAs | 81 |
| SEQ ID NO: 54 | AsAsAstₛaₛgₛaₛcₛtₛtₛtₛcₛtₛGsTsA | 83 |
| SEQ ID NO: 55 | AsAsTsaₛgₛcₛaₛcₛcₛaₛtₛgₛGsTsT | 97 |
| SEQ ID NO: 56 | AsTsAₛgₛcₛaₛcₛcₛaₛtₛgₛGsT | n.d. |
| SEQ ID NO: 57 | TsAₛgₛcₛaₛcₛcₛaₛtₛGₛG | n.d. |
| SEQ ID NO: 58 | TsTsTscₛaₛaₛaₛtₛgₛaₛcₛcₛcₛTsAsG | 94 |
| SEQ ID NO: 59 | TsTsCₛaₛaₛaₛtₛgₛaₛcₛcₛcₛTsA | n.d. |
| SEQ ID NO: 60 | TsCₛaₛaₛaₛtₛgₛaₛcₛcₛCₛT | n.d. |
| SEQ ID NO: 61 | AsAsCstₛaₛcₛaₛtₛaₛaₛaₛgₛtₛGsCsT | 94 |
| SEQ ID NO: 62 | AsCsTsaₛcₛaₛtₛaₛaₛaₛgₛtₛGsC | n.d. |
| SEQ ID NO: 63 | CsTₛaₛcₛaₛtₛaₛaₛaₛgₛTₛG | n.d. |
| SEQ ID NO: 64 | GsTsAsaₛgₛaₛcₛaₛaₛaₛcₛAsGsA | 95 |
| SEQ ID NO: 65 | TsAsaₛgₛaₛcₛaₛaₛaₛcₛAsG | n.d. |
| SEQ ID NO: 66 | GsAsGsaₛtₛaₛaₛtₛcₛtₛcₛcₛAsGsC | 93 |
| SEQ ID NO: 67 | GsAsGsaₛtₛaₛaₛtₛcₛtₛcₛcₛAsG | n.d. |
| SEQ ID NO: 68 | GsAₛtₛaₛaₛtₛcₛtₛcₛcₛAsG | n.d. |
| SEQ ID NO: 69 | GsAsCstₛcₛcₛtₛtₛaₛcₛtₛGsGsA | 70 |

The data in Table 3 are presented as percentage down-regulation relative to mock transfected cells at 25 nM.
Lower case letters represent DNA units, bold upper case letters represent LNA preferably, β-D-oxy-LNA units.
All LNA C are preferably 5′methyl C. Subscript “s” represents phosphorothioate linkage.

As shown in Table 3, oligonucleotides of SEQ ID NOs: 36, 42, 47, 50, 55, 58, 61, 64, and 66 demonstrated about 90% or greater inhibition of Mcl-1 expression at 25 nM in 15PC3 cells in these experiments and are therefore preferred.

Further oligonucleotides based on the illustrated antisense oligo sequences, mat be prepared, for example, by varying the length (shorter or longer) and/or nucleotide content (e.g. the type and/or proportion of analogue units). Further exemplary oligomers according to the invention as provided in the table below (see FIG. 14)

TABLE 4

| SEQ | |
|---|---|
| ID | Sequence |
| 89 | 5′-GsmCsTstₛcₛtₛtₛtₛcₛaₛgₛaₛcₛAsGsT-3′ |
| 90 | 5′-TsTsTscₛaₛgₛaₛcₛaₛgₛtₛgₛaₛmCsTsmC-3′ |
| 91 | 5′-TsTsmCsaₛgₛaₛcₛaₛgₛtₛgₛaₛcₛTsmCsT-3′ |
| 92 | 5′-TsTsAscₛaₛtₛtₛcₛtₛtₛaₛgₛtₛmCsAsT-3′ |
| 93 | 5′-AsAsAsgₛtₛcₛcₛtₛgₛaₛaₛcₛaₛmCsTsT-3′ |
| 94 | 5′-mCsAsGscₛtₛcₛcₛtₛaₛcₛtₛcₛcₛAsGsmC-3′ |

Lower case letters represent DNA units, bold upper case letters represent
LNA preferably, β-D-oxy-LNA units. All LNA C are preferably 5′methyl C.
Subscript “s” represents phosphorothioate linkage.

### Example 9

### In Vitro Analysis: Apoptosis Induction by Lna Antisense Oligomeric Compounds

Some preferred oligos (SEQ ID NOs: 36, 42, 47, 50, 55, 58, 61, 64, and 66) presented in Table 3 were selected and evaluated for their potential to induce apoptosis in 15PC3 cells and HUH-7 cells.

15PC3 cells were seeded to a density of 6,000 cells per well in white 96 well plate (Nunc 136101) in DMEM the day prior to transfection. HUH-7 cells were seeded to a density of 8,500 cells per well in white 96 well plate (Nunc 136101) in DMEM the day prior to transfection. The next day cells were washed once in prewarmed OptiMEM followed by addition of 72 μl OptiMEM containing 5 μg/ml Lipofectamine-2000 (In vitrogen). Cells were incubated for 7 min before adding 18 μl oligonucleotides diluted in OptiMEM. The final oligonucleotide concentration ranged from 1 nM to 25 nM. After 4 h of treatment, cells were washed in OptiMEM and 50 μl DMEM containing serum was added. Following treatment with the oligomeric compound, cells were allowed to recover for the period indicated before they were removed from the CO₂ incubator and equilibrated to room temperature for 15 min. An equal volume of highly sensitive Caspase 3/7-Glo™ Reagent (Promega) was added directly to the cells in 96 wells, and plates were incubated for 60 min before recording luminescence (luciferase activity) in Luminoskan Ascent instrument from Thermo Labsystems after further 1 min lag period. The luciferase activity is measured as Relative Light Units per seconds (RLU/s). The data were processed in the Ascent software 2.6 and graphs were drawn in excel. (See FIGS. 2 and 3).

### Example 10

### In Vitro Analysis: Antisense Inhibition of Human Mcl-1 Expression by Oligonucleotide Compounds Using Natural Uptake

Some preferred oligos (SEQ ID NOs: 50, 64, 90, 91, 92, 118, 119, 120, 121, 122, 123, 124, 125, 126, and 127) presented in Table 5 were selected and evaluated for their potential to down-regulate Mcl-1 mRNAs in 15PC3 cells using natural uptake.

15PC3 cells were seeded to a density of 120000 cells per well in 6-well cell culture plates for harvest at day 1 and day 3 and 15PC3 cells were seeded to a density of 40000 cells per well in 6-well cell culture plates for harvest at day 6. Cells were incubated at 37° C. for 24 hours whereafter oligo suspended in sterile water was added to the wells in a final oligo concentrations of 5 μM. Cells were incubated at 37° C. in the oligo containing normal growth serum for 1, 3 or 6 days before they were harvested for RNA analysis.

TABLE 5
Antisense Inhibition of Human Mcl-1 expression by oligonucleotides.

| | | Percent inhibition of |
|---|---|---|
| Test | | Mc1-1L in 15PC3 at 5μM, |
| substance | Sequence (5′-3′) | day 3 after incubation |
| SEQ ID NO: 50 | AsAsAscₛcₛaₛaₛgₛcₛcₛaₛaₛaₛGsTsA | 49 |
| SEQ ID NO: 64 | GsTsAsaₛgₛaₛcₛaₛaₛaₛcₛAsGsA | 67 |
| SEQ ID NO: 89 | GsCsTstₛcₛtₛtₛtₛcₛaₛgₛaₛcₛAsGsT | ND |
| SEQ ID NO: 90 | TsTsTscₛaₛgₛaₛcₛaₛgₛtₛgₛaₛCsTsC | 68 |
| SEQ ID NO: 91 | TsTsCsaₛgₛaₛcₛaₛgₛtₛgₛaₛcₛTsCsT | 80 |
| SEQ ID NO: 92 | TsTsAscₛaₛtₛtₛcₛtₛtₛaₛgₛtₛCsAsT | 65 |
| SEQ ID NO: 93 | AsAsAsgₛtₛcₛcₛtₛgₛaₛaₛcₛaₛCsTsT | ND |
| SEQ ID NO: 94 | CsAsGscₛtₛcₛcₛtₛaₛcₛtₛcₛcₛAsGsC | ND |
| SEQ ID NO: 118 | TsCsAsgₛaₛcₛaₛgₛtₛgₛaₛCsTsC | 88 |
| SEQ ID NO: 119 | TsCsaₛgₛaₛcₛaₛgₛtₛgₛaₛCsTsC | 85 |
| SEQ ID NO: 120 | TsTsTscₛaₛgₛaₛcₛaₛgₛtₛGsAsC | 66 |
| SEQ ID NO: 121 | TsTstₛcₛaₛgₛaₛcₛaₛgₛtₛGsAsC | 60 |
| SEQ ID NO: 122 | TsTsCsaₛgₛaₛcₛaₛgₛtₛgₛAsCsT | 69 |
| SEQ ID NO: 123 | TsTscₛaₛgₛaₛcₛaₛgₛtₛgₛAsCsT | 58 |
| SEQ ID NO: 124 | CsAsGsaₛcₛaₛgₛtₛgₛaₛcₛTsCsT | 35 |
| SEQ ID NO: 125 | CsAsgₛaₛcₛaₛgₛtₛgₛaₛcₛTsCsT | 81 |
| SEQ ID NO: 126 | AsCsAstₛtₛcₛtₛtₛaₛgₛtₛCsAsT | 85 |
| SEQ ID NO: 127 | AsCsaₛtₛtₛcₛtₛtₛaₛgₛtₛCsAsT | 67 |

As shown in Table 5, oligonucleotides of SEQ ID NOs: 64, 90, 91, 92, 118, 119, 120, 122, 125, 126, and 127 demonstrated about 65% or greater inhibition of Mcl-1 expression in these experiments and are therefore preferred. Also preferred are oligonucleotides based on the illustrated antisense oligo sequences, for example varying the length (shorter or longer) and/or nucleobase content (e.g. the type and/or proportion of analogue units), which also provide good inhibition of Mcl-1 expression.

### Example 11

### In Vitro Analysis: Caspase 3/7 Activity Induced by Antisense Inhibition of Human MCL 1 in Combination with the Histone Deacetylase Inhibitor Trichostatin A or the Alkylating Agent Cisplatin in K562 Cells

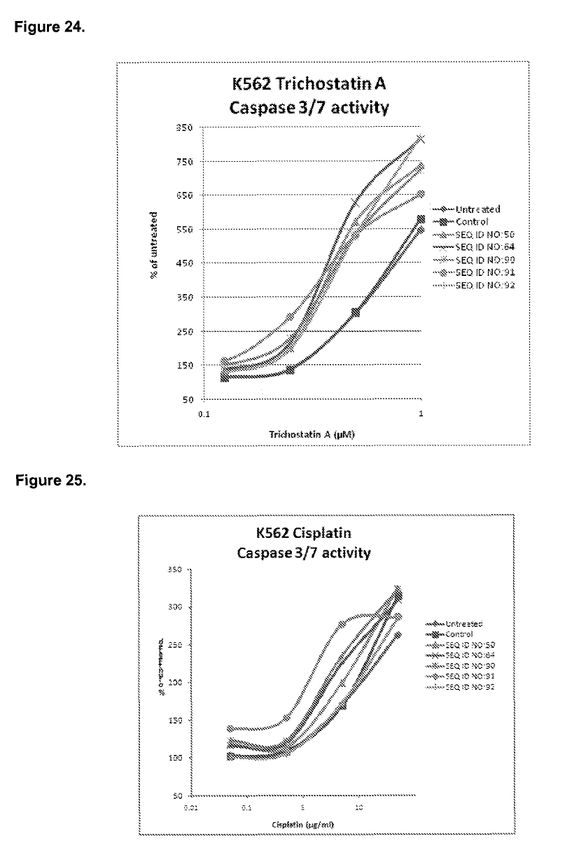

K562 chronic myeloid leukemia cells were plated at a density of 250000 cells per well in a E-well plate in 4 ml media with the final oligonucleotide concentrations of 5 μM. K562 was cultured in RPMI 1640 Medium (Sigma)+10% fetal bovine serum (FBS)+2 mM Glutamax I+gentamicin (25 μg/ml). After two days, Trichostatin A was added for final concentrations of 1 μM, 0.5 μM, 0.25 μM, and 0.125 μM, whereas Cisplatin was added for final concentrations of 50 μg/ml, 5 μg/ml, 0.5 μg/ml and 0.05 μg/ml. One day after addition of Cisplatin and Trichostatin A, 100 μl from each well in the 6-well plates was transferred to a white 96 well plate (Nunc) in 100 μl media and Caspase 3/7 activity was measured by adding 100 μl Caspase-Glo 3/7 assay (promega) using a luminometer. (See FIG. 24 and FIG. 25). As shown in FIG. 24 and FIG. 25, oligonucleotides of SEQ ID NOs: 50, 64, 90, 91, and 92 sensitizes K562 cells to apoptosis (caspase 3/7 activation) when treated with Trichostatin A and SEQ ID NOs: 50, 64, 90, and 91 sensitizes K562 cells to apoptosis (caspase 3/7 activation) when treated with Cisplatin and are therefore preferred.

### Example 12

### In Vitro Analysis: Measurement of Viable Namalwa Burkitt's Lymphoma Cells (MTS Assay) Affected by Antisense Inhibition of Human Mcl1 in Combination with the Glucocorticoid Dexamethasone

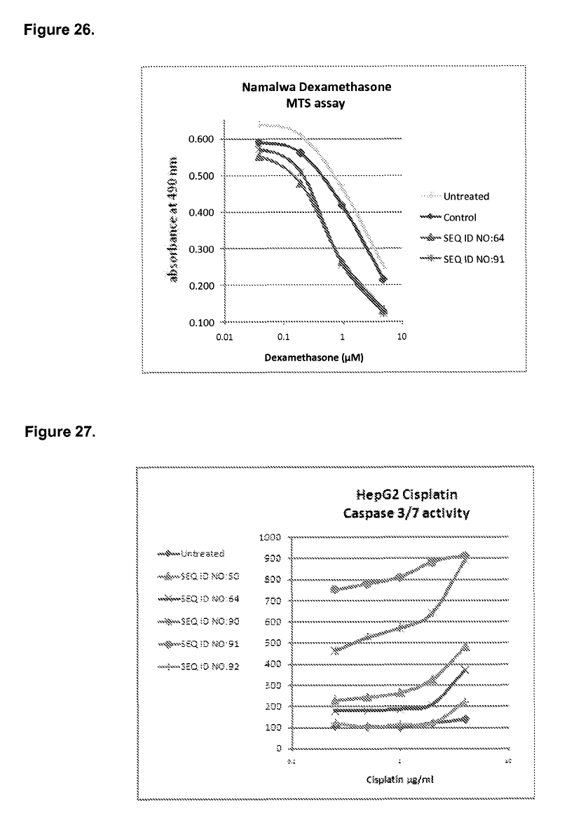

Namalwa Burkitt's lymphoma cells were plated at a density of 250000 cells per well in a E-well plate in 4 ml media with the final oligonucleotide concentrations of 5 μM. Namalwa cells were cultured in RPMI 1640 Medium (Sigma)+10% fetal bovine serum (FBS)+10 mM HEPES+4 mM Glutamax I+gentamicin (25 μg/ml). After two days, Dexamethasone was added for final concentrations of 5 μM, 1 μM, 0.2 μM, and 0.04 μM, whereas Cisplatin was added for final concentrations of 50 μg/ml, 5 μg/ml, 0.5 μg/ml and 0.05 μg/ml. Two days after addition of dexamethasone, 100 μl from each well in the 6-well plates in clear 96 well plate (Scientific Orange no. 1472030100) and viable cells were measured by adding 10 μl the tetrazolium compound [3-(4,5-dimethyl-2-yl)-5-(3-carboxymethoxyphenyl)-2-(4-sulfophenyl)-2H-tetrazolium, inner salt; MTS] and an electron coupling reagent (phenazine ethosulfate; PES) (CellTiter 96® AQueous One Solution Cell Proliferation Assay, Promega). Viable cells were measured at 490 nm in a Powerwave (Biotek Instruments) (See FIG. 26). As shown in FIG. 26, oligonucleotides of SEQ ID NOs: 64 and 91 sensitise Namalwa cells (decreased cell viability) to treatment with Dexamethasone and are therefore preferred.

### Example 13

### In Vitro Analysis: Caspase 3/7 Activity Induced by Antisense Inhibition of Human MCL1 in Combination with the Alkylating Agent Cisplatin in HepG2 Cells

HepG2 hepatocellular carcinoma cells were plated at a density of 5000 cells per well in a white 96 well plate (Nunc) in 100 μl media with the final oligonucleotide concentrations of 5 μM. HepG2 cells were cultured in EMEM (Sigma)+10% fetal bovine serum (FBS)+2 mM Glutamax I+1xNEAA+ gentamicin (25 μg/ml). After two days, Cisplatin was added for final concentrations of 4 μg/ml, 2 μg/ml, 1 μg/ml, 0.5 μg/ml and 0.25 μg/ml. Two days after addition of Cisplatin, Caspase 3/7 activity was measured by adding 100 μl Caspase-Glo 3/7 assay (promega) using a luminometer (See FIG. 27). As shown in FIG. 27, oligonucleotides of SEQ ID NOs:90 and 91 show single agent effect on Caspase 3/7 activity in HepG2 cells and SEQ ID NOs:50, 64, 90, 91 and 92 show increased effects when combined with Cisplatin and are therefore preferred.

### Example 14

### In Vitro Model: Caspase 3/7 Activation by Oligonucleotides Down-Regulating Mcl-1

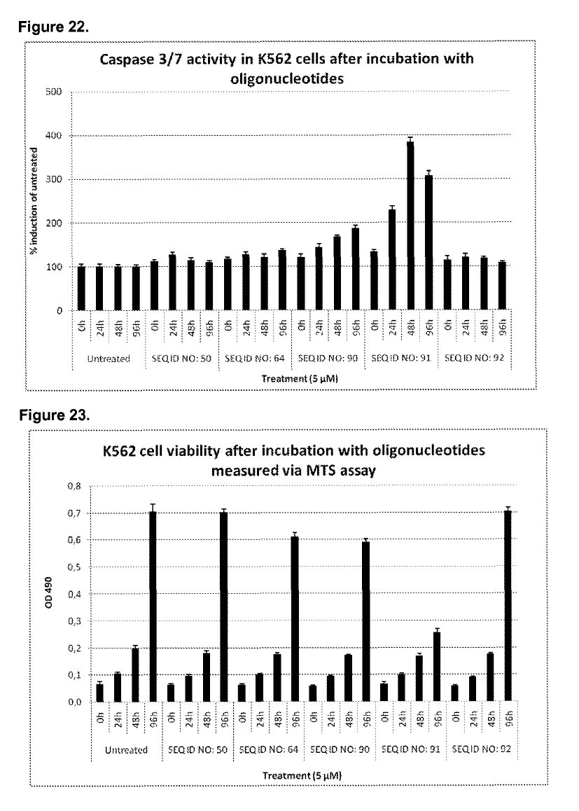

The suspension cell lines K562 and Namalwa listed in example 4 were seeded in 6-well cell culture plates at low confluence and incubated with oligo as described in example 5 such that every sample was present as a triplicate. At 0 hours, 24 hours, 48 hours, and 96 hours three 100 μl samples were drawn from every well and transferred to white-walled multiwell cell culture luminometer plates. After cooling of the plates to room temperature 100 μl Caspase 3/7-Glo™ Reagent (Promega) per well was added per well to the luminometer plates and the plates were incubated at room temperature for 60 minutes before recording luminescence (luciferase activity) in Luminoskan Ascent instrument from Thermo Labsystems after further 1 min lag period. The luciferase activity is measured as Relative Light Units per seconds (RLU/s). The data were processed in the Ascent software 2.6 and graphs were drawn in excel (see FIGS. 19 and 22). As shown in FIG. 19, oligonucleotides of SEQ ID NOs: 50, 64, 90, 91 and 92 induces Caspase 3/7 activity in the Namalwa cells. As shown in FIG. 22, oligonucleotides of SEQ ID NOs: 90 and 91 induces Caspase 3/7 activity in the K562 cells. Oligonucleotides of SEQ ID NOs: 50, 64, 90, 91 and 92 are therefore preferred.

### Example 15

### In Vitro Model: Viability Measurement Using MTS Assay

The suspension cell lines K562 and Namalwa listed in example 4 were seeded in 6-well cell culture plates at low confluence and incubated with oligo as described in example 5 such that every sample was present as a triplicate. At 0 hours, 24 hours, 48 hours, 72 hours, and 96 hours three 100 μl samples were drawn from every well and transferred to clear 96-well cell culture plates and 10 μl CellTiter 96® AQueous One Solution Reagent (CellTiter 96® AQueous One Solution Cell Proliferation, G3582, Promega) was added per well to the 96-well cell culture plates. The plates were incubated at room temperature for 3 hours before the absorbance was measured at 490 nm and 650 nm using a spectrophometer (Powerwave X, Bio-Tek Instruments).

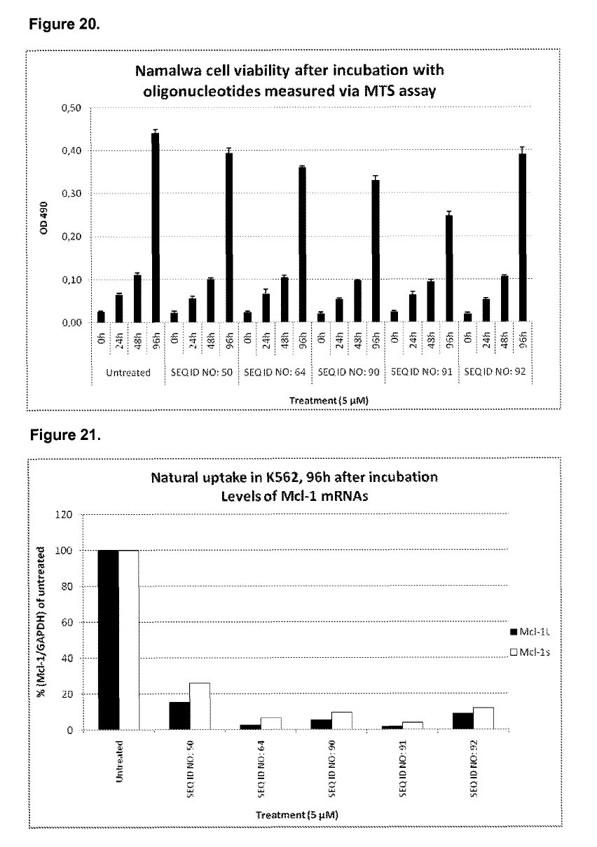

The data were processed and graphs were drawn in excel (see FIGS. 20 and 23). As shown in FIGS. 20 and 23, oligonucleotides of SEQ ID NOs: 64, 90, and 91 reduces cell viability in the Namalwa cells and the K562 cells and are therefore preferred.

### Example 16

### In Vivo Analysis: Antisense Inhibition of Mouse Mcl-1 Expression by Oligonucleotide Compounds

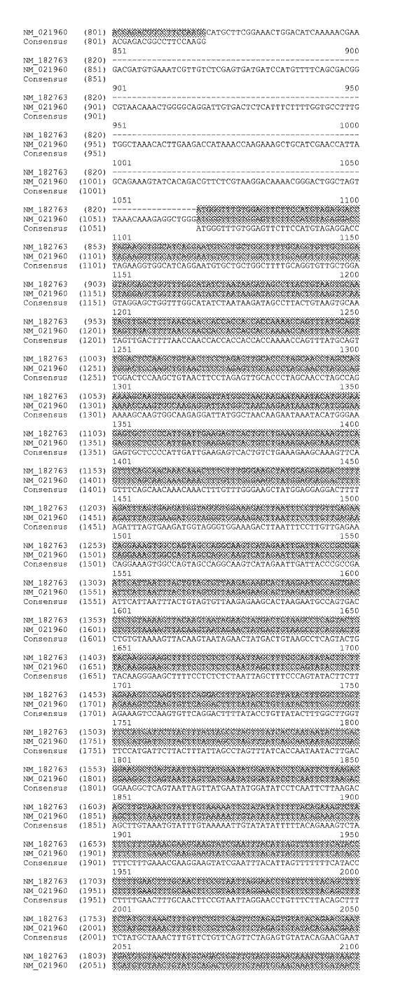

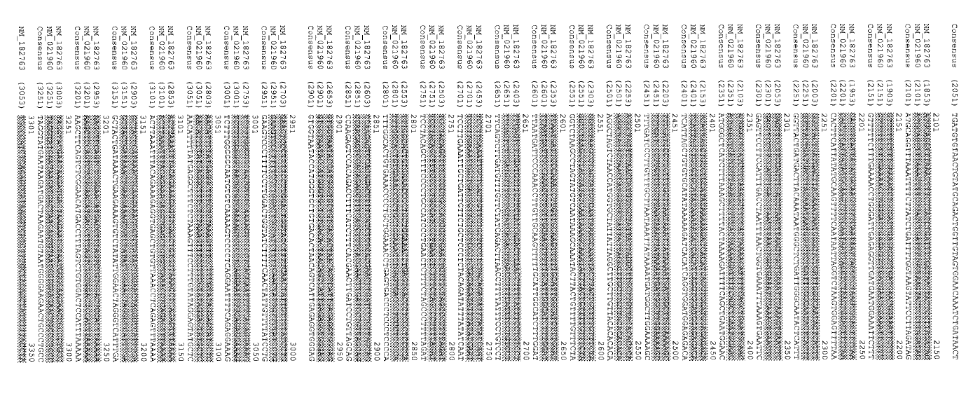

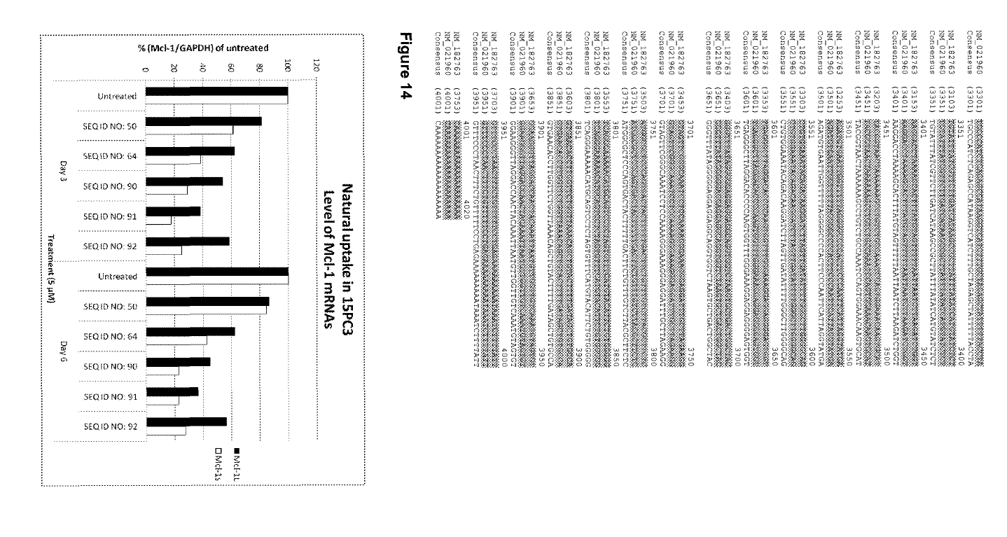

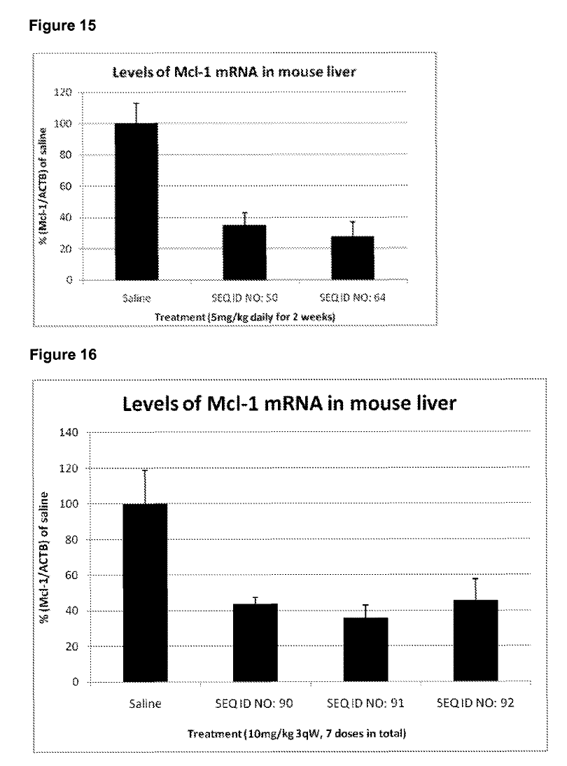

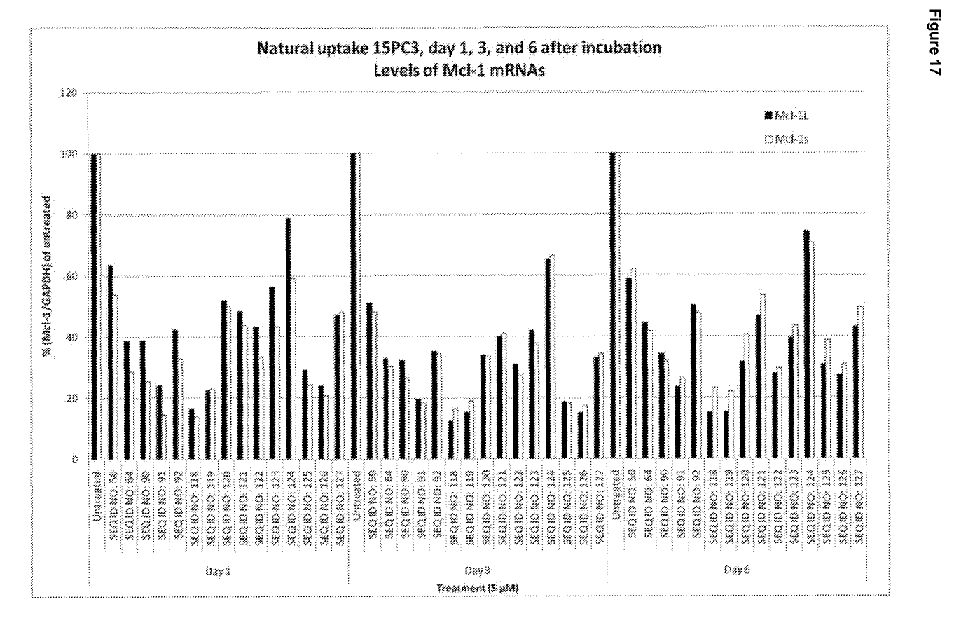

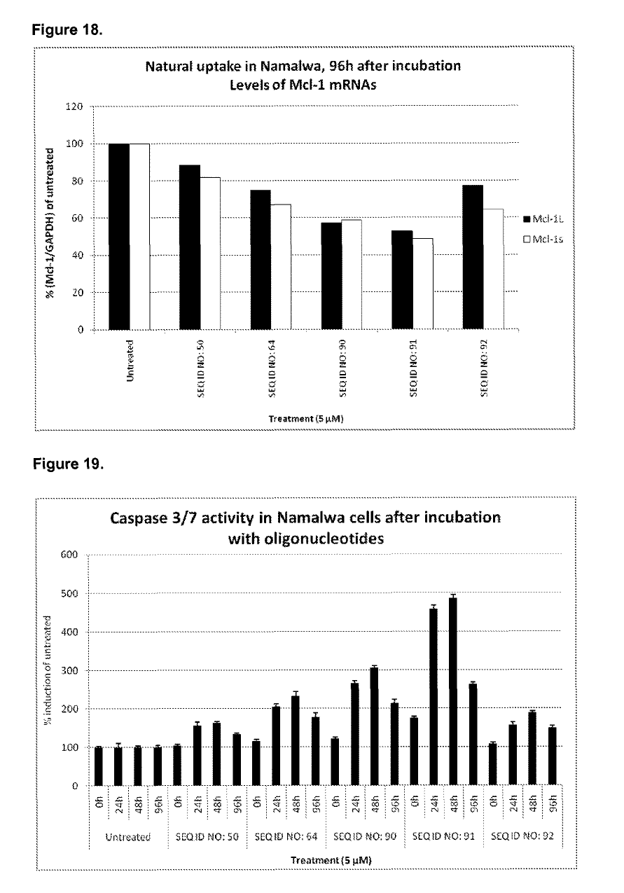

Oligonucleotides were dissolved in sterile NaCl 0.9% at 0.5 mg/ml or 1 mg/ml. Each animal was weighed and identified by ear puncture with a unique ID number before study start. The group size was 5 mice for all studies. The animals were dosed with 10 ml per kg body weight i.v. of the compound formulated in the vehicle or vehicle alone. Animals were with 5 mg/kg of oligonucleotide daily for two weeks or 10 mg/kg of oligonucleotide three times weekly for a total of 7 doses. Animals were sacrificed 24 or 48 hours after last dose and the large liver lobe was minced and submerged in RNAlater (Sigma #R-0901) for later RNA isolation, first strand synthesis and real-time PCR analysis (see FIGS. 15 and 16). As shown in FIGS. 15 and 16 oligonucleotides of SEQ ID NOs: 50, 64, 90, 91 and 92 shows pronounced down-regulation of the murine Mcl-1 mRNA in the liver tissue and are therefore preferred.

### Example 17

### Preparation of Conjugates of Oligomers with Polyethylene Glycol

The oligomers having sequences shown as SEQ ID NO: 90, 91 and 64 are functionalized on the 5′ terminus by attaching an aminoalkyl group, such as hexan-1-amine blocked with a blocking group such as Fmoc to the 5′ phosphate groups of the oligomers using routine phosphoramidite chemistry, oxidizing the resultant compounds, deprotecting them and purifying them to achieve the functionalized oligomers, respectively, having the formulas (IA)-(IC):

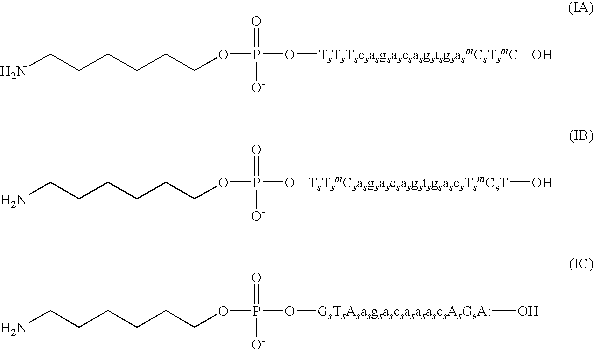

wherein the bold uppercase letters represent nucleoside analogue monomers, lowercase letters represent DNA monomers, the subscript “s” represents a phosphorothioate linkage, and ^{Me}C represents 5-methylcytosine.

A solution of activated PEG, such as the one shown in formula (II):

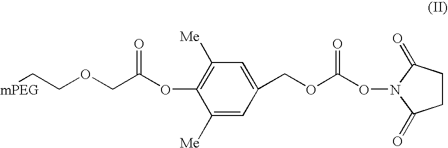

wherein the PEG moiety has an average molecular weight of 12,000, and each of the compounds of formulas (IA) and (IB) in PBS buffer are stirred in separate vessels at room temperature for 12 hours. The reaction solutions are extracted three times with methylene chloride and the combined organic layers are dried over magnesium sulphate and filtered and the solvent is evaporated under reduced pressure. The resulting residues are dissolved in double distilled water and loaded onto an anion exchange column. Unreacted PEG linker is eluted with water and the products are eluted with NH₄HCO₃ solution. Fractions containing pure products are pooled and lypophilized to yield the conjugates SEQ ID NOs: 90, 91 and 64, respectively as show in formulas (IIIA) and (IIIB):

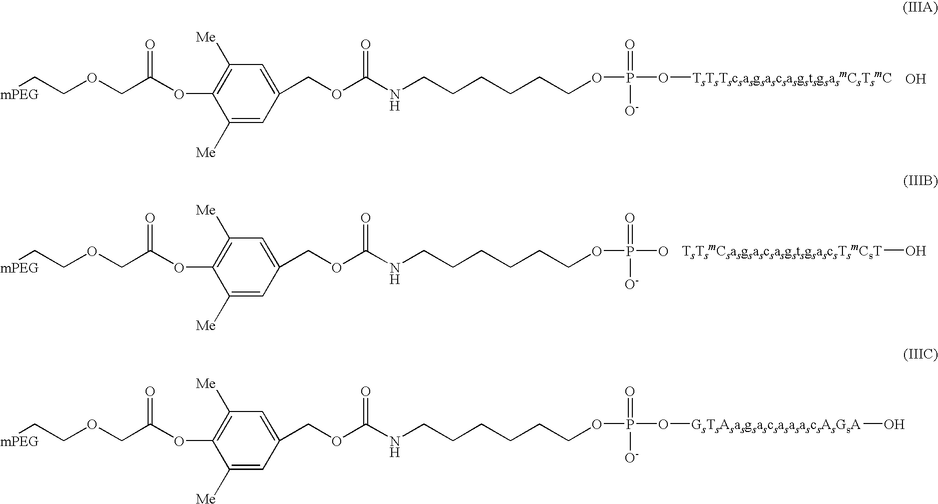

wherein each of the oligomers of SEQ ID NOs: 90, 91 and 64 is attached to a PEG polymer having average molecular weight of 12,000 via a releasable linker.

Chemical structures of PEG polymer conjugates that can be made with oligomers having sequences shown in SEQ ID NOs: 90, 91 and 64 using the process described above are respectively shown in formulas (IVA), (IVB) and (IVC):

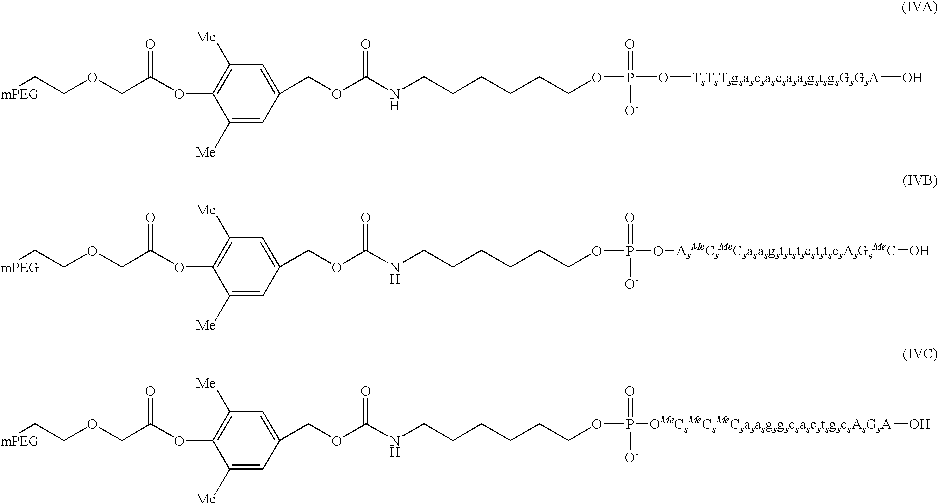

wherein bold uppercase letters represent beta-D-oxy-LNA monomers, lowercase letters represent DNA monomers, the subscript “s” represents a phosphorothioate linkage and ^{Me}C represent 5-methylcytosine.

Activated oligomers that can be used in this process to respectively make the conjugates shown in formulas (IVA), (IVB) and (IVC) have the chemical structures shown in formulas (VA), (VB) and (VC):

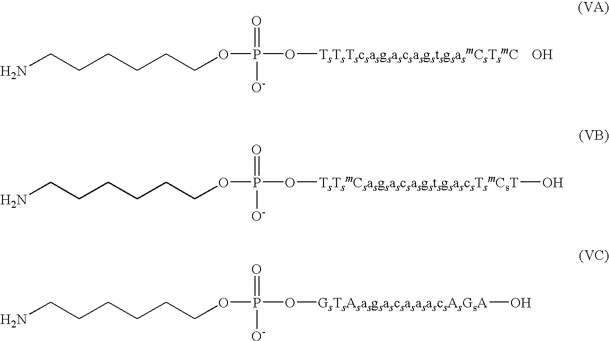

### Example 18

### In Vitro Model: Combination of Antisense Oligonucleotides Targeting Bcl-2 and Antisense Oligonucleotides Targeting Mcl-1 on Caspase 3/7 Induction and Cell Viability

Two preferred Mcl-1 targeting oligos SEQ ID NOs: 64 and 91 were combined with Bcl-2 targeting oligo SEQ ID NO 128 in 15PC3 cells using natural uptake for evaluation of caspase 3/7 induction and cell viability. The cells were treated with a final concentration of 5 μM of each oligo giving a total final concentration of 10 μM in each well. The combinations were compared to cells incubated with SEQ ID NO 64 plus scrambled control oligo, SEQ ID NO 91 plus scrambled control oligo, and SEQ ID NO 128 plus scrambled control oligo again with a final concentration of 5 μM of each oligo.

SEQ ID NO: 128 = GsAsTstₛcₛtₛgₛgₛtₛgₛtₛtₛtₛCsCsmC

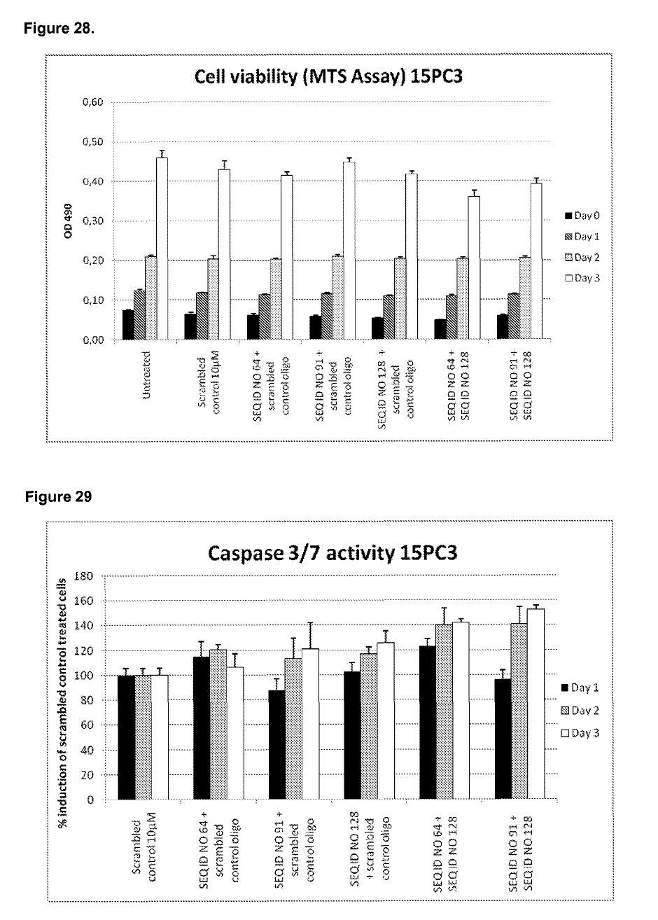

15PC3 cells were seeded at day 0 to a density of 5000 cells per well in 100 μl of normal growth medium containing the oligonucleotide combinations without any transfection reagent in clear 96-well cell culture plates for cell viability measurement and in white luminometer 96-well cell culture plates for caspase 3/7 induction measurements. The cells were incubated at 37° C. for 0, 1, 2 and 3 days. At day 0, 1, 2, and 3 cell viability was measured by adding 10 μl CellTiter 96® AQueous One Solution Reagent (CellTiter 96® AQueous One Solution Cell Proliferation, G3582, Promega) per well to the clear 96-well cell culture plates. The plates were incubated at room temperature for 1 hours before the absorbance was measured at 490 nm and 650 nm using a spectrophometer (Powerwave X, Bio-Tek Instruments) (see FIG. 28). When using a standard t-test on the results on day 3 the combination of SEQ ID NO 64+SEQ ID NO 128 showed significantly (p<0.05) less cell viability than SEQ ID NO 64+scrambled control and significantly (p<0.05) less cell viability than SEQ ID NO 128+scrambled control giving an advantage of combining the Mcl-1 targeting oligo SEQ ID NO 64 with the bcl-2 targeting oligo. The combination of SEQ ID NO 91+SEQ ID NO 128 showed significantly (p<0.05) less cell viability than SEQ ID NO 91+scrambled control and significantly (p<0.05) less cell viability than SEQ ID NO 128+scrambled control giving an advantage of combining the Mcl-1 targeting oligo SEQ ID NO 91 with the bcl-2 targeting oligo. At day 1, 2, and 3 caspase 3/7 induction was measured by cooling of the plates to room temperature and then adding 100 μl Caspase 3/7-Glo™ Reagent (Promega) per well to the luminometer plates and the plates were incubated at room temperature for 60 minutes before recording luminescence (luciferase activity) in Luminoskan Ascent instrument from Thermo Labsystems after further 1 min lag period. The luciferase activity is measured as Relative Light Units per seconds (RLU/s). The data were processed in the Ascent software 2.6 and graphs were drawn in excel (see FIG. 29). For the caspase 3/7 activity the induction was compared to the scrambled control treated cells. When using a standard t-test on the results on day 3 the combination of SEQ ID NO 64+SEQ ID NO 128 showed significantly (p<0.05) higher caspase 3/7 induction than SEQ ID NO 64+scrambled control and significantly (p<0.05) higher caspase 3/7 induction than SEQ ID NO 128+scrambled control giving an advantage of combining the Mcl-1 targeting oligo SEQ ID NO 64 with the bcl-2 targeting oligo. The combination of SEQ ID NO 91+SEQ ID NO 128 showed higher caspase 3/7 induction than SEQ ID NO 91+scrambled control and significantly (p<0.05) higher caspase 3/7 induction than SEQ ID NO 128+scrambled control giving an advantage of combining the Mcl-1 targeting oligo SEQ ID NO 91 with the bcl-2 targeting oligo.

1-19. (canceled)

20. A gapmer oligomer capable of the down-regulating expression of a mammalian Mcl-1 gene of between about 10-24 nucleotides in length, said oligomer comprising a contiguous nucleotide sequence of a total of between 10-24 nucleotides, wherein said contiguous nucleotide sequence is at least 90% homologous to a corresponding region of the reverse complement of a mammalian Mcl-1 gene; comprises at least one LNA unit; and wherein the contiguous nucleotide sequence is, over the length of the contiguous nucleotide sequence, identical to SEQ ID NOs 112, 113 or 114, or sub-sequence thereof.

21. The oligomer of claim 20, wherein the nucleotide sequence of the oligomer consists of the contiguous nucleotide sequence.

22. The oligomer of claim 20, wherein the contiguous nucleotide sequence is between about 10-18 nucleotides in length.

23. The oligomer of claim 20, wherein the contiguous nucleotide sequence is, over the length of the contiguous nucleotide sequence, identical to SEQ ID NO 77, 78 or 79, or sub-sequence thereof.

24. The oligomer of claim 20, wherein the contiguous nucleotide sequence is, over the length of the contiguous nucleotide sequence, identical to SEQ ID NO 77, 78 or 79.

25. The oligomer of claim 20, wherein the oligomer comprises a contiguous nucleotide sequence selected from the group consisting of:

| | |
|---|---|
| TsTsTscₛaₛgₛaₛcₛaₛgₛtₛgₛaₛCsTsC; | (SEQ ID NO 84) |
| GsTsAsaₛgₛaₛcₛaₛaₛaₛcₛAsGsA; | (SEQ ID NO 33) |
| and | |
| TsTsCₛaₛgₛaₛcₛaₛgₛtₛgₛaₛcₛTsCsT; | (SEQ ID NO 85) |

wherein upper case letters indicate nucleotide analogue units, subscript “s” represents phosphorothioate linkage, and lower case letters represent nucleotide units.

26. The oligomer of claim 20, wherein the oligomer comprises a contiguous nucleotide sequence selected from the group consisting of:
(SEQ ID NO 90)
TsTsTscₛaₛgₛaₛcₛaₛgₛtₛgₛa_{sMe}CsTs^{Me}C;
(SEQ ID NO 64)
GsTsAsaₛgₛaₛcₛaₛaₛaₛcₛAsGsA;
and
(SEQ ID NO 91)
TsTs^{Me}CsaₛgₛaₛcₛaₛgₛtₛgₛaₛcₛTs^{Me}CsT;
wherein uppercase letters denote LNA nucleotides, lowercase letters denote DNA monomers, the subscript “s” denotes a phosphorothioate linkage, and ^{Me}C denotes a LNA cytosine nucleotide.

27. The oligomer of claim 26, wherein the oligomer is T_{S}T_{S}T_{S}c_{S}a_{S}g_{S}a_{S}c_{S}a_{S}g_{S}t_{S}g_{S}a_{S}^{Me}C_{S}T_{S}^{Me}C (SEQ ID NO 90).

28. The oligomer of claim 26, wherein the oligomer is G_{S}T_{S}A_{S}a_{S}g_{S}a_{S}c_{S}a_{S}a_{S}a_{S}c_{S}A_{S}G_{S}A (SEQ ID NO 64).

29. The oligomer of claim 26, wherein the oligomer is T_{S}T_{S}^{Me}C_{S}a_{S}g_{S}a_{S}c_{S}a_{S}g_{S}t_{S}g_{S}a_{S}c_{S}T_{S}^{Me}C_{S}T (SEQ ID NO 91).

30. A conjugate comprising the oligomer of claim 20, and at least one non-nucleotide or non-polynucleotide moiety covalently attached to said oligomer.

31. A pharmaceutical composition comprising the oligomer of claim 20 and a pharmaceutically acceptable diluent, carrier, salt or adjuvant.

32. A method of treating a hyperproliferative disorder or inflammatory disease comprising administering a therapeutically effective amount of the oligomer of claim 20 to a subject in need thereof.

33. A method for the inhibition of Mcl-1 in a cell which is expressing Mcl-1, said method comprising administering the oligomer of claim 20 to said cell in an amount so as to inhibit Mcl-1 in said cell.

34. The oligomer of claim 20, wherein the mammalian Mcl1 gene is Mcl-1 mRNA.

35. The oligomer of claim 25, wherein said nucleotide analog units are LNA units.

36. The oligomer of claim 26, wherein said LNA nucleotides are beta-D-oxy-LNA nucleotides.

37. The oligomer of claim 26, wherein said LNA cytosine nucleotide is LNA 5-methylcytosine nucleotide.

38. A pharmaceutical composition comprising the conjugate of claim 30 and a pharmaceutically acceptable diluent, carrier, salt or adjuvant.

39. The method of claim 32, wherein said hyperproliferative disorder or inflammatory disease is selected from the group consisting of cancer, mastocytosis, and rheumatoid arthritis.

40. A method of treating a hyperproliferative disorder or inflammatory disease comprising administering a therapeutically effective amount of the conjugate of claim 30 to a subject in need thereof.

41. The method of claim 40, wherein said hyperproliferative disorder or inflammatory disease is selected from the group consisting of cancer, mastocytosis, and rheumatoid arthritis.

42. A method for the inhibition of Mcl-1 in a cell which is expressing Mcl-1, said method comprising administering the conjugate of claim 30 to said cell in an amount so as to inhibit Mcl-1 in said cell.

43. The method of claim 33, wherein said method is performed in vitro.

44. The method of claim 42, wherein said method is performed in vitro.


SEQUENCE LISTING (128 sequences)

SEQ ID NO: 1 - DNA, 3772 bp, homo sapiens
caccccgtaggactggccgccctaaaaccgtgataaaggagctgctcgccacttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaaaaaaaaaaaaaaaaaa

SEQ ID NO: 2 - DNA, 16 bp, artificial
  Oligomer sequence motif/LNA Oligomer
aagccagcagcacatt

SEQ ID NO: 3 - DNA, 16 bp, artificial
  Oligomer sequence motif/LNA Oligomer
ctactccagcaacacc

SEQ ID NO: 4 - DNA, 16 bp, artificial
  Oligomer sequence motif/LNA Oligomer
aaggctatcttattag

SEQ ID NO: 5 - DNA, 16 bp, artificial
  Oligomer sequence motif/LNA Oligomer
ctatcttattagatat

SEQ ID NO: 6 - DNA, 16 bp, artificial
  Oligomer sequence motif/LNA Oligomer
tcttattagatatgcc

SEQ ID NO: 7 - DNA, 16 bp, artificial
  Oligomer sequence motif/LNA Oligomer
ttcccatgtatttatt

SEQ ID NO: 8 - DNA, 16 bp, artificial
  Oligomer sequence motif/LNA Oligomer
atgtatttattcttgt

SEQ ID NO: 9 - DNA, 16 bp, artificial
  Oligomer sequence motif/LNA Oligomer
tttattcttgttagcc

SEQ ID NO: 10 - DNA, 16 bp, artificial
  Oligomer sequence motif/LNA Oligomer
aaaccaagccaaagta

SEQ ID NO: 11 - DNA, 14 bp, artificial
  Oligomer sequence motif/LNA Oligomer
actaggctaataaa

SEQ ID NO: 12 - DNA, 16 bp, artificial
  Oligomer sequence motif/LNA Oligomer
aaatagactttctgta

SEQ ID NO: 13 - DNA, 15 bp, artificial
  Oligomer sequence motif/LNA Oligomer
aatagcaccatggtt

SEQ ID NO: 14 - DNA, 16 bp, artificial
  Oligomer sequence motif/LNA Oligomer
tttcaaatgaccctag

SEQ ID NO: 15 - DNA, 16 bp, artificial
  Oligomer sequence motif/LNA Oligomer
aactacataaagtgct

SEQ ID NO: 16 - DNA, 14 bp, artificial
  Oligomer sequence motif/LNA Oligomer
gtaagacaaacaga

SEQ ID NO: 17 - DNA, 15 bp, artificial
  Oligomer sequence motif/LNA Oligomer
gagataatctccagc

SEQ ID NO: 18 - DNA, 14 bp, artificial
  Oligomer sequence motif/LNA Oligomer
gactccttactgga

SEQ ID NO: 19 - DNA, 16 bp, artificial
  Oligomer sequence motif/LNA Oligomer
aagccagcagcacatt

SEQ ID NO: 20 - DNA, 16 bp, artificial
  Oligomer sequence motif/LNA Oligomer
ctactccagcaacacc

SEQ ID NO: 21 - DNA, 16 bp, artificial
  Oligomer sequence motif/LNA Oligomer
aaggctatcttattag

SEQ ID NO: 22 - DNA, 16 bp, artificial
  Oligomer sequence motif/LNA Oligomer
ctatcttattagatat

SEQ ID NO: 23 - DNA, 16 bp, artificial
  Oligomer sequence motif/LNA Oligomer
tcttattagatatgcc

SEQ ID NO: 24 - DNA, 16 bp, artificial
  Oligomer sequence motif/LNA Oligomer
ttcccatgtatttatt

SEQ ID NO: 25 - DNA, 16 bp, artificial
  Oligomer sequence motif/LNA Oligomer
atgtatttattcttgt

SEQ ID NO: 26 - DNA, 16 bp, artificial
  Oligomer sequence motif/LNA Oligomer
tttattcttgttagcc

SEQ ID NO: 27 - DNA, 16 bp, artificial
  Oligomer sequence motif/LNA Oligomer
aaaccaagccaaagta

SEQ ID NO: 28 - DNA, 14 bp, artificial
  Oligomer sequence motif/LNA Oligomer
actaggctaataaa

SEQ ID NO: 29 - DNA, 16 bp, artificial
  Oligomer sequence motif/LNA Oligomer
aaatagactttctgta

SEQ ID NO: 30 - DNA, 15 bp, artificial
  Oligomer sequence motif/LNA Oligomer
aatagcaccatggtt

SEQ ID NO: 31 - DNA, 16 bp, artificial
  Oligomer sequence motif/LNA Oligomer
tttcaaatgaccctag

SEQ ID NO: 32 - DNA, 16 bp, artificial
  Oligomer sequence motif/LNA Oligomer
aactacataaagtgct

SEQ ID NO: 33 - DNA, 14 bp, artificial
  Oligomer sequence motif/LNA Oligomer
gtaagacaaacaga

SEQ ID NO: 34 - DNA, 15 bp, artificial
  Oligomer sequence motif/LNA Oligomer
gagataatctccagc

SEQ ID NO: 35 - DNA, 14 bp, artificial
  Oligomer sequence motif/LNA Oligomer
gactccttactgga

SEQ ID NO: 36 - DNA, 16 bp, artificial
  Oligomer sequence motif/LNA Oligomer
aagccagcagcacatt

SEQ ID NO: 37 - DNA, 14 bp, artificial
  Oligomer sequence motif/LNA Oligomer
agccagcagcacat

SEQ ID NO: 38 - DNA, 12 bp, artificial
  Oligomer sequence motif/LNA Oligomer
gccagcagcaca

SEQ ID NO: 39 - DNA, 16 bp, artificial
  Oligomer sequence motif/LNA Oligomer
ctactccagcaacacc

SEQ ID NO: 40 - DNA, 16 bp, artificial
  Oligomer sequence motif/LNA Oligomer
aaggctatcttattag

SEQ ID NO: 41 - DNA, 16 bp, artificial
  Oligomer sequence motif/LNA Oligomer
ctatcttattagatat

SEQ ID NO: 42 - DNA, 16 bp, artificial
  Oligomer sequence motif/LNA Oligomer
tcttattagatatgcc

SEQ ID NO: 43 - DNA, 14 bp, artificial
  Oligomer sequence motif/LNA Oligomer
cttattagatatgc

SEQ ID NO: 44 - DNA, 12 bp, artificial
  Oligomer sequence motif/LNA Oligomer
ttattagatatg

SEQ ID NO: 45 - DNA, 16 bp, artificial
  Oligomer sequence motif/LNA Oligomer
ttcccatgtatttatt

SEQ ID NO: 46 - DNA, 16 bp, artificial
  Oligomer sequence motif/LNA Oligomer
atgtatttattcttgt

SEQ ID NO: 47 - DNA, 16 bp, artificial
  Oligomer sequence motif/LNA Oligomer
tttattcttgttagcc

SEQ ID NO: 48 - DNA, 14 bp, artificial
  Oligomer sequence motif/LNA Oligomer
ttattcttgttagc

SEQ ID NO: 49 - DNA, 12 bp, artificial
  Oligomer sequence motif/LNA Oligomer
tattcttgttag

SEQ ID NO: 50 - DNA, 16 bp, artificial
  Oligomer sequence motif/LNA Oligomer
aaaccaagccaaagta

SEQ ID NO: 51 - DNA, 14 bp, artificial
  Oligomer sequence motif/LNA Oligomer
aaccaagccaaagt

SEQ ID NO: 52 - DNA, 12 bp, artificial
  Oligomer sequence motif/LNA Oligomer
accaagccaaag

SEQ ID NO: 53 - DNA, 14 bp, artificial
  Oligomer sequence motif/LNA Oligomer
actaggctaataaa

SEQ ID NO: 54 - DNA, 16 bp, artificial
  Oligomer sequence motif/LNA Oligomer
aaatagactttctgta

SEQ ID NO: 55 - DNA, 15 bp, artificial
  Oligomer sequence motif/LNA Oligomer
aatagcaccatggtt

SEQ ID NO: 56 - DNA, 13 bp, artificial
  Oligomer sequence motif/LNA Oligomer
atagcaccatggt

SEQ ID NO: 57 - DNA, 11 bp, artificial
  Oligomer sequence motif/LNA Oligomer
tagcaccatgg

SEQ ID NO: 58 - DNA, 16 bp, artificial
  Oligomer sequence motif/LNA Oligomer
tttcaaatgaccctag

SEQ ID NO: 59 - DNA, 14 bp, artificial
  Oligomer sequence motif/LNA Oligomer
ttcaaatgacccta

SEQ ID NO: 60 - DNA, 12 bp, artificial
  Oligomer sequence motif/LNA Oligomer
tcaaatgaccct

SEQ ID NO: 61 - DNA, 16 bp, artificial
  Oligomer sequence motif/LNA Oligomer
aactacataaagtgct

SEQ ID NO: 62 - DNA, 14 bp, artificial
  Oligomer sequence motif/LNA Oligomer
actacataaagtgc

SEQ ID NO: 63 - DNA, 12 bp, artificial
  Oligomer sequence motif/LNA Oligomer
ctacataaagtg

SEQ ID NO: 64 - DNA, 14 bp, artificial
  Oligomer sequence motif/LNA Oligomer
gtaagacaaacaga

SEQ ID NO: 65 - DNA, 12 bp, artificial
  Oligomer sequence motif/LNA Oligomer
taagacaaacag

SEQ ID NO: 66 - DNA, 15 bp, artificial
  Oligomer sequence motif/LNA Oligomer
gagataatctccagc

SEQ ID NO: 67 - DNA, 14 bp, artificial
  Oligomer sequence motif/LNA Oligomer
gagataatctccag

SEQ ID NO: 68 - DNA, 12 bp, artificial
  Oligomer sequence motif/LNA Oligomer
gataatctccag

SEQ ID NO: 69 - DNA, 14 bp, artificial
  Oligomer sequence motif/LNA Oligomer
gactccttactgga

SEQ ID NO: 70 - DNA, 4020 bp, Homo sapiens
caccccgtaggactggccgccctaaaaccgtgataaaggagctgctcgccacttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaaaaaaaaaaaaaaaaaa

SEQ ID NO: 71 - protein, 350 aa, homo sapiens
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGIRNVLLAFAGVAGVGAGLAYLIR

SEQ ID NO: 72 - protein, 271 aa, Homo sapiens
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGWVCGVLPCRGPRRWHQECAAGFCRCCWSRSWFGISNKIALL

SEQ ID NO: 73 - DNA, 3498 bp, Mus musculus
gggaagtcctcgcctgcgtcagcacggccctaaggcggcggcagggaacggccttcctcactcctgacttccgcctgcctccggtctggagtcgcggccttccccgctccttcccctcagcctgcggcgtccgaccatgtttggcctgcggagaaacgcggtcatcggcttgaacctgtactgcggcggcgccagcctcggcgcgggcggcggttctccggcaggggcgcgcctggtggccgaggaggccaaggcgcggcgcgaggggggaggggaggccgccctgctgcccggcgcgcgggtggtcgcccggccgccgcccgtgggcgccgaggaccccgacgtcaccgcgtcggccgaaaggcggctgcataagtcgcccggcctcctcgccgtgccgcccgaggagatggccgcgtcggccgccgccgccatcgtgtctccggaggaggaactggacggctgcgagccggaggccatcggcaagcgcccggccgtgctgcccctcctggagcgcgtgagcgaggcggccaagagctccggggccgacggctctctgccctccacgccgccgccgcccgaggaggaagaggacgacctataccgccagtcgctggagatcatctcgcgctacttgcgggagcaggcgaccggctccaaggactcgaagcctctgggcgaggcgggcgcggcgggccggagagcgctggagaccctgcggcgcgtgggcgacggcgtgcagcgcaaccacgagacggccttccagggcatgctccggaaactggacattaaaaacgaaggcgatgttaaatctttttctcgagtaatggtccatgttttcaaagatggcgtaacaaactggggcaggattgtgactcttatttctttcggtgcctttgtggccaaacacttaaagagcgtaaaccaagaaagcttcatcgaaccattagcagaaactatcacagatgttcttgtaaggacgaaacgggactggcttgtcaaacaaagaggctgggatgggtttgtggagttcttccacgtacaggacctagaaggcggcatcagaaatgtgctgctggcttttgcgggtgttgctggagtaggggctggtctggcatatctaataagatagccttgtgagtgcaataggggactcttaaagctccagccaccaaactacatgcatctgtgaaaacatgtgtatttatgaaggtggacttgaagctgcccaggattttaacagtccagttctactgtagcaacatagcaaaaagaaagtggctacaggattgtggctaacaagaataaatacatgggaaaagtgctccccctggaagagtcactgtctgaatgaagcaaagttccctctcagcaaacactgagaggccatggagaaggacttctagaatgaatgaaaggggtggatggaaaggtttgatttcctatctagagatggaagaggggccagtcatcaggctagtcacaaagctcaataaatatccattactctgctcagagtgttgagagagaagccctaattaacaacgttggtgacttgtgtaaaatggatttgtaacctacaagtcaccaaacgatgactagaagctgcagtgctgtacaggaatgtgaagggaggcctctgagcagtccagggtgtgcttgacaaagtcccaagtgctcaggacttttagccctgtctactttggcttggtttggatgattcttaagtttattagcctagtgatggccaaaaagtacttgacttaaggttcactaattagttacaaaactgaacagcctcgatttttaagaaaatttatgaaatgtatttgtctgtaaaaattgtatatatttttacagaaagtctatttctttgaacataagagggaagtcttggatttagtttttttccatacccttttgaaatttgcagcttctctagttagaaatgtatttcttacagctttttaagactttgtcatccgttctagtgtatatgcagagcctgttgtgtgtgtggactggttatagatttataactatgcagggttaattatccaatcttttttttttgtatttaaagatcacaggtagatagatgtaagacatttgatcccctgttgacttacaaaattgtaaatggtaaagcaggctagagtcttaaccatggtgctattcactgggtttacttgtttaacacaagtttatacctggtgtcaataaaacaaatatgtatttcttgtctactaaggattgccaagctttgttttgaatttctgtattggtctctaggtgtagcctttacttcctttactgttggcgtgttatgctcccagttcccctatagagtcgtctacacttcctccctgtgctcttcactggccattttagttatctgcattagaactctcaccccaacccccccaaaactttgaacctaaataacccctcccccatcctaatcagagtcagcacagctttcctgtcagaggataggaacacttgctctttgagccccgtgaccgacagtgaccagggattggcattcttggattggcatgctttccattgttactttgtctattttggtaagaaaaacatcaaaggtccccttgggaatttcaaaggttggaaattatacgtttatacttttattagtttgctccaagtatgactgtgttcacttaagtttagagaaacactgggttttgctaaactcggttaacaaaagccactggtaactgaaggtatttaagctagggtcatttgaaagcttcagcctcagaatgtgacctttagtcagggctgcagataaaaataggcaggaatcaaatgactaagaatgtaatagggaagaagtgccctgcctgccagcttgggagtgatttgtacctgtgtatttatccttgatcatagtttgcttatttatgtttaactcttcggacttcagagcactttatgtagttgaataaaccctagaggttgaggtgactaaaaggttagcctctcataacccagccatggaagttttgtttactaggagcctgacttcccagctcacaaagggtgaaatgaaagctgaagtacagacaggatcttagtaggtattttaggtccagggccatgaagacttggagagcgcagtggctgggcaaggggctgctttggagggaagaagcatgtggtctgagtgctgactagatgaatagttgaggcacatccttcagagaagagggaggggctgcttggaaagatggctctcagcaaaatctgtttgtcttacacgtctctcagggaaaaaacatgcagtcttcaagcatcttccatgtacattctatctgggtgaacaccttcattctggtagagcacctaacactttaacagctgttcccgaaagggttaggaccaactgcaagttaatgtgggttgtaaatgttcccctaacttctgtttcttctgaagagaaaataaacctttttccccccaaaaaaaaaaaaa

SEQ ID NO: 74 - protein, 331 aa, Mus musculus
MFGLRRNAVIGLNLYCGGASLGAGGGSPAGARLVAEEAKARREGGGEAALLPGARVVARPPPVGAEDPDVTASAERRLHKSPGLLAVPPEEMAASAAAAIVSPEEELDGCEPEAIGKRPAVLPLLERVSEAAKSSGADGSLPSTPPPPEEEEDDLYRQSLEIISRYLREQATGSKDSKPLGEAGAAGRRALETLRRVGDGVQRNHETAFQGMLRKLDIKNEGDVKSFSRVMVHVFKDGVTNWGRIVTLISFGAFVAKHLKSVNQESFIEPLAETITDVLVRTKRDWLVKQRGWDGFVEFFHVQDLEGGIRNVLLAFAGVAGVGAGLAYLIR

SEQ ID NO: 75 - DNA, 1724 bp, Macaca mulatta
gccccgaccccgccccggcccggcagccggtaggtgccgtgcgcaaccctccggaagctgccgcccctttccccttttatgggaatacttttttaaaaaaattaaagttcgctgacgccacccagtaggactagccgccctaaaaccgtgatcaaggagctcctcgccacttctcacttccgcttccttccagtaaggagtcggggtcttccccagttttcttgccaggcggcggcggcggcggcgactggcgatgtttggcctcaaaagaaacgcggtaatcggactcaacctctactgtgggggggccggcttgggggccggcagcggcggcgccacccctccgggagggcggcttttggctacggagaaggaggcctcggcccggcgagagatagggggaggggaggccggcacggtgattggcggaagcgccggcgcaagccccccggccgccctcacgccagacgcccggagggtcgcgcggccgccgcccattggcgcggaggtccccgacgtcaccgcgagccccgcgaggctgcttttctttgcgcccacccgccgcgcgtcgccgcttgaggagatggaagccccggccgccgacgccatcatgtcgcccgaagaggagctggacgggtacgagccggagcctctcgggaagcggccggctgtcctgcccctgctggagttggtcggggaatctggtaatagccccagtacggatgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggctctggagaccttacgacgggttggggatggcgtgcagcgcaaccacgagacggccttccaaggcatgcttcggaaactggacatcaaaaacgaagacgatgtcaaatctttgtctcgagtgatggtccatgttttcagcgacggcgtaacaaactggggcaggattgtgactctcatttcttttggtgcctttgtggcgaaacacttgaagaccataaaccaagaaagctgcatcgaaccattagcagaaagtatcacagacgttctcgtaaggacaaaacgggactggctagttaaacaaagaggctgggtaagttttccttaaggatgaaaggggacttggagtggaagtagaatgaaggatttatttagagaggtggggatatctaaaggtttttatgacgcacggctgtttgcaggctctaactaaaggaccattgtttatttgataatgatttaagtagtggatccttagagatagtggtatggcggtcttgaattgtatcaaaaatcttggttttctctaggctattttttgttccagttcagttgaatactcttcagtggattcaaaccatgaagaaataagtcaccaggggaggatagctgaaatacattcctaaggcgctccctgttttaattgagaagatacggggtggaccttgcgttttaaacagaaacccagatctgatgcaggatgtacttaaccacgttgagaaaaactgatcttcgcagttgaggtgttactgaaatattaggtggtggagatttgagaataagtgttttagtcttttacttcatgggaactctggaagtcccttggtttggataaatcctaatatgac

SEQ ID NO: 76 - protein, 316 aa, Macaca mulatta
MFGLKRNAVIGLNLYCGGAGLGAGSGGATPPGGRLLATEKEASARREIGGGEAGTVIGGSAGASPPAALTPDARRVARPPPIGAEVPDVTASPARLLFFAPTRRASPLEEMEAPAADAIMSPEEELDGYEPEPLGKRPAVLPLLELVGESGNSPSTDGSLPSTPPPAEEEEDELYRQSLEIISRYLREQATGAKDTKPMGRSGATSRKALETLRRVGDGVQRNHETAFQGMLRKLDIKNEDDVKSLSRVMVHVFSDGVTNWGRIVTLISFGAFVAKHLKTINQESCIEPLAESITDVLVRTKRDWLVKQRGWVSFP

SEQ ID NO: 77 - DNA, 16 bp, artificial
  Oligomer sequence motif/LNA Oligomer
gcttctttcagacagt

SEQ ID NO: 78 - DNA, 16 bp, artificial
  Oligomer sequence motif/LNA Oligomer
tttcagacagtgactc

SEQ ID NO: 79 - DNA, 16 bp, artificial
  Oligomer sequence motif/LNA Oligomer
ttcagacagtgactct

SEQ ID NO: 80 - DNA, 16 bp, artificial
  Oligomer sequence motif/LNA Oligomer
ttacattcttagtcat

SEQ ID NO: 81 - DNA, 16 bp, artificial
  Oligomer sequence motif/LNA Oligomer
aaagtcctgaacactt

SEQ ID NO: 82 - DNA, 16 bp, artificial
  Oligomer sequence motif/LNA Oligomer
cagctcctactccagc

SEQ ID NO: 83 - DNA, 16 bp, artificial
  Oligomer sequence motif/LNA Oligomer
gcttctttcagacagt

SEQ ID NO: 84 - DNA, 16 bp, artificial
  Oligomer sequence motif/LNA Oligomer
tttcagacagtgactc

SEQ ID NO: 85 - DNA, 16 bp, artificial
  Oligomer sequence motif/LNA Oligomer
ttcagacagtgactct

SEQ ID NO: 86 - DNA, 16 bp, artificial
  Oligomer sequence motif/LNA Oligomer
ttacattcttagtcat

SEQ ID NO: 87 - DNA, 16 bp, artificial
  Oligomer sequence motif/LNA Oligomer
aaagtcctgaacactt

SEQ ID NO: 88 - DNA, 16 bp, artificial
  Oligomer sequence motif/LNA Oligomer
cagctcctactccagc

SEQ ID NO: 89 - DNA, 16 bp, artificial
  Oligomer sequence motif/LNA Oligomer
gcttctttcagacagt

SEQ ID NO: 90 - DNA, 16 bp, artificial
  Oligomer sequence motif/LNA Oligomer
tttcagacagtgactc

SEQ ID NO: 91 - DNA, 16 bp, artificial
  Oligomer sequence motif/LNA Oligomer
ttcagacagtgactct

SEQ ID NO: 92 - DNA, 16 bp, artificial
  Oligomer sequence motif/LNA Oligomer
ttacattcttagtcat

SEQ ID NO: 93 - DNA, 16 bp, artificial
  Oligomer sequence motif/LNA Oligomer
aaagtcctgaacactt

SEQ ID NO: 94 - DNA, 16 bp, artificial
  Oligomer sequence motif/LNA Oligomer
cagctcctactccagc

SEQ ID NO: 95 - DNA, 24 bp, artificial
  Oligomer sequence motif/LNA Oligomer
gcaaaagccagcagcacattcctg

SEQ ID NO: 96 - DNA, 24 bp, artificial
  Oligomer sequence motif/LNA Oligomer
gctcctactccagcaacacctgca

SEQ ID NO: 97 - DNA, 24 bp, artificial
  Oligomer sequence motif/LNA Oligomer
cagtaaggctatcttattagatat

SEQ ID NO: 98 - DNA, 24 bp, artificial
  Oligomer sequence motif/LNA Oligomer
aaggctatcttattagatatgcca

SEQ ID NO: 99 - DNA, 24 bp, artificial
  Oligomer sequence motif/LNA Oligomer
gctatcttattagatatgccaaac

SEQ ID NO: 100 - DNA, 24 bp, artificial
  Oligomer sequence motif/LNA Oligomer
actcttcccatgtatttattcttg

SEQ ID NO: 101 - DNA, 24 bp, artificial
  Oligomer sequence motif/LNA Oligomer
tcccatgtatttattcttgttagc

SEQ ID NO: 102 - DNA, 24 bp, artificial
  Oligomer sequence motif/LNA Oligomer
tgtatttattcttgttagccataa

SEQ ID NO: 103 - DNA, 24 bp, artificial
  Oligomer sequence motif/LNA Oligomer
atggaaaccaagccaaagtataac

SEQ ID NO: 104 - DNA, 24 bp, artificial
  Oligomer sequence motif/LNA Oligomer
gataaactaggctaataaagtaag

SEQ ID NO: 105 - DNA, 24 bp, artificial
  Oligomer sequence motif/LNA Oligomer
aaagaaatagactttctgtaaaaa

SEQ ID NO: 106 - DNA, 24 bp, artificial
  Oligomer sequence motif/LNA Oligomer
taataatagcaccatggttagact

SEQ ID NO: 107 - DNA, 24 bp, artificial
  Oligomer sequence motif/LNA Oligomer
aagctttcaaatgaccctagttcc

SEQ ID NO: 108 - DNA, 24 bp, artificial
  Oligomer sequence motif/LNA Oligomer
taaaaactacataaagtgctttta

SEQ ID NO: 109 - DNA, 24 bp, artificial
  Oligomer sequence motif/LNA Oligomer
gaagcgtaagacaaacagaagtca

SEQ ID NO: 110 - DNA, 24 bp, artificial
  Oligomer sequence motif/LNA Oligomer
accgagagataatctccagcgact

SEQ ID NO: 111 - DNA, 24 bp, artificial
  Oligomer sequence motif/LNA Oligomer
accccgactccttactggaaggaa

SEQ ID NO: 112 - DNA, 24 bp, artificial
  Oligomer sequence motif/LNA Oligomer
ctttgcttctttcagacagtgact

SEQ ID NO: 113 - DNA, 24 bp, artificial
  Oligomer sequence motif/LNA Oligomer
cttctttcagacagtgactcttca

SEQ ID NO: 114 - DNA, 24 bp, artificial
  Oligomer sequence motif/LNA Oligomer
ttctttcagacagtgactcttcaa

SEQ ID NO: 115 - DNA, 24 bp, artificial
  Oligomer sequence motif/LNA Oligomer
cccattacattcttagtcatctta

SEQ ID NO: 116 - DNA, 24 bp, artificial
  Oligomer sequence motif/LNA Oligomer
tataaaagtcctgaacacttggac

SEQ ID NO: 117 - DNA, 24 bp, artificial
  Oligomer sequence motif/LNA Oligomer
aaaccagctcctactccagcaaca

SEQ ID NO: 118 - DNA, 14 bp, artificial
  Oligomer sequence motif/LNA Oligomer
tcagacagtgactc

SEQ ID NO: 119 - DNA, 14 bp, artificial
  Oligomer sequence motif/LNA Oligomer
tcagacagtgactc

SEQ ID NO: 120 - DNA, 14 bp, artificial
  Oligomer sequence motif/LNA Oligomer
tttcagacagtgac

SEQ ID NO: 121 - DNA, 14 bp, artificial
  Oligomer sequence motif/LNA Oligomer
tttcagacagtgac

SEQ ID NO: 122 - DNA, 14 bp, artificial
  Oligomer sequence motif/LNA Oligomer
ttcagacagtgact

SEQ ID NO: 123 - DNA, 14 bp, artificial
  Oligomer sequence motif/LNA Oligomer
ttcagacagtgact

SEQ ID NO: 124 - DNA, 14 bp, artificial
  Oligomer sequence motif/LNA Oligomer
cagacagtgactct

SEQ ID NO: 125 - DNA, 14 bp, artificial
  Oligomer sequence motif/LNA Oligomer
cagacagtgactct

SEQ ID NO: 126 - DNA, 14 bp, artificial
  Oligomer sequence motif/LNA Oligomer
acattcttagtcat

SEQ ID NO: 127 - DNA, 14 bp, artificial
  Oligomer sequence motif/LNA Oligomer
acattcttagtcat

SEQ ID NO: 128 - DNA, 16 bp, artificial
  Oligomer sequence motif/LNA Oligomer
gattctggtgtttccc